(19) United States
(12) Patent Application Publication
(10) Pub. No.: US 2019/0285618 A1
(43) Pub. Date: Sep. 19, 2019

(54) EXOSOMES AND USES THEREOF

(71) Applicants: The Broad Institute, Inc., Cambridge, MA (US); Massachusetts Institute of Technology, Cambridge, MA (US); President and Fellows of Harvard College, Cambridge, MA (US)

(72) Inventors: Aviv Regev, Cambridge, MA (US); George Church, Cambridge, MA (US); Dmitry Ter-Ovanesyan, Cambridge, MA (US); Emma Kowal, Cambridge, MA (US)

(73) Assignees: The Broad Institute, Inc., Cambridge, MA (US); Massachusetts Institute of Technology, Cambridge, MA (US); President and Fellows of Harvard College, Cambridge, MA (US)

(21) Appl. No.: 16/345,124

(22) PCT Filed: Oct. 26, 2017
(86) PCT No.: PCT/US2017/058617

§ 371 Date: Apr. 25, 2019

Related U.S. Application Data
(60) Provisional application No. 62/413,386, filed on Oct. 26, 2016.

(51) Int. Cl. G01N 33/50; C12N 15/10; G01N 33/574

(52) U.S. Cl. CPC G01N 33/5076; G01N 33/57492; C12N 15/1013

(57) ABSTRACT

The present invention relates to the isolation and purification of exosomes from biological samples, and to methods for extracting RNA contained therein. In particular, the present invention relates to a method for the isolation of cell type-specific exosomes or cell-subtype-specific exosomes from a biological sample, and to realted applications in the filed of diagnostics.

### CROSS-REFERENCE TO RELATED APPLICATIONS

This application claims the benefit of U.S. Provisional Application No. 62/413,386 filed on Oct. 26, 2016. The entire contents of the above-identified application is hereby fully incorporated herein by reference.

### STATEMENT REGARDING FEDERALLY SPONSORED RESEARCH

This invention was made with government support under grant numbers HG006193 and HG005550 awarded by the National Institutes of Health. The government has certain rights in the invention.

### FIELD OF THE INVENTION

The present invention relates to the isolation and purification of exosomes from biological samples, and to methods for extracting RNA contained therein. In particular, the present invention relates to a method for the isolation of cell type-specific exosomes or cell-subtype-specific exosomes from a biological sample. The present invention provides methods and uses for the purification of exosomes, and applications in the filed of diagnosis, prognosis.

The present invention also relates to a method for the selection of an antibody for the isolation of cell type-specific exosomes or cell-subtype-specific exosomes from a biological sample.

### BACKGROUND OF THE INVENTION

Exosomes are small extracellular vesicles that have been shown to contain RNA.

Exosomes can be isolated using ultracentrifugation steps. However, purified exosomes have proven to be difficult to isolate. In particular, the presence of cellular debris amounts to ‘contaminant’ in a preparation, jeopardizing genetic and biochemical analysis of exosomes. While exosomes are isolated using ultracentrifugation as described herein, other methods such as filtration, chemical precipitation, size exclusion chromatography, microfluidics are known in the art.

Further, RNA content of exosomes was previously reported as uncorrelated to corresponding cellular RNA content (Skog J, Würdinger T, van Rijn S, Meijer D H, Gainche L, Sena-Esteves M, Curry W T Jr, Carter B S, Krichevsky A M, Breakefield X O. Nat Cell Biol. 2008 December; 10(12):1470-6. doi: 10.1038/ncb1800. Epub 2008 Nov. 16.).

Citation or identification of any document in this application is not an admission that such document is available as prior art to the present invention.

### SUMMARY OF THE INVENTION

It would be of interest to provide methods that allow to establish a relationship between exosomal RNA content and corresponding cellular RNA content. This would have broad diagnostic and prognostic applications.

The present invention relates to a method for the selection of an antibody for the isolation of cell type-specific exosomes or cell-subtype-specific exosomes from a biological sample. In one aspect of the invention, the invention pertains to a method for the selection of an antibody for the isolation of cell type-specific exosomes or cell-subtype-specific exosomes from a biological sample, said method comprising:
(a) providing a biological sample comprising exosomes from a cell population,
(b) selecting one or more cell type-specific or cell-subtype-specific membrane marker(s) present on the surface of the exosomes to be isolated,
(c) selecting an antibody against each of the one or more the cell type-specific or cell-subtype-specific membrane marker(s) of step (b), wherein said antibody (resp. each of said antibodies)has (have):
   a capture rate of 30% or more for the cell type-specific or cell-subtype-specific membrane marker, and
   a specificity of 70% or more for the cell type-specific or cell-subtype-specific membrane marker.

The present invention also relates to a method for the isolation of cell type-specific exosomes or cell-subtype-specific exosomes from a biological sample. In one aspect, the present invention relates to a method for the isolation of cell type-specific exosomes or cell-subtype-specific exosomes from a biological sample, said method comprising:
(a) providing a biological sample comprising exosomes from a cell population,
(b) selecting one or more cell type-specific or cell-subtype-specific membrane marker(s) present on the surface of the exosomes to be isolated,
(c) selecting an antibody against each of the one or more the cell type-specific or cell-subtype-specific membrane marker(s) of step (b), wherein said antibody (resp. each of said antibodies) has (have):
   a capture rate of 30% or more for the cell type-specific or cell-subtype-specific membrane marker, and
   a specificity of 70% or more for the cell type-specific or cell-subtype-specific membrane marker,
(d) performing immuno-isolation of exosomes from the biological sample of step (a) using the antibody or antibodies of step (c), thereby providing isolated cell type-specific exosomes or cell-subtype-specific exosomes.

In some embodiments, the antibody has a capture rate of 30% or more, 35% or more, 40% or more, 45% or more, 50% or more, 55% or more, 60% or more, 65% or more, 70% or more, 75% or more, 80% or more, 85% or more, or 90% or more, for the cell type-specific or cell-subtype-specific membrane marker.

In some embodiments, the antibody has a specificity of 75% or more, 80% or more, 85% or more, 90% or more, 92% or more, 95% or more, 97% or more, or 99% or more for the cell type-specific or cell-subtype-specific membrane marker.

In some embodiments, the antibody has a capture rate of 30% or more, 35% or more, 40% or more, 45% or more, 50% or more, 55% or more, 60% or more, 65% or more, 70% or more, 75% or more, 80% or more, 85% or more, or 90% or more, for the cell type-specific or cell-subtype-specific membrane marker; and the antibody has a specificity of 85% or more, 90% or more, 92% or more, 95% or more, 97% or more, or 99% or more for the cell type-specific or cell-subtype-specific membrane marker.

In some embodiments, the antibody has a capture rate of 30% or more, 35% or more, 40% or more, 45% or more, for the cell type-specific or cell-subtype-specific membrane marker; and the antibody has a specificity of 85% or more, 90% or more, 92% or more, 95% or more, 97% or more, or 99% or more for the cell type-specific or cell-subtype-specific membrane marker.

In some embodiments, the antibody has a capture rate of 40% or more, 45% or more, 45% or more, 50% or more, for the cell type-specific or cell-subtype-specific membrane marker; and the antibody has a specificity of 92% or more, 95% or more, 97% or more, or 99% or more for the cell type-specific or cell-subtype-specific membrane marker.

In some embodiments, step (b) comprises selecting two cell type-specific or cell-subtype-specific membrane markers present on the surface of the exosomes to be isolated, and optionally wherein the immune-isolation of step (d) comprises simultaneous or sequential immune-isolation using the antibodies against respective two cell type-specific or cell-subtype-specific membrane markers present on the surface of the exosomes to be isolated.

In some embodiments, step (b) comprises:
generating or retrieving a list of membrane proteins of said mammal species, and/or
generating or retrieving a list of proteins present or enriched in the cell type or cell subtype of said mammal species, and/or
where the biological sample comprises a body fluid or is derived from a body fluid from a mammal, generating or retrieving a list of proteins present or enriched in the body fluid of said mammal species, and/or
generating or retrieving a list of cell type-specific or cell-subtype-specific membrane exosome proteins of said mammal species,
   and step (b) comprises selecting a protein present on two, three or four of these lists.

In some embodiments, step (b) comprises:
generating or retrieving a list of membrane proteins of said mammal species,
generating or retrieving a list of proteins present or enriched in the cell type or cell subtype of said mammal species,
where the biological sample comprises a body fluid or is derived from a body fluid from a mammal, generating or retrieving a list of proteins present or enriched in the body fluid of said mammal species, and
generating or retrieving a list of cell type-specific or cell-subtype-specific membrane exosome proteins of said mammal species,
   and step (b) further comprises selecting a protein present on all four of these lists.

In some embodiments, the one or more cell type comprises cells derived from the endoderm, cells derived from the mesoderm, or cells derived from the ectoderm.

In some embodiments, cells derived from the endoderm comprise cells of the respiratory system, the intestine, the liver, the gallbladder, the pancreas, the islets of Langerhans, the thyroid or the hindgut.

In some embodiments, cells derived from the mesoderm comprise osteochondroprogenitor cells, muscle cells, cells from the digestive systems, renal stem cells, cells from the reproductive system, bloods cells or cells from the circulatory system (such as endothelial cells).

In some embodiments, cells derived from the ectoderm, comprise epithelial cells, cells of the anterior pituitary, cells of the peripheral nervous system, cells of the neuroendocrine system, cell of the teethes, cell of the eyes, cells of the central nervous system, cells of the ependymal or cells of the pineal gland.

In some embodiments, cells from the central nervous system and the peripheral nervous system comprise neurons, Schwann cells, satellite glial cells, oligodendrocytes or astrocytes.

In some embodiments, neurons comprise interneurons, pyramidal neurons, gabaergic neurons, dopaminergic neurons, serotoninergic neurons, glutamatergic neurons, motor neurons from the spinal cord, or inhibitory spinal neurons.

In some embodiments, the one or more cell-type is a cancer cell or a circulating tumor cell (CTC), such as cancer cell or CTC derived from any cell-types or cell subtypes as defined herein.

In some embodiments, the antibody is immobilized on a solid substrate.

In some embodiments, the solid substrate is selected from a purification column, a microfluidic channel or beads, such as magnetic beads, magnetic nucleic acid binding beads, or silica beads functionalized with silane, for example Dynabeads® MyOne Silane Beads from Thermo Fisher Scientific.

In some embodiments, the immuno-isolation comprises a microfluidic affinity based isolation, a magnetic based isolation, a pull-down isolation or a fluorescence activated sorting-based isolation.

In some embodiments, the microfluidic channel is part of a system or device as described in Macosko E Z et al, Cell. 2015 May 21; 161(5):1202-1214. doi: 10.1016/j.cell.2015.05.002. Highly Parallel Genome-wide Expression Profiling of Individual Cells Using Nanoliter Droplets; in Klein A M et al, Cell. 2015 May 21; 161(5):1187-1201. doi: 10.1016/j.cell.2015.04.044. Droplet barcoding for single-cell transcriptomics applied to embryonic stem cells; and/or in WO2016040476.

In some embodiments, the biological sample comprises a body fluid or is derived from a body fluid, wherein the body fluid was obtained from a mammal.

In some embodiments, the body fluid is selected from amniotic fluid, aqueous humor, vitreous humor, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof

In some embodiments, step (b) comprises:
generating or retrieving a list of membrane proteins of said mammal species,
generating or retrieving a list of proteins present or enriched in a neural tissue cell type or cell subtype of said mammal species,
where the biological sample comprises cerebrospinal fluid or is derived from cerebrospinal fluid from a mammal, generating or retrieving a list of proteins present or enriched in cerebrospinal of said mammal species, and
generating or retrieving a list of cell type-specific or cell-subtype-specific membrane exosome proteins of said mammal species,
   and step (b) further comprises selecting a protein present on all of these lists.

In some embodiments, said cell type is selected from neurons, Schwann cells, satellite glial cells, oligodendrocytes or astrocytes.

In some embodiments, said cell type is selected from neurons and wherein said cell subtype is selected from interneurons, pyramidal neurons, gabaergic neurons, dopaminergic neurons, serotoninergic neurons, glutamatergic neurons, motor neurons from the spinal cord, or inhibitory spinal neurons.

In some embodiments, the one or more cell type-specific or cell-subtype-specific membrane marker(s) present on the surface of the exosomes to be isolated comprises L1CAM, CACNA2D1 or SYT1.

In one aspect, the invention relates to a method for the preparation of exosomal RNA from a biological sample, said method comprising:
(i) providing a biological sample comprising exosomes from a cell population,
(ii) isolating cell type-specific exosomes or cell-subtype-specific exosomes from the biological sample of step (i), in accordance with the method as disclosed herein, or performing immuno-isolation of exosomes from the biological sample of step (i) using the antibody or antibodies selected according to the method as disclosed herein,
(iii) extracting RNA from the isolated exosomes of step (ii).

In some embodiments, the exosomal RNA is total exosomal RNA.

In some embodiments, the exosomal RNA comprises exosomal messenger RNA.

In some embodiments, the exosomal RNA is total exosomal messenger RNA.

In one aspect, the presneti invention relates to a method for the determination of cellular RNA content in a cell population, said method comprising:

(A) providing a biological sample comprising exosomes from said cell population,

(B) isolating cell type-specific exosomes or cell-subtype-specific exosomes from the biological sample of step (A), in accordance with the method as described herein, or performing immuno-isolation of exosomes from the biological sample of step (A) using the antibody or antibodies selected according to the method as described herein,

(C) extracting RNA from the isolated exosomes of step (B), so as to provide exosomal RNA,

(D) analyzing the exosomal RNA extracted at step (C),

(E) estimating, as a function of the result from step (D), the cellular RNA content in the cell population.

In some embodiments, step (E) is performed based on a predicted correlation between exosomal RNA content and cellular RNA content.

In some embodiments, said determination comprises a qualitative determination.

In some embodiments, said determination comprises a quantitative determination.

In some embodiments, said quantitative determination comprises determination of relative abundance of two RNAs.

In some embodiments, said determination comprises determination of mRNA profiles.

In some embodiments, said RNA comprises messenger RNA (mRNA).

In some embodiments, said RNA comprises micro RNA (miRNA) or long non-coding RNA (lncRNA).

In some embodiments, step (D) comprises a qualitative determination, RNA sequencing (RNA seq), array analysis, reverse transcription polymerase chain reaction (RT-PCR), quantitative reverse transcription polymerase chain reaction (qRT-PCR).

In some embodiments, step (D) comprises analyzing one or more sequence/s of interest.

In some embodiments, the metohod comprises testing for the presence or absence of said sequence/s of interest, analyzing for one or more allelic variants of a sequence of interest, testing for presence or absence of said allelic variants.

In some embodiments, step (D) comprises genome-wide analysis.

In some embodiments, step (D) comprises transcriptome profiling.

In some embodiments, the determination is time-lapse.

In some embodiments, the method is for use in diagnosis.

In some embodiments, the method is for use in prognosis.

In some embodiments, the method is for use in a screening process.

In some embodiments, the method determines the cellular RNA content of a single cell type or of a single cell subtype.

In one aspec, the present invention relates to a method for the diagnostic or prognostic of a disorder of interest in a subject, comprising:
(I) selecting a biomarker, wherein said biomarker is associated with said disorder and wherein said biomarker may be determined in a cell type that is found in the subject to be in contact with a body fluid,
(II) providing a biological sample from said body fluid from said subject,
(III) estimating the cellular RNA content of said biomarker in the biological sample of step (II) by performing the method as described herein.

In some embodiments, the cellular RNA content is the cellular content of a single cell type or of a single cell subtype.

In some embodiments, the method further comprises (IV) determining, from the results of step (III), the status of the biomarker selected at step (I).

In some embodiments, the biomarker is selected from expression of a given open reading frame (ORF), overexpression of a given open reading frame (ORF), repression of a given open reading frame (ORF), over-repression of a given open reading frame (ORF), expression of a given allelic variant, relative level of expression of a given open reading frame (ORF), presence of a mutation in a given open reading frame (ORF).

In some embodiments, said disorder is a blood disorder and said biomarker is a biomarker that may be determined in one or more cell type/s that is/are found in the subject to be in contact with blood.

In some embodiments, in said disorder is a brain or spine disorder and said biomarker is a biomarker that may be determined in one or more cell type/s that is/are found in the subject to be in contact with cerebrospinal fluid.

In some embodiments, said disorder is a heart disorder and said biomarker is a biomarker that may be determined in one or more cell type/s that is/are found in the subject to be in contact with blood or pericardial fluid.

In some embodiments, said disorder is a prostate or bladder disorder and said biomarker is a biomarker that may be determined in one or more cell type/s that is/are found in the subject to be in contact with urine.

In some embodiments, said disorder is an eye disorder and said biomarker is a biomarker that may be determined in one or more cell type/s that is/are found in the subject to be in contact with tears.

In some embodiments, said disorder is a lung disorder and said biomarker is a biomarker that may be determined in one or more cell type/s that is/are found in the subject to be in contact with pleural fluid.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

The following detailed description, given by way of example, but not intended to limit the invention solely to the specific embodiments described, may best be understood in conjunction with the accompanying drawings of which:

FIG. 1 shows graph of RNA fluorescence unit (FU) plotted against RNA size (nt) for various exosome purification methods.

FIGS. 2A-2D show electron microscopy (EM) photographs of exosome preparations for various exosome purification methods;(2A) Electron microscopy of exosomes with no treatment; (2B) Electron microscopy of exosomes with proteinase treated after spins; (2C) Side-by-side comparison of EM of untreated versus proteinase-treated; (2D) Electron microscopy of exosomes with proteinase treated between spins.

FIG. 3 shows results of a qRT-PCR experiment for various exosome purification methods.

FIG. 4A-4C show RNA-Seq data, showing that the RNA profile of mRNAs in exosomes reflects that of the donor cells; (4A) illustrates mRNA profile in exosomes: PTMS; (4B) illustrates mRNA profile in exosomes: MT2A; (4C) illustrates mRNA profile in exosomes: Rab13.

FIGS. 5A-5K show principle and results for fluorescence imaging of cells using EU click chemistry, to assess possible exosome-mediated RNA transfer between cells; (5A) shows intercellular communication (5B) shows click-chemistry with 5-ethynyl uridine (5C) shows control HEK 293 cells grown in presence of 5-ethynyl uridine; (5D) shows negative control of HEK 293 cells with no 5-ethynyl uridine; (5E) illustrates RNA transfer experiment; (5F) shows negative control of HEK 293/K562 cells with no 5-ethynyl uridine (5G) shows negative control of HEK 293/K562 cells with no 5-ethynyl uridine with 640× magnification zoomed in; (5H) shows experimental #1 of HEK 293/K562 cells with 5-ethynyl uridine (5I) shows experimental #1 of HEK 293/K562 cells with 5-ethynyl uridine (6J) shows experimental #1 of HEK 293/K562 cells with 5-ethynyl uridine (zoomed in); (5K) shows experimental #2 of HEK 293/K562 cells with 5-ethynyl uridine.

FIGS. 6A-6D show principle and results of an experiment to assess possible exosome mediated RNA transfer between co-cultured cell lines; (6A) illustrates an alternative experiment of mouse-human co-culture; (6B) shows the experimental design; (6C) percentage of mouse genes with TMM>2; (6D) shows mouse gene expression in human cells.

FIGS. 7A-7D illustrates Poly A selected from mRNA from two replicates of K562 cells and their exosomes was compared using RNA-Seq; (7A) compares cell 1 versus cell 2; (7B) compares exosome 1 versus exosome 2; (7C) compares cell 1 versus exosome 1 (7D) compares cell 2 versus exosome 2.

FIG. 8 illustrates that mRNA is inside the exosomes.

FIG. 9 illustrates Poly A enriched mRNA from untreated exosomes and proteinase/Rnase treated exosomes was compared using RNA-Seq.

FIG. 10 illustrates targeted pull down exosome subpopulations based on their protein marker using antibody conjugated magnetic beads.

FIG. 11 illustrates exosomes which were isolated from human CSF and mRNA for four genes (detected by qRT-PCR.) Cell RNA is used as a comparison.

FIGS. 12-76 provide a series of schematics and Western blot showing selection of candidate exosome targets and optimization of isolation methods.

FIG. 77 provides results of a qRT-PCR experiments. 10 pg or 100 pg of purified RNA from K562 cells were used alongside three samples: RNA from K562 cells, RNA from K562 exosomes and RNA from a CD83 pulldown. qRT-PRC was performed for two mRNAs to quantify the relative amounts of RNA.

FIG. 78 provides a graph showing total exosomes from CSF were isolated and transcripts from neuron-specific genes are detected.

### DETAILED DESCRIPTION OF THE INVENTION

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboraotry Manual (1988) (Harlow and Lane, eds.): Antibodies A Laboraotry Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by referenceThe terms “exosomes”, “micro-vesicles” and “extracellular vesicles” are herein used interchangeably. They refer to extracellular vesicles, which are generally of between 30 and 200 nm, for example in the range of 50-100 nm in size. In some aspects, the extracellular vesicles can be in the range of 20-300 nm in size, for example 30-250 nm in size, for example 50-200 nm in size. In some aspects, the extracellular vesicles are defined by a lipidic bilayer membrane.

### Overview

In aspects of the invention functional genomics screens allow for discovery of novel human and mammalian therapeutic applications, including the discovery of novel drugs, for, e.g., treatment of genetic diseases, cancer, fungal, protozoal, bacterial, and viral infection, ischemia, vascular disease, arthritis, immunological disorders, etc. As used herein assay systems may be used for a readout of cell state or changes in phenotype include, e.g., transformation assays, e.g., changes in proliferation, anchorage dependence, growth factor dependence, foci formation, growth in soft agar, tumor proliferation in nude mice, and tumor vascularization in nude mice; apoptosis assays, e.g., DNA laddering and cell death, expression of genes involved in apoptosis; signal transduction assays, e.g., changes in intracellular calcium, cAMP, cGMP, IP3, changes in hormone and neurotransmittor release; receptor assays, e.g., estrogen receptor and cell growth; growth factor assays, e.g., EPO, hypoxia and erythrocyte colony forming units assays; enzyme product assays, e.g., FAD-2 induced oil desaturation; transcription assays, e.g., reporter gene assays; and protein production assays, e.g., VEGF ELISAs.

In the purification methods of the invention, it was found advantageous to perform a proteinase treatment, especially after the final ultracentrifugation step carried out for exosome preparation. Without being bound by theory, it is hypothesized that such treatment allows the removal of non exosomal nucleic acid-protein complexes, such as RNA-protein complexes. The proteinase treatment (and inactivation thereof), may then be followed by an RNAse treatment.

The exosome purification and isolation methods of the invention allows one to prepare compositions comprising exosomes, wherein the composition is essentially free of extra-exosomal material, and/or essentially free of extra-exosomal nucleic acid-protein complexes, and/or essentially free of extra-exosomal RNA-protein complexes. Such compositions may be used for exosomal RNA preparation.

The purification or isolation method of the invention may include the following: removal of live cells, dead cells and larger cell debris, which may be performed by centrifugation steps and collection of the corresponding supernatants; filtration using a submicron filter such as a 0.22 micron filter; collection of exosomes by ultracentrifugation (typically at 100 g-130,000 g, for example 120,000 g); washing exosomes before an additional ultracentrifugation step; proteinase treatment and inactivation; RNase treatment and inactivation.

According to one aspect of the invention, a strong correlation can advantageously be established between the RNA profile, and notably the mRNA profile, of isolated or purified exosomes and the RNA profile of the corresponding donor cells. In particular, a correlation has been shown between the mRNA profile of exosomes from K562 cells which have been isolated or purified as per the purification method or the invention, notably after treatment with protease and then RNAse, and the RNA profile of donor K562 cells. Such a correlation has been shown for the first time and is advantageous for diagnostic applications, as the transcriptome profile from exosomes of a cell population very faithfully reflects the corresponding cellular transcriptome.

Furthermore, a correlation can also be established between the RNA content (notably the mRNA content) of purified or isolated exosomes treated with protease and RNase and the RNA content of protease/RNAse untreated exosomes. These results illustrate that the analyzed RNA content of exosomes isolated or purified as per the purification method of the invention is actually inside said exosomes and not simply externally associated with exosomes. Analyses of the RNA exosomal content can be performed using any transcriptomics method (see notably Wang et.al, Nature Review Genetics (10) 57-63), such as RNA seq (for which a princeps protocol is notably described in Macosko E Z et al., 2015, Cell 161, 1202-1214), RT-PCT (notably qRP-PCR), small RNA sequencing (Li et.al, NAR 41(6) 3619-3634) or microarray. RNA analysis can also be performed as described in “Chip-based analysis of exosomal mRNA mediating drug resistance in glioblastoma”. Shao H, Chung J, Lee K, Balaj L, Min C, Carter B S, Hochberg F H, Breakefield X O, Lee H, Weissleder R. Nat Commun. 2015 May 11; 6:6999. doi: 10.1038/ncomms7999. PMID: 25959588; “Microfluidic isolation and transcriptome analysis of serum microvesicles”. Chen C, Skog J, Hsu C H, Lessard R T, Balaj L, Wurdinger T, Carter B S, Breakefield X O, Toner M, Irimia D. Lab Chip. 2010 Feb. 21; 10(4):505-11. doi: 10.1039/b916199f. Epub 2009 Dec. 8. PMID: 20126692.

In some aspects, the purification method of the invention may further comprise a step of separating one or more sub-populations of exosomes from a purified pool of exosomes. Indeed in some aspects of the invention, a sub-population of exosomes from a mixed exosome population, found for example in a biological sample obtained from a body fluid, can be further purified or isolated, for example according to one or more specific donor cell types or donor cell subtypes. In some aspects, the purification method of the invention allows to isolate or purify subpopulations of exosomes from one or more cell types or cell subtypes, preferentially from a single cell type, or from a single cell subtype.

In some aspects, a cell population can comprise one or more cell types, notably 2 or more cell types, 3 or more cell types, 4 or more cell types, or 5 or more cell types. In some aspects, a cell population comprises at least 1 to 40 cell types, notably at least 1 to 30, at least 5 to 20, at least 5 to 10, at least 2 to 8 or at least 2 to 5 cell types. Therefore, cell type or cell subtype exosomes can be purified from a mixed exosome population obtained from a cell population.

In some aspects, cell types according to the invention comprises cell types derived from the endoderm, cell types derived from the mesoderm, or cell types derived from the ectoderm. Cell types derived from the endoderm can comprise cell types of the respiratory system, the intestine, the liver, the gallbladder, the pancreas, the islets of Langerhans, the thyroid or the hindgut. Cell types derived from the mesoderm can comprise osteochondroprogenitor cells, muscle cells, cell types from the digestive system, renal stem cells, cell types from the reproductive system, bloods cell types or cell types from the circulatory system (such as endothelial cells). Cell types derived from the ectoderm can comprise epithelial cells, cell types of the anterior pituitary, cell types of the peripheral nervous system, cell types of the neuroendocrine system, cell types of the teeth, cell types of the eyes, cell types of the central nervous system, cell types of the ependymal or cell types of the pineal gland. For example, a cell population from the central and peripheral nervous system can comprise cell types such as neurons, Schwann cells, satellite glial cells, oligodendrocytes or astrocytes. In some aspects of the invention, the one or more cell types comprise cancer cells or circulating tumor cells. Preferentially, said cancer cells or CTCs derive from the cell types as listed above. A cell type can also encompass one or more cell subtypes, notably 2 or more, 3 or more, 4 or more, 5 or more and up to 10 or more cell subtypes. For example neurons encompass various cell subtypes such as for example interneurons, pyramidal neurons, gabaergic neurons, dopaminergic neurons, serotoninergic neurons, glutamatergic neurons, motor neurons from the spinal cord, or inhibitory spinal neurons. Different cell types or cell subtypes can also be discriminated according to their respective transcriptome profile.

In some aspects, purification (isolation) of exosomes according to a specific cell type or a cell subtype is achieved through one or more purification or isolation steps. Isolation can beperformed using one one or more antibody against each of the one or more the cell type-specific or cell-subtype-specific membrane marker(s).

Exosome biomarkers (cell type-specific or cell-subtype-specific membrane marker(s)) can be typically identified through mass spectrometry analyses of exosomes obtained from specific cell types or cell subtypes, and if required confirmed through western blotting or qRT-PCR analysis in said exosomes. For example exosomes from induced pluripotent stem cells (IPS cells) or IPS-derived- neurons can be used, but exosomes from any cell types or cell subtypes as defined above can be subjected to mass spectrometry analysis for identification of specific trans-membrane protein biomarkers. For example, mass spectrometry analysis can also be performed on total exosomes from a body fluid, such as CSF. Analysis of the transcriptome of CSF exosomes is of high interest because such exosome population is specific of the brain cell population.

Data obtained from such mass spectrometry analysis can be combined with genome or transcriptome analysis of corresponding donor cells in order to identify relevant biomarkers. This facilitates the identification of relevant exosome biomarkers useful for the present invention. For example, regarding CNS genetic information, lists of genes are available from e.g. “Establishing the Proteome of Normal Human Cerebrospinal Fluid” Schutzer S E et al., PLoS One, 2010; 5(6): e10980. “An RNA-Sequencing Transcriptome and Splicing Database of Glia, Neurons, and Vascular Cells of the Cerebral Cortex” Zhang Y et al., The Journal of Neuroscience, 2014, 34(36):11929-11947. “Purification and Characterization of Progenitor and Mature Human Astrocytes Reveals Transcriptional and Functional Differences with Mouse” Zhang et al., 2016, Neuron 89, 37-53.

The presence of the at least one of these trans-membrane protein biomarkers in neuron exosomes can be confirmed through western blotting or RT-PCT analysis or neuron exosomes.

More generally, cell type-specific or cell-subtype-specific membrane marker(s) can be identified by combining several lists of candidates, based on a list of membrane proteins of said mammal species, and/or a list of proteins present or enriched in the cell type or cell subtype of said mammal species, and/or where the biological sample comprises a body fluid or is derived from a body fluid from a mammal, a list of proteins present or enriched in the body fluid of said mammal species, and/or a list of cell type-specific or cell-subtype-specific membrane exosome proteins of said mammal species.

For example, data from the Human Cell Atlas (https://www.humancellatlas.org/), or other genomics, transcriptomics or proteomics data from available literature. In some aspects, single cell data can also be used.

For the lists used herein, it is alos possible to refer to other species (e.g. another mammal species), for example using data from the mouse, the rabbit ro the monkey.

“Dysfunctionally phosphorylated type 1 insulin receptor substrate in neural-derived blood exosomes of preclinical Alzheimer's disease”. Kapogiannis D, Boxer A, Schwartz J B, Abner E L, Biragyn A, Masharani U, Frassetto L, Petersen R C, Miller B L, Goetzl E J. FASEB J. 2015 February; 29(2):589-96. doi: 10.1096/fj.14-262048. Epub 2014 Oct. 23. PMID: 25342129 and “Plasma exosomal α-synuclein is likely CNS-derived and increased in Parkinson's disease”. Shi M, Liu C, Cook T J, Bullock K M, Zhao Y, Ginghina C, Li Y, Aro P, Dator R, He C, Hipp M J, Zabetian C P, Peskind E R, Hu S C, Quinn J F, Galasko D R, Banks W A, Zhang J. Acta Neuropathol. 2014 November; 128(5):639-50. doi: 10.1007/s00401-014-1314-y. Epub 2014 Jul. 6. PMID: 24997849 describe analysis of exosomes obtained from plasma, but as such do not provide informative or conclusive evidence establishing a relationship with a specific organ of origin (such as brain) or specific tissue of origin or a fortiori specific cell types of origin such as neurons. This is because of the circulating nature of plasma that comes into contact with a number of various organs, tissues, etc., and thus may comprise exosomes stemming from a plurality of different cell types altogether. Further, it is unclear whether some exosomes are capable of corrsing the blood brain barrier. As a consequence, the data reported in these papers do not allow to identify the exact origin of the exosomes, and in particular cannot relate to exosomes from a specific cell type (such as neurons). Further, these papers do not disclose any RNA profiling, in particular, no RNA-seq analysis.

By contrast, the present invention provides methods for accessing information on tissue- or cell-type-specific exosomes, in particular tissue- or cell-type-specific transcription profiles. The present invention also provides very-high resolution diagnostic methods, wherein a subtle change in transcription profiles (e.g. a small up- or down-regulation in the transcription of a given gene in a given cell type or a given cell sub-type) can advantageously be efficiently detected, while it could not be in a total RNA or total exosome analysis.

In some aspects the one or more purification steps can comprise a microfluidic affinity based purification (see for example “Chip-based analysis of exosomal mRNA mediating drug resistance in glioblastoma”. Shao H, Chung J, Lee K, Balaj L, Min C, Carter B S, Hochberg F H, Breakefield X O, Lee H, Weissleder R. Nat Commun. 2015 May 11; 6:6999. doi: 10.1038/ncomms7999. PMID: 25959588; “Microfluidic isolation and transcriptome analysis of serum microvesicles”. Chen C, Skog J, Hsu C H, Lessard R T, Balaj L, Wurdinger T, Carter B S, Breakefield X O, Toner M, Irimia D. Lab Chip. 2010 Feb. 21; 10(4):505-11. doi: 10.1039/b916199f. Epub 2009 Dec. 8. PMID: 20126692.), a magnetic based purification, a pull-down purification or a fluorescence activated vesicle sorting-based purification (FAVS, see for example Van der Pol E et al., J Thromb Haemost., 2013 June; 11 Suppl 1:36-45 “Innovation in detection of microparticles and exosomes” and Van des Pol E. et al., J Thromb Haemost. 2012 May; 10(5):919-30), “Single vs. swarm detection of microparticles and exosomes by flow cytometry”; “Glypican-1 identifies cancer exosomes and detects early pancreatic cancer”. Melo S A, Luecke L B, Kahlert C, Fernandez A F, Gammon S T, Kaye J, LeBleu V S, Mittendorf E A, Weitz J, Rahbari N, Reissfelder C, Pilarsky C, Fraga M F, Piwnica-Worms D, Kalluri R. Nature. 2015 Jul. 9; 523(7559):177-82. doi: 10.1038/nature14581. Epub 2015 Jun. 24. PMID: 26106858). Commercial precipitation kits like ExoQuick™ and Total Exosome Isolation™ precipitation solutions are also available. Such kits are easy to use with only 1 or 2 steps and do not require any expensive equipment or advanced technical know-how.

Immune-isolation can be performed using a bait/prey strategy.

In some aspects, the bait molecule can be a bait protein, such as an antibody and in some aspects is preferentially a monoclonal antibody directed against a prey exosome biomarker (antibody against each of the one or more the cell type-specific or cell-subtype-specific membrane marker(s)). In some aspects, the bait molecule can also be an RNA aptamer. If several prey exosomes are to be combined for purification, a mix of corresponding monoclonal antibodies directed against each of the said prey exosomes biomarkers to be pull-up can be used.

In some aspects, the bait molecule is recognized by an affinity ligand. Said affinity ligand can be a divalent metal-based complex, a protein, a peptide such as fusion protein tag or more preferentially an antibody.

In some aspects, the bait molecule or the affinity ligand is immobilized or “coupled” directly, or indirectly to a solid substrate material such as by formation of covalent chemical bonds between particular functional groups on the ligand (for example primary amines, thiols, carboxylic acids, aldehydes) and reactive groups on the substrate. A substrate, or a matrix, in the affinity purification steps of the method of the invention can be any material to which a biospecific ligand (i.e., the bait molecule or the affinity ligand) is coupled. Useful affinity supports may be those with a high surface-area to volume ratio, chemical groups that are easily modified for covalent attachment of ligands, minimal nonspecific binding properties, good flow characteristics and/or mechanical and chemical stability. Several substrates may be utilized as solid substrate, including for example agarose, cellulose, dextran, polyacrylamide, latex or controlled pore glass. Magnetic particles may also be used as a substrate instead of beaded agarose or other porous resins. Their small size provides the sufficient surface area-to-volume ratio needed for effective ligand immobilization and affinity purification. Magnetic beads may be produced as superparamagnetic iron oxide particles that may be covalently coated with silane derivatives. The coating makes the beads inert (i.e., to minimize nonspecific binding) and provides the particular chemical groups needed for attaching any affinity ligands of interest. Affinity purification with magnetic particles is generally not performed in-column. Instead, a few microliters of beads may be mixed with several hundred microliters of sample as a loose slurry. During mixing, the beads remain suspended in the sample solution, allowing affinity interactions to occur with the immobilized ligand. After sufficient time for binding has been given, the beads are collected and separated from the sample using a powerful magnet. An exemplary bead purification method can be found in “Proteomic comparison defines novel markers to characterize heterogeneous populations of extracellular vesicle subtypes”. Kowal J, Arras G, Colombo M, Jouve M, Morath J P, Primdal-Bengtson B, Dingli F, Loew D, Tkach M, Thery C. Proc Natl Acad Sci USA. 2016 Feb. 23; 113(8):E968-77. doi: 10.1073/pnas.1521230113. Epub 2016 Feb. 8. PMID: 26858453.

In some aspects of the invention, a pull down assay can be performed for the purification or isolation of a subpopulation of exosomes by pulling-down of one or more specific prey exosome biomarkers (preferentially a membrane protein as described below), e.g. using one or more antibody against each of the one or more the cell type-specific or cell-subtype-specific membrane marker(s). Said prey exosome biomarkers may be specific of a at least one cell type or cell subtype and advantageously lead to enriching in exosomes from said selected cell type or cell subtype.

In some aspects the at least one or more purification steps for the purification of an exosome subpopulation comprise a pull down purification. In such pull-down purification, the prey exosome biomarker is generally a (trans)membrane protein, which has been found to be expressed in a cell type or a cell subtype. The bait protein is preferentially a monocolonal antibody directed against any of the prey exosome biomarker(s) which is to be pulled-up. Magnetic beads such as magnetic nucleic acid binding beads, or silica beads functionalized with silane (for example Dynabeads® from Thermo Fisher Scientific, such as Dynabeads® MyOne Silane Beads from Thermo Fisher Scientific) coated with an affinity ligand for the bait protein can be used to isolate said bait protein bound to said prey exosome biomarker(s). The affinity ligand is preferentially a class specific or a species specific antibody. As a matter of example, magnetic beads coated with anti-mouse antibodies can be used together with monoclonal mouse antibodies directed against a specific surface protein of a cell type or cell subtype subpopulation of exosomes (such as for example CD63 or CD81). Generally, a control antibody, such as a mouse mcherry monoclonal antibody, can be used.

A pull down assay can therefore be used to illustrate and validate the purification, or isolation of at least two exosome subpopulations expressing each at least one specific membrane protein, such as the canonical exosomes markers CD63 and CD81, which have previously been pooled. As shown in the results examples, said at least two exosomes subpopulations can be re-separated based on the selected protein biomarker. The purification or isolation of exosome subpopulations by at least one specific prey exosome biomarker (preferentially a membrane protein) can be further confirmed using wertern blot or qRT-PCT.

The present invention relates to selecting one or more cell type-specific or cell-subtype-specific membrane marker(s) present on the surface of the exosomes to be isolated,

selecting an antibody against each of the one or more the cell type-specific or cell-subtype-specific membrane marker(s), wherein said antibody (resp. each of said antibodies) has (have):
a capture rate of 30% or more for the cell type-specific or cell-subtype-specific membrane marker, and
a specificity of 70% or more for the cell type-specific or cell-subtype-specific membrane marker.

The combination of such capture rate values and specificity values are very advantageous for the isolation of cell type-specific exosomes or cell-subtype-specific exosomes from a biological sample.

Capture rate generally indicates the recovery of relevant exosomes using the isolation when comparing to amounts found in the un-isolated fraction (flow through, unbound, . . . )) to the amounts found in the isolated fraction (pull-down, bound, . . . ).

Specificity generally reflects on the performance of the antibody against each of the one or more the cell type-specific or cell-subtype-specific membrane marker(s) for unspecific binding, and can be assessed using a non-specific antibody, such as anti-GFP or another control antibody.

In some embodiments, the antibody has a capture rate of 30% or more, 35% or more, 40% or more, 45% or more, 50% or more, 55% or more, 60% or more, 65% or more, 70% or more, 75% or more, 80% or more, 85% or more, or 90% or more, for the cell type-specific or cell-subtype-specific membrane marker.

In some embodiments, the antibody has a specificity of 75% or more, 80% or more, 85% or more, 90% or more, 92% or more, 95% or more, 97% or more, or 99% or more for the cell type-specific or cell-subtype-specific membrane marker.

In some embodiments, the antibody has a capture rate of 30% or more, 35% or more, 40% or more, 45% or more, 50% or more, 55% or more, 60% or more, 65% or more, 70% or more, 75% or more, 80% or more, 85% or more, or 90% or more, for the cell type-specific or cell-subtype-specific membrane marker; and the antibody has a specificity of 85% or more, 90% or more, 92% or more, 95% or more, 97% or more, or 99% or more for the cell type-specific or cell-subtype-specific membrane marker.

In some embodiments, the antibody has a capture rate of 30% or more, 35% or more, 40% or more, 45% or more, for the cell type-specific or cell-subtype-specific membrane marker; and the antibody has a specificity of 85% or more, 90% or more, 92% or more, 95% or more, 97% or more, or 99% or more for the cell type-specific or cell-subtype-specific membrane marker.

In some embodiments, the antibody has a capture rate of 40% or more, 45% or more, 45% or more, 50% or more, for the cell type-specific or cell-subtype-specific membrane marker; and the antibody has a specificity of 92% or more, 95% or more, 97% or more, or 99% or more for the cell type-specific or cell-subtype-specific membrane marker.

Those of skill in the art are aware of parameters that can influence isolation performance. For example, for a bead-based pull-down, various experimental conditions can be used. Examples of parameters in a bead pull down include:
Nature and composition of the buffer, including nature and concentration of salt/s, presence and concentration of BSA, nature and concentration of detergent/s,
Volume of the reaction
Time duration of the binding reaction,
Temperature of the binding reaction,
antibody clone,
selection of the beads, including the selection of the anti-IgG, nature of the beads, including option for epoxylated or tosylactivated
bead/antibody ratio,
bead/exosome ratio,
flow-through recovery,
conditions for the washes or elution strategy
immunoblotting parameters
etc

Several control experiments can also be envisioned to compare the transcriptome of subpopulation of exosomes, purified or isolated by pull-up of at least one specific exosome biomarker, according to the method of the invention.
It is advantageously possible to compare the transcriptome profile of at least two subpopulations of exosomes, purified from a mixed exosome population (e.g.: obtained from a cell population comprising one or more cell types, such as the K562 cells) using specific exosome biomarkers (such as CD63 or CD81) as described above (e.g.: using magnetic beads pull-down purification). The transcriptome profile of said exosomes subpopulations can also be further compared to the transcriptome profile of the total exosome population. Typically RNA seq analysis of exosomes is particulary well suited for such transcriptome comparisons.
It is advantageously possible to compare the RNA seq analysis of total RNA, mRNA, micro RNA (miRNA), or long non coding RNA (lncRNA) of (i) at least one cell type and (ii) exosomes obtained from said at least one cell type. As a matter of example, it is possible to perform RNA seq analysis of mRNA from (i) IPS cells and IPS-derived neurons, and (ii) exosomes obtained respectively from said IPS cells and IPS-derived neurons and and then compare the obtained results.
It is advantageously possible to compare (i) transcriptome profile analysis (notably the

RNA seq analysis) of exosomes from the said different cell types or subtypes, isolated according through the purification method of the invention (notably using antibody-conjugated magnetic beads as described above) in order to enrich for exosomes expressing at least one cell type or cell subtype specific biomarker, with (ii) the transcriptome profile of total exosomes. For example the RNA seq results of exosomes from IPS cells and neuron exosomes isolated according to the pull down assay as described above can be compared to the RNA profile of total exosomes from both cell types.
In vitro experiments for the control of the purification of exosome subpopulations can also comprise experiments, wherein exosomes subspopulations are purified or isolated from a complex biological sample obtained from at least two cell populations, cell types, or cell subtypes. For example, from a mix of media obtained from cell culture of different cell types such as IPS cells and neurons. Exosomes of the specific cells types are then purified as described above and their transcriptome is analysed. Such an experiment allows reconstructing, ex post facto, the transcriptome of the original cell type.

Isolation or purification of total exosomes from biological samples derived from any body fluid such as CSF, urine, or blood etc. and transcriptome analysis of the obtained exosome population can also be envisioned. Using cell-type specific biomarkers, exosome subpopulations can be further purified through any of the purification steps as described above, and enrichment in expression of specific cell type biomarkers can be searched through transcriptome analysis of this subpopulation as compared to the total exosome population. Said analysis is of particular interest for CSF analysis and identification of exosomes from specific neuronal subtypes

According to the present invention, the RNA content of exosomes is found to correlate the RNA content of the corresponding cell. In other terms, in particular when exosomes are purified in accordance with purification method of the present invention, a correlation was found between said exosomal RNA content and corresponding cellular RNA content. Therefore, analyzing exosomal RNA provides both qualitative and quantitative information about the cellular RNA content of the corresponding cells. Advantageously, this makes it possible to provide non-invasive diagnostic methods. Indeed, the analysis (whether by RNA seq, transcriptome profiling, qRT-PCR or array) is performed on a biological sample derived from body fluids, such as derived from urine, blood or cerebrospinal fluid. Such fluids are more easily and readily available than corresponding organs (bladder, heart or brain). Correspondingly, the present invention provides diagnostic methods that are non-invasive and yet reliable. In some aspects, it is envisioned to use a subpopulation of exosomes as starting material to extract RNA. This may allow the analysis of exosome subpools/subpopulations.

Below are examples of lists that can be used to select one or more cell type-specific or cell-subtype-specific membrane marker(s) present on the surface of the exosomes to be isolated:

| | |
|---|---|
| Transmembrane proteins | “Establishing the Proteome of Normal |
| detected in cell-free | Human Cerebrospinal Fluid” Schutzer S E et |
| CSF through mass | al., PLoS One, 2010; 5(6): e10980. |
| spectrometry analysis. | This paper provides a list of proteins detected |
| | through mass spectrometry analysis in cell- |
| | free CSF. |
| Neuron specific genes | “An RNA-Sequencing Transcriptome and |
| expressed in mouse | Splicing Database of Glia, Neurons, and |
| | Vascular Cells of the Cerebral Cortex” |
| | Zhang Y et al., The Journal of Neuroscience, |
| | 2014, 34(36): 11929-11947. |
| | This paper compares gene expression in |
| | different cells of the brain in mouse |
| Neuron specific genes | “Purification and Characterization of |
| expressed in human | Progenitor and Mature Human Astrocytes |
| | Reveals Transcriptional and Functional |
| | Differences with Mouse” Zhang et al., 2016, |
| | Neuron 89, 37-53 |
| | This paper compares gene expression in |
| | different cells of the brain in human. |

The present invention may be applied to genetic mutations further described in Genetic Diseases of the Eye, Second Edition, edited by Elias I. Traboulsi, Oxford University Press, 2012. Several further aspects of the invention relate to diagnosing, prognosing and/or treating defects associated with a wide range of genetic diseases which are further described on the website of the National Institutes of Health under the topic subsection Genetic Disorders (website at health.nih.gov/topic/Genetic Disorders). The genetic brain diseases may include but are not limited to Adrenoleukodystrophy, Agenesis of the Corpus Callosum, Aicardi Syndrome, Alpers' Disease, Alzheimer's Disease, Barth Syndrome, Batten Disease, CADASIL, Cerebellar Degeneration, Fabry's Disease, Gerstmann-Straussler-Scheinker Disease, Huntington's Disease and other Triplet Repeat Disorders, Leigh's Disease, Lesch-Nyhan Syndrome, Menkes Disease, Mitochondrial Myopathies and NINDS Colpocephaly. These diseases are further described on the website of the National Institutes of Health under the subsection Genetic Brain Disorders.

In some embodiments, the condition (disorder) may be neoplasia. In some embodiments, where the condition is neoplasia, the genes to be diagnosed, prognosed and/or targeted are any of those listed in Table A (in this case PTEN and so forth). In some embodiments, the condition may be Age-related Macular Degeneration. In some embodiments, the condition may be a Schizophrenic Disorder. In some embodiments, the condition may be a Trinucleotide Repeat Disorder. In some embodiments, the condition may be Fragile X Syndrome. In some embodiments, the condition may be a Secretase Related Disorder. In some embodiments, the condition may be a Prion-related disorder. In some embodiments, the condition may be ALS. In some embodiments, the condition may be a drug addiction. In some embodiments, the condition may be Autism. In some embodiments, the condition may be Alzheimer's Disease. In some embodiments, the condition may be inflammation. In some embodiments, the condition may be Parkinson's Disease.

Examples of proteins associated with Parkinson's disease include but are not limited to α-synuclein, DJ-1, LRRK2, PINK1, Parkin, UCHL1, Synphilin-1, and NURR1.

Examples of addiction-related proteins may include ABAT for example.

Examples of inflammation-related proteins may include the monocyte chemoattractant protein-1 (MCP1) encoded by the Ccr2 gene, the C—C chemokine receptor type 5 (CCR5) encoded by the Ccr5 gene, the IgG receptor IIB (FCGR2b, also termed CD32) encoded by the Fcgr2b gene, or the Fc epsilon R1g (FCER1g) protein encoded by the Fcer1g gene, for example.

Examples of cardiovascular diseases associated proteins may include IL1B (interleukin 1, beta), XDH (xanthine dehydrogenase), TP53 (tumor protein p53), PTGIS (prostaglandin 12 (prostacyclin) synthase), MB (myoglobin), IL4 (interleukin 4), ANGPT1 (angiopoietin 1), ABCG8 (ATP-binding cassette, sub-family G (WHITE), member 8), or CTSK (cathepsin K), for example.

Examples of Alzheimer's disease associated proteins may include the very low density lipoprotein receptor protein (VLDLR) encoded by the VLDLR gene, the ubiquitin-like modifier activating enzyme 1 (UBA1) encoded by the UBA1 gene, or the NEDD8-activating enzyme E1 catalytic subunit protein (UBE1C) encoded by the UBA3 gene, for example.

Examples of proteins associated Autism Spectrum Disorder may include the benzodiazapine receptor (peripheral) associated protein 1 (BZRAP1) encoded by the BZRAP1 gene, the AF4/FMR2 family member 2 protein (AFF2) encoded by the AFF2 gene (also termed MFR2), the fragile X mental retardation autosomal homolog 1 protein (FXR1) encoded by the FXR1 gene, or the fragile X mental retardation autosomal homolog 2 protein (FXR2) encoded by the FXR2 gene, for example.

Examples of proteins associated Macular Degeneration may include the ATP-binding cassette, sub-family A (ABC1) member 4 protein (ABCA4) encoded by the ABCR gene, the apolipoprotein E protein (APOE) encoded by the APOE gene, or the chemokine (C—C motif) Ligand 2 protein (CCL2) encoded by the CCL2 gene, for example.

Examples of proteins associated Schizophrenia may include NRG1, ErbB4, CPLX1, TPH1, TPH2, NRXN1, GSK3A, BDNF, DISCI, GSK3B, and combinations thereof.

Examples of proteins involved in tumor suppression may include ATM (ataxia telangiectasia mutated), ATR (ataxia telangiectasia and Rad3 related), EGFR (epidermal growth factor receptor), ERBB2 (v-erb-b2 erythroblastic leukemia viral oncogene homolog 2), ERBB3 (v-erb-b2 erythroblastic leukemia viral oncogene homolog 3), ERBB4 (v-erb-b2 erythroblastic leukemia viral oncogene homolog 4), Notch 1, Notch2, Notch 3, or Notch 4, for example.

Examples of proteins associated with a secretase disorder may include PSENEN (presenilin enhancer 2 homolog (C. elegans)), CTSB (cathepsin B), PSEN1 (presenilin 1), APP (amyloid beta (A4) precursor protein), APH1B (anterior pharynx defective 1 homolog B (C. elegans)), PSEN2 (presenilin 2 (Alzheimer disease 4)), or BACE1 (beta-site APP-cleaving enzyme 1), for example.

Examples of proteins associated with Amyotrophic Lateral Sclerosis may include SOD1 (superoxide dismutase 1), ALS2 (amyotrophic lateral sclerosis 2), FUS (fused in sarcoma), TARDBP (TAR DNA binding protein), VAGFA (vascular endothelial growth factor A), VAGFB (vascular endothelial growth factor B), and VAGFC (vascular endothelial growth factor C), and any combination thereof.

Examples of proteins associated with prion diseases may include SOD1 (superoxide dismutase 1), ALS2 (amyotrophic lateral sclerosis 2), FUS (fused in sarcoma), TARDBP (TAR DNA binding protein), VAGFA (vascular endothelial growth factor A), VAGFB (vascular endothelial growth factor B), and VAGFC (vascular endothelial growth factor C), and any combination thereof.

Examples of proteins related to neurodegenerative conditions in prior disorders may include A2M (Alpha-2-Macroglobulin), AATF (Apoptosis antagonizing transcription factor), ACPP (Acid phosphatase prostate), ACTA2 (Actin alpha 2 smooth muscle aorta), ADAM22 (ADAM metallopeptidase domain), ADORA3 (Adenosine A3 receptor), or ADRA1D (Alpha-1D adrenergic receptor for Alpha-1D adrenoreceptor), for example.

Examples of proteins associated with Immunodeficiency may include A2M [alpha-2-macroglobulin]; AANAT [arylalkylamine N-acetyltransferase]; ABCA1 [ATP-binding cassette, sub-family A (ABC1), member 1]; ABCA2 [ATP-binding cassette, sub-family A (ABC1), member 2]; or ABCA3 [ATP-binding cassette, sub-family A (ABC1), member 3]; for example.

Examples of proteins associated with Trinucleotide Repeat Disorders include AR (androgen receptor), FMR1 (fragile X mental retardation 1), HTT (huntingtin), or DMPK (dystrophia myotonica-protein kinase), FXN (frataxin), ATXN2 (ataxin 2), for example.

Examples of proteins associated with Neurotransmission Disorders include SST (somatostatin), NOS1 (nitric oxide synthase 1 (neuronal)), ADRA2A (adrenergic, alpha-2A-, receptor), ADRA2C (adrenergic, alpha-2C-, receptor), TACR1 (tachykinin receptor 1), or HTR2c (5-hydroxytryptamine (serotonin) receptor 2C), for example.

Examples of neurodevelopmental-associated sequences include A2BP1 [ataxin 2-binding protein 1], AADAT [aminoadipate aminotransferase], AANAT [arylalkylamine N-acetyltransferase], ABAT [4-aminobutyrate aminotransferase], ABCA1 [ATP-binding cassette, sub-family A (ABC1), member 1], or ABCA13 [ATP-binding cassette, sub-family A (ABC1), member 13], for example.

In one aspect, the invention provides kits containing any one or more of the elements disclosed in the above methods and compositions. Elements may be provided individually or in combinations, and may be provided in any suitable container, such as a vial, a bottle, or a tube. In some embodiments, the kit includes instructions in one or more languages, for example in more than one language.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide. In some embodiments, the kit comprises one or more of the vectors and/or one or more of the polynucleotides described herein. The kit may advantageously allows to provide all elements of the systems of the invention.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Isolation/Purification of Exosomes, and RNA Extraction Therefrom (No Proteinase Treatment): Standard Exosome Isolation

The following protocol was used to isolate RNA from suspension cells such as K562 Cells. Buffers and some reagents refer to a mirVana RNA kit (Life technologies).

Day 1
Spin down about 72 million cells total in 6 50 mL Falcon tubes (12 million cells per tube) at 300×g for 5 minutes.
Aspirate media and resuspend each cell pellet in 43 mL exosome-free media. Transfer contents of each Falcon tube to T75 flask.

Day 2
After 24 hours, take off all media and divide among 50 mL falcon tubes. Spin at 300×g for 10 minutes at 4 degrees.
Transfer supernatant to new 50 mL tubes leaving cell pellet behind. Spin at 2000×g for 10 minutes at 4 degrees. Transfer supernatant to new 50 mL tubes leaving cell pellet behind.
Spin supernatant at 16,500×g for 20 minutes at 4 degrees.
Transfer supernatant to new 50 mL tubes, leaving pellet behind.
Pass supernatant through Steriflip 0.22 micron filter.
Transfer supernatant to pollyallomer ultracentrifuge tubes. Centrifuge at 120,000×g (26,500 RPM with SW32Ti rotor) for 70 minutes at 4 degrees.
Remove supernatant, leaving ˜2 cm of media above pellet. Add 5 mL PBS to each tube. Vortex on medium speed for a few seconds. Fill to top of each tube with PBS.
Again, centrifuge at 120,000×g for 70 minutes at 4 degrees.
Aspirate all of supernatant with Pasteur pipet without touching bottom of tube (where pellet is located).
Add 2 μL Superasin to each tube (SUPERase• In™ KNase Inhibitor from Life technologies).
Add 200 μL of Lysis/Binding Solution directly to the bottom of each ultracentrifuge tube. Pipet up and down. Transfer the contents of 3 ultracentrifuge tubes to one 1.5 mL Eppendorf tube.
Vortex briefly and place on ice.
Add 60 μL of miRNA Homogenate Additive to each tube ( 1/10 volume of lysate).
Vortex each tube and place on ice for 10 minutes.
Add a 600 μL of Acid-Phenol:Chloroform to each tube (volume that is equal to lysate volume before addition of miRNA Homogenate Additive).
Vortex for 30 seconds to mix thoroughly.
Centrifuge at maximum speed for 5 minutes (all spins at room temperature).
While tubes are spinning, transfer some Elution Solution to new 1.5 mL tube and pre-heat Elution Solution in heating block to 95° C. Also, put filter cartridges into collection tubes.
Carefully remove the upper (aqueous) phase and transfer to a new 1.5 mL tube.
Add 1.25 volumes of 100% ethanol to the transferred aqueous phase.
Pipet up and down and transfer up to 700 μL to a filter cartridge. Centrifuge at 10,000 RCF (10,000 RPM) for 15-30 seconds.
Discard flow-through and load the rest of the lysate/ethanol mixture. Centrifuge at 10,000 RCF (10,000 RPM) for 15-30 seconds.
Add 700 μL of miRNA Wash Solution 1 to filter and centrifuge at 10,000 RCF (10,000 RPM) for 15 seconds. Discard flow-through.
Add 500 μL of miRNA Wash Solution ⅔ to filter and centrifuge at 10,000 RCF (10,000 RPM) for 15 seconds. Discard flow-through.
Again, add 500 μL of miRNA Wash Solution ⅔ to filter and centrifuge at 10,000 RCF (10,000 RPM) for 15 seconds. Discard flow-through.
Put filter back in collection tube and spin for 1 minute at 10,000 RCF (10,000 RPM) to remove any residual ethanol from the filter.
Transfer filter cartridge with bound RNA to a new collection tube.
Add 100 μL of pre-heated Elution Buffer to the center of each filter. Centrifuge for 30 seconds at maximum speed to recover the RNA.
Store RNA at −80° C.

### Example 2

### Isolation/Purification of Exosomes, and RNA Extraction Therefrom (with Proteinase and RNase Treatment): Removal of Protein-RNA Complexes from the Exosome Pellet

The following protocol was used to isolate RNA from suspension cells such as K562 Cells. This protocol removes RNA-protein complexes from the exosomes. Buffers refer to a mirVana RNA kit (Life technologies).
Execute exosome isolation protocol (see example 1) on 6×12 million cells up to the end of first ultracentrifugal spin.
Take off complete supernatant of all six tubes. Resuspend each in 150 μL PBS. Label two tubes P1 and P2 and to these, add 5 uL of proteinase K (active conc. 500 μg/mL).
Incubate all tubes at 37° C. for 30 minutes.
Fill tubes with PBS and ultracentrifuge again.
After second spin, take off complete supernatant of all six tubes. Resuspend each in 150 μL PBS. Label the four unlabeled tubes NT1, NT2, PR1 and PR2.
Add 5 μL of proteinase K (active conc. 500 m/mL) to PR1 and PR2.
Incubate all tubes at 37° C. for 30 minutes.
Add 5 μL PMSF (from 20 mM stock; active conc. 1 mM) to PR1 and PR2.
Leave all tubes at RT for 10 minutes.
Add 0.5 μL RNase A/T1 (active conc. ˜3 μg/mL) to PR1 and PR2.
Incubate all tubes at 37° C. for 30 minutes.
Add 2 μL superasin to each tube. (SUPERase• In™ RN ase inhibitor from Life technologies).
Move contents of each tube to 1 Eppendorf tube (total volume should be ˜200 μL per tube due to residual liquid in UC tube), labeled accordingly, and proceed with mirVana RNA isolation using 300 μL lysis buffer.

### Example 3

### Chemical and Enzymatic Treatment of Exosomes

To achieve purified exosomes which are essentially free of extra-exosomal nucleic acid-protein complexes, the following procedure is provided. In sum, DNase is added during the preparation, then inactivated prior to lysing all of the vesicles which affords a composition which is essentially free of extra-exosomal nucleic acid-protein complexes. Briefly, exosome pellet—either at the wash step between ultracentrifugations or after the final ultracentrifugation, as indicated—was resuspended in 50-500 μL PBS or 0.5% Triton X-100 as indicated. For proteinase treatment, Proteinase K (Life Technologies) was added to a final concentration of 500 μg/mL, and samples were incubated at 37° C. for 30 minutes. Treatment was initially done in Proteinase K activity buffer (0.1 M NaCl, 10 mM Tris pH 8, 1 mM EDTA) rather than PBS, however reduced RNA yields from untreated exosomes resuspended in this buffer were observed; thus, all further treatments were performed in PBS. Proteinase was subsequently inactivated by the addition of phenylmethylsulfonyl fluoride (PMSF; Millipore) to 1 mM concentration. For RNase treatment, RNase Cocktail Enzyme Mix (Life Technologies) was added to a final concentration of 1.25 and 50 U/mL RNase A and T1, respectively, and samples incubated at 37° C. for 30 minutes. RNase was inactivated by the addition of SUPERasein (Life Technologies) to 20 U/mL concentration and the addition of ≥2 volumes lysis buffer from mirVana miRNA isolation kit (Life Technologies). For DNase treatment, Turbo DNase (Life Technologies) was added to a concentration of 26 U/mL, with Turbo DNase buffer added to 1× concentration where indicated, and samples incubated at 37° C. for 30 minutes. DNase was inactivated by the addition of EDTA to 15 mM followed by incubation at 75° C. for 10 minutes.

### Example 4

### CD81 and CD63 Exosome Isolation with Mouse IgG Beads (Pull Down Purification)

Day 0 (or Earlier)
1. Mix 50 mL FBS with 500 mL IMDM and 5 mL P/S. Filter through 0.22 μM filter. Grow cells.

Day 1
2. Spin down 72 million cells total in 6 50 mL Falcon tubes at 300×g for 5 minutes.
3. Aspirate media and resuspend each cell pellet in 43 mL AIM-V. Transfer contents of each Falcon tube to T75 flask.

Day 2
4. After 24 hours, take off all media and divide among 50 mL falcon tubes. Spin at 300×g for 10 minutes at RT.
5. Transfer supernatant to new 50 mL tubes leaving cell pellet behind. Spin at 2000×g for 10 minutes at RT. Transfer supernatant to new 50 mL tubes leaving cell pellet behind.
6. Spin supernatant at 16,500×g for 20 minutes at 4 degrees.
7. Transfer supernatant to new 50 mL tubes, leaving pellet behind.
8. Pass supernatant through Steriflip 0.22 micron filter.
9. Transfer supernatant to pollyallomer ultracentrifuge tubes. Centrifuge at 120,000×g (26,500 RPM with SW32Ti rotor) for 70 minutes at 4 degrees.
10. During this spin, make fresh Isolation Buffer (PBS supplemented with 1 mg/mL BSA, filtered through 0.22 μm filter) and prepare hot plate at 95° C.
11. Also during first ultracentrifuge spin, prepare beads:
   a. resuspend anti-mouse IgG Dynabeads by mixing for >10 min or vortexing gently for 30 s.
   b. transfer 100 μL (4×107) beads each into 3 different Biotix 2 mL tubes labelled C, 81 and 63.
   c. wash the magnetic beads by adding 1 mL of Isolation Buffer. Mix well.
   d. place tubes on the magnet for 2 minutes and remove supernatant carefully.
   e. remove tubes from magnet and add 100 μL isolation buffer.
   f. To 81, add 20 μL (4 μg) anti-human CD81 antibody, clone 1.3.3.22
   g. To 63, add 8 μL (4 μg) anti-human CD63 antibody, clone h5c6
h. To C, add 4 μL (4 μg) ctrl antibody (mouse mAb mCherry, 1C51)
i. Incubate on rotating rack in cold room until end of isolation (˜3 hours)
12. Remove supernatant, leaving ˜2 cm of media above pellet. Add 5 mL PBS to each tube. Vortex on medium speed for a few seconds. Fill to top of each tube with PBS.
13. Again, centrifuge at 120,000×g for 70 minutes at 4 degrees.
14. Aspirate all of supernatant with Pasteur pipet without touching bottom of tube (where pellet is located).
15. Add 80 μL PBS to each tube and let sit for ˜15 minutes.
16.Resuspend and pool all tubes into a biotix tube labelled P. Measure total volume, should be ˜600 μL due to 20 μL residual liquid after aspiration.
17. Retrieve bead tubes from cold room, spin briefly and place on magnet.
18. Do 2×900 μL washes in isolation buffer to remove excess antibody.
19. Split pooled pellets ⅙ into each of the biotix tubes. Add isolation buffer to each bead tube to 200 μL total volume. Put all on rotating rack in cold room overnight (16 hours)
20. Add 33 μL 4× SB (133 μL total volume) to remaining 100 μL of exosomes in P and boil at 95° for 5 min. Place immediately on ice and freeze at −80°

Day 3
21. After 16 h, centrifuge all tubes from cold room briefly to collect samples.
22. Place C, 81 and 63. on magnet for two minutes. Collect supernatants and store in new tubes labelled C-FT, 81-FT, 63-FT respectively.
23. Wash beads in each tube with 500 μL Isolation Buffer. Leave 2 min on magnet before collecting wash supernatants. Add each wash to respective FT tube. Store at 4° C.
24. Add 133 μL 1× Sample Buffer to C, 81 and 63. and boil at 95° for 5 min. Place immediately on ice for 5 min, then place on magnet for 2 min, collect supernatants and freeze at −80° in new tubes.
25. Assemble all flow-through tubes and add each to its own ultracentrifuge tube. Fill tubes with PBS and spin 180 minutes at 120 000 g.
26. After ultracentrifugation, remove supernatant entirely, add 80 μL PBS and leave pellets for ˜15 minutes.
27. Resuspend (should be about 100 μL) move to labelled biotix tubes and add 33 μL 4× Sample Buffer to each.
28. Boil at 95° C. for 5 min. Place immediately on ice and freeze at −80° C.

### Example 5

### Analysis of RNA Contents of Exosomes as a Function of Exosome Purification Method—Size Distribution

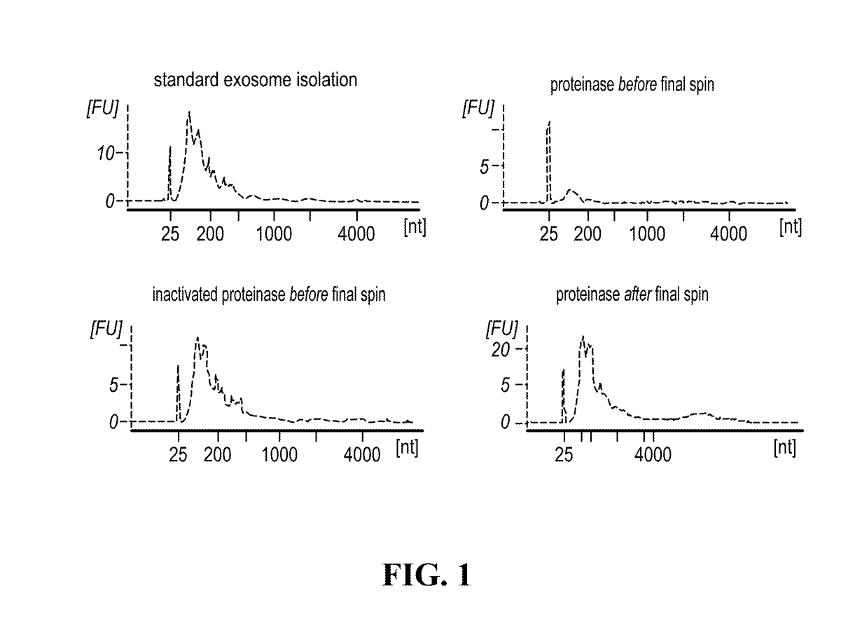

FIG. 1 shows graph of RNA fluorescence unit (FU) plotted against RNA size (nt), wherein “final spin” refers to the final centrifugation step.

The results allow comparison and validation of corresponding purification methods.

### Example 6

### Analysis of RNA Contents of Exosomes as a Function of Exosome Purification Method—Electron Microscopy Imaging

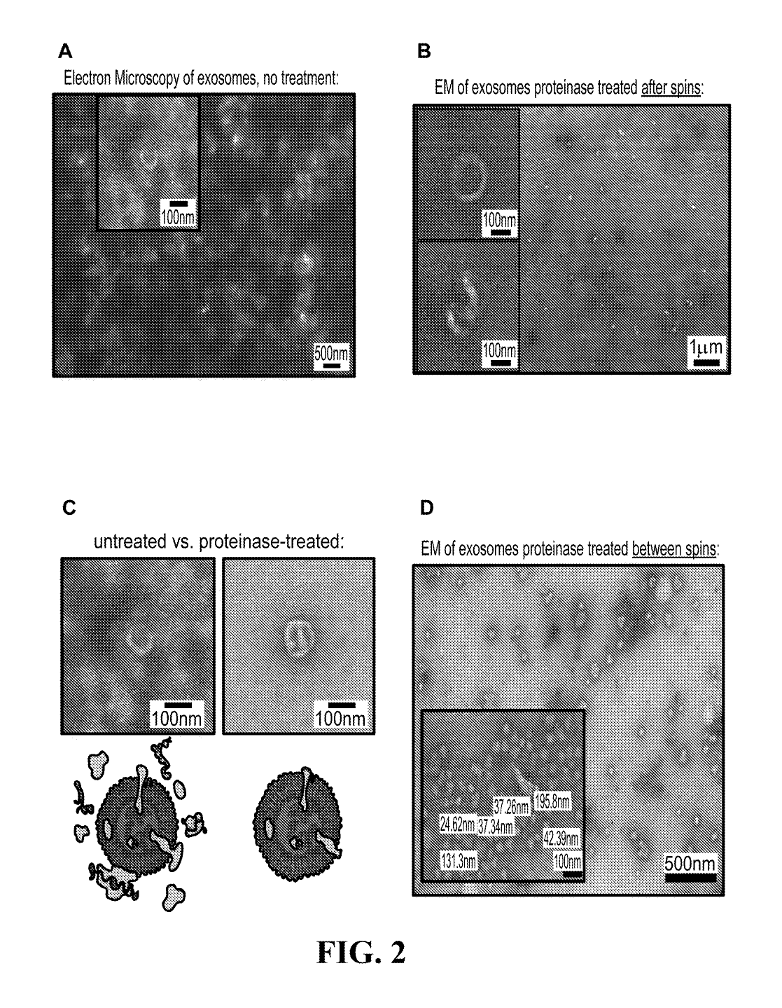

FIGS. 2A-D show electron microscopy (EM) photographs of exosome preparations, wherein “no treatment” refers to a protocol according to example 1; “after spins” refers to a protocol according to example 2; “between spins” denotes a protocol according to example 1, except that additional proteinase treatment occurred between the two ultracentrifugation steps.

The results show that the method used for exosome preparation affects exosome integrity. EM data allow comparison and validation of exosome purification methods.

Vesicles Electron Microscopy Prep

Stain prep
Weigh 60 mg powdered Uranyl Formate into clean 10 mL beaker with stir stick in radioactivity hood.
Move this to the stir plate (make sure stirring is OFF) and cover with the big beaker with tin foil.
Fill another clean 10 mL beaker with 3 mL water and heat this up (not on the same hot plate) until it's super boiling/as hot as possible. Ensure not to lose too much water to evaporation.
Quickly pour this into other beaker with powder and start stirring. Stir vigorously for 2 minutes protected by tin foil.
Using BD 5 mL syringe (with black lining inside, not the normject ones) suck up stain and then using corning 0.45 μm filter to filter it, deposit into 15 mL falcon tube. Label and wrap in tin foil.
Wipe beaker with Kim wipe. throw this, gloves, syringe and filter into radioactive waste
   Sample prep
Good sample concentration is in the range of 1 nM
Use special tweezers to put grids on parafilm-covered slide, dark shiny side up.
Put slide in glow discharger, close lid carefully, hit start.
Pick up grids with tweezers at the edge, don't pinch too hard. Put tweezers down (still holding grid) and pipet 3.5 μL of sample onto it. Leave 1 minute. This time changes depending on salt concentration etc.
Wick away liquid with a piece of filter paper
Add 3.5 μL stain, leave 30 seconds, then wick away this as well.
Find a holder and carefully put grids down with dark side up, use this to carry to EM room

### Example 7

### Analysis of RNA Contents of Exosomes as a Function of Exosome Purification Method—qRT-PCR Analysis—Validation of the Purification Method

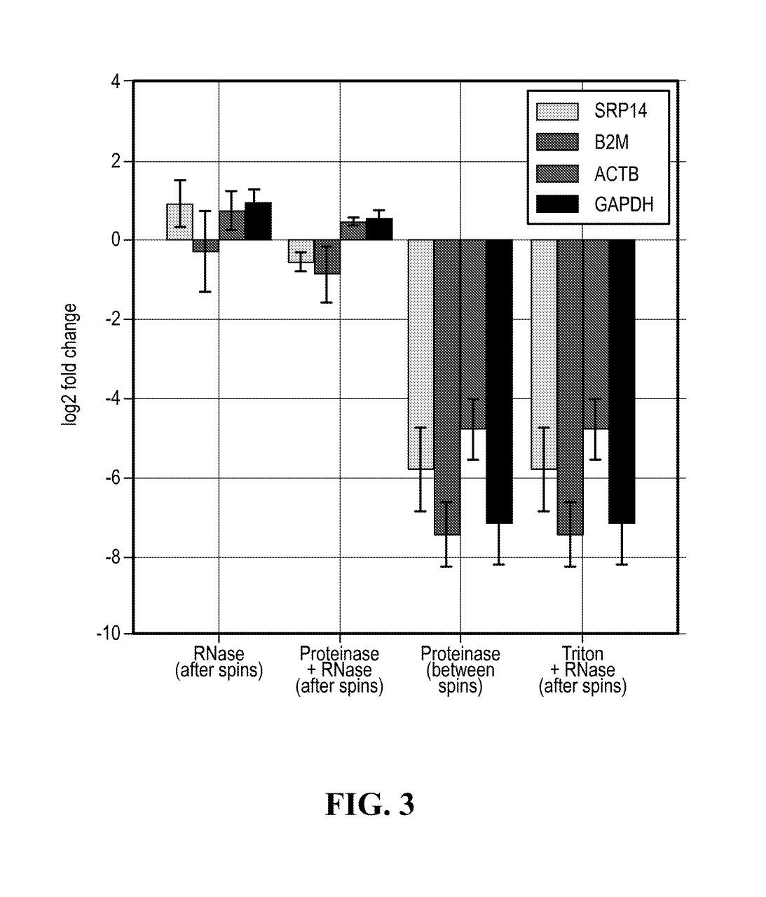

FIG. 3 shows qRT-PCR data of exosome RNA for 4 mRNAs that were previously found in exosome RNA-Seq data.

The qRT-PCR is performed for various conditions of exosome purification methods. All runs are normalized to RNA from the ‘regular’ exosome isolation (Example 1). The conditions for exosome purification are as follows:

(1) RNase treatment only
(which is expected not be sufficient if RNA is also protected by proteins as was shown for extracellular microRNAs in Arroyo et al, Proc Natl Acad Sci USA. 2011 Mar. 22; 108(12):5003-8. doi: 10.1073/pnas.1019055108. Epub 2011 Mar. 7.2011; Turchinovich et al Nucleic Acids Res. 2011 Sep. 1; 39(16):7223-33. doi: 10.1093/nar/gkr254. Epub 2011 May 24.)

(2) proteinase+RNase treatments after spins
(protocol as per Example 2; also see below)

(3) proteinase treatment (between spins)
This is the method described in previous publications such as Valadi et al, Nat Cell Biol. 2007 June; 9(6):654-9. Epub 2007 May 7. As shown by EM (see above and FIG. 2d), this method compromises exosome integrity.
In accordance with the EM data, the qRT-PCR results show a decrease in mRNA levels.

(4) Triton+RNase treatments
This is a control run, wherein where Triton treatment is used to break open the vesicles, and samples are further treated with RNase. The results show drastic reduction in levels of mRNA.

R/T Isolation for qPCR
1. Execute exosome isolation protocol on 6×12 million cells up to the end of second ultracentrifugal spin.
2. Take off complete supernatant of all six tubes.
3. Resuspend 4 pellets in 150 μL PBS. Label them NT1, NT2, R1 and R2.
4. Resuspend other 2 pellets in 3% Triton. Label them TR1 and TR2,
5. Add 0.5 μL RNase A/T1 (active conc. ˜3 μg/mL) to R1, R2, TR1 and TR2,
6. Incubate all tubes at 37° C. for 30 minutes.
7. Add 2 μL superasin to each tube (SUPERase• In™ RNase inhibitor from Life technologies).
8. Proceed with mirVana RNA isolation using 300 μL lysis buffer (mirVana RNA kit from Life technologies).

P/R isolation for qPCR
1. Execute exosome isolation protocol on 6×12 million cells up to the end of first ultracentrifugal spin.
2. Take off complete supernatant of all six tubes. Resuspend each in 150 μL PBS. Label two tubes P1 and P2 and to these, add 5 μL of proteinase K (active conc. 500 μg/mL).
3. Incubate all tubes at 37° C. for 30 minutes.
4. Fill tubes with PBS and ultracentrifuge again.
5. After second spin, take off complete supernatant of all six tubes. Resuspend each in 150 μL PBS. Label the four unlabeled tubes NT1, NT2, PR1 and PR2.
6. Add 5 uL of proteinase K (active conc. 500 μg/mL) to PR1 and PR2.
7. Incubate all tubes at 37° C. for 30 minutes.
8. Add 5 μL PMSF (from 20 mM stock; active conc. 1 mM) to PR1 and PR2.
9. Leave all tubes at RT for 10 minutes.
10. Add 0.5 μL RNase A/T1 (active conc. ˜3 μg/mL) to PR1 and PR2.
11. Incubate all tubes at 37° C. for 30 minutes.
12. Add 2 μL superasin to each tube (SUPERase• In™ RNase inhibitor from Life technologies).
13. Proceed with mirVana RNA isolation using 300 μL lysis buffer (mirVana RNA kit from Life technologies).

qRT-PCR
1. After collecting cell and exosome RNA, dilute 100 ng cell RNA to 100 μL with H₂O. Add 2 μL Turbo DNase (Lifetech), 2 μL Superasin, and 10 μL DNase buffer to each sample.
2. Incubate at 37° C. for 30 minutes.
3. Clean and concentrate using Zymo RNA Clean and Conncentrate kit according to instructions and elute in 16 μL H₂O.
4. Perform reverse transcription using Superscript VILO cDNA Synthesis kit with 14 μL of RNA in a 20 μL reaction.
5. Perform qPCR using KAPA Fast qPCR SYBR mix (KAPA Biosystems) with 2 μL of cDNA per reaction.

The following primers were used:

SRP14-F
GGGTACTGTGGAGGGCTTTG
SRP14-R
AGGAGGTTTGAATAAGCCATCTGA
B2M-F
GTATGCCTGCCGTGTGAAC
B2M-R
AAAGCAAGCAAGCAGAATTTGG
ACTB-F
CGGCATCGTCACCAACTG
ACTB-R
AACATGATCTGGGTCATCTTCTC
GAPDH-F
GGTGGTCTCCTCTGACTTCAACA
GAPDH-R
GTTGCTGTAGCCAAATTCGTTGT

### Example 8

### Correlation of Exosomal RNA Content with Cellular RNA Content

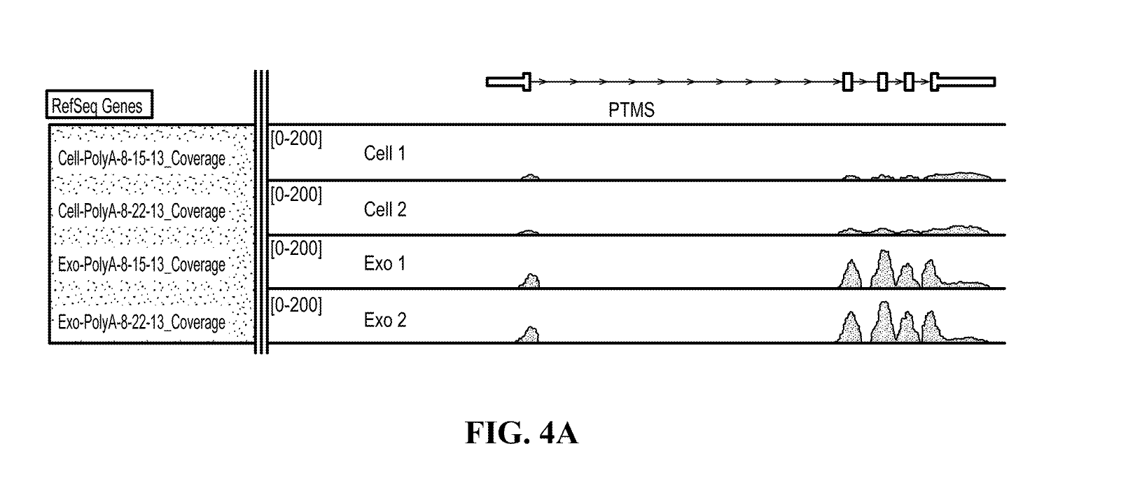

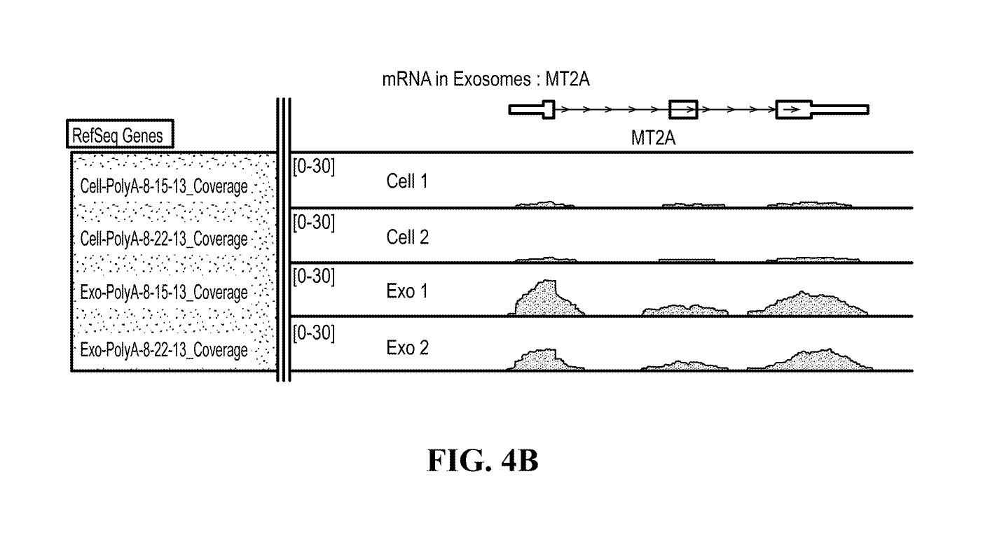

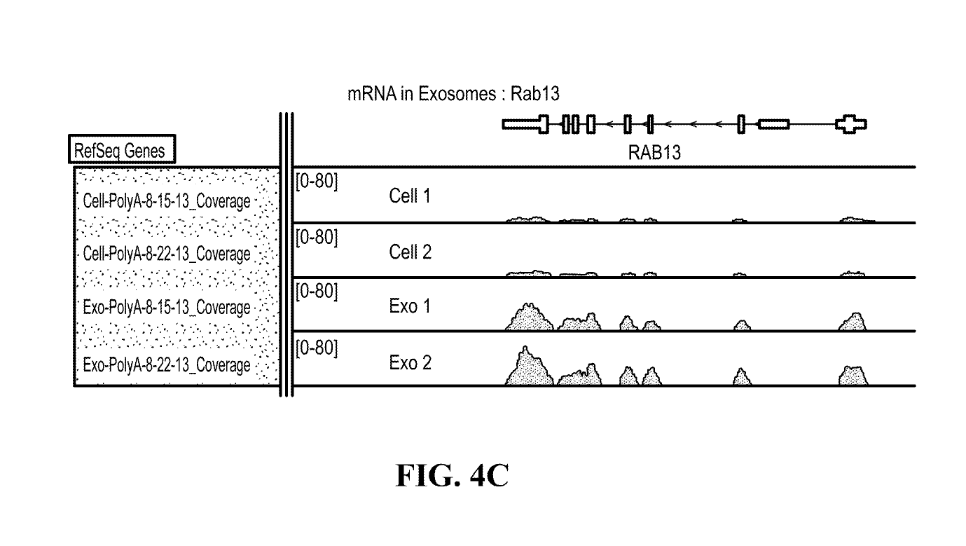

FIGS. 4A-C show RNA-Seq data, showing that the RNA profile of mRNAs in exosomes reflects that of the donor cells. This indicates that the exosomes provide an accurate snapshot of the transcriptome of the cells they come from. Exosome preparation was according to the standard exosome isolation procedure (as in Example 1, without proteinase/RNase).

RNA-Seq
1. After collecting cell and exosome RNA, dilute 100 ng cell RNA to 100 μL with H₂O. Add 2 μL Turbo DNase (Lifetech), 2 μL Superasin, and 10 μL DNase buffer to each sample.
2. Incubate at 37° C. for 30 minutes.
3. Clean and concentrate using Zymo RNA Clean and Conncentrate kit according to instructions and elute in 10 μL H₂O.
4. Perform a PolyA Selection using Dynabeads mRNA Purification kit (Lifetech).
5. Proceed with RNA-Seq library prep protocol as described in: Perturbation of m6A writers reveals two distinct classes of mRNA methylation at internal and 5′ sites. Schwartz S, Mumbach M R, Jovanovic M, Wang T, Maciag K, Bushkin G G, Mertins P, Ter-Ovanesyan D, Habib N, Cacchiarelli D, Sanjana N E, Freinkman E, Pacold M E, Satija R, Mikkelsen T S, Hacohen N, Zhang F, Carr S A, Lander E S, Regev A. Cell Rep. 2014 Jul. 10; 8(1):284-96. doi: 10.1016/j.celrep.2014.05.048. Epub 2014 Jun. 26.

### Example 9

### Exosome-Mediated RNA Transfer Experiment Between HEK293 and K562 Cells

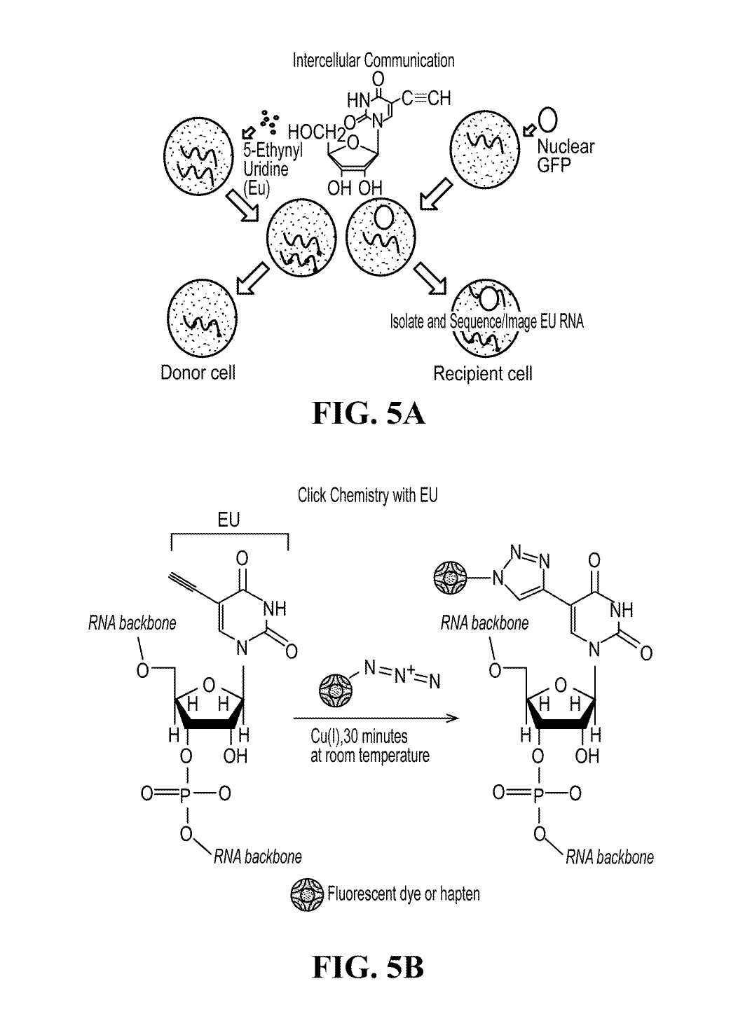

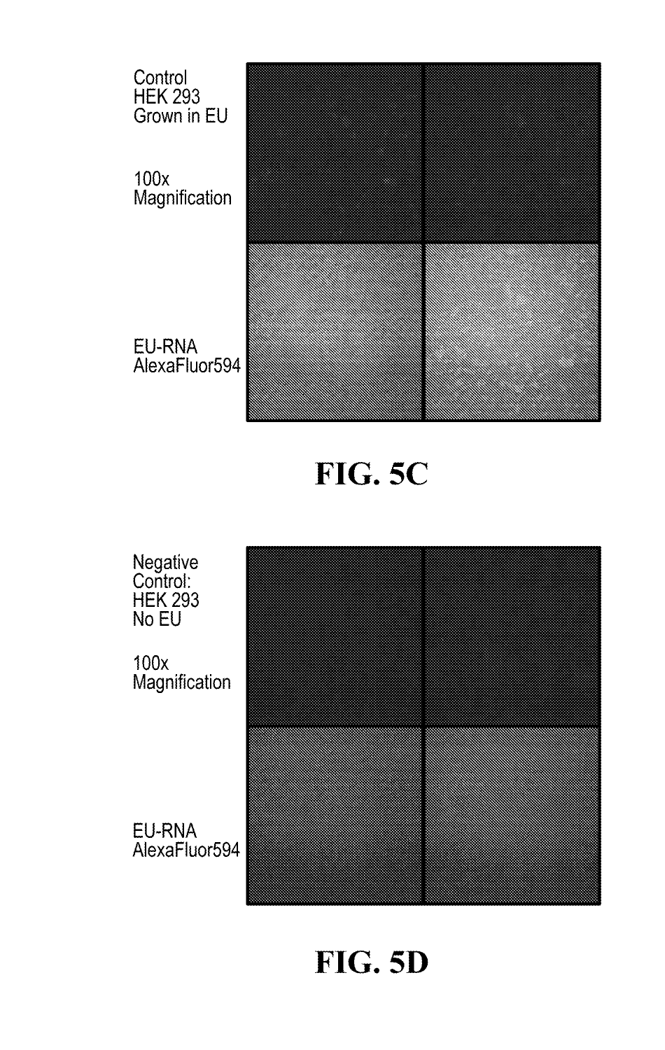

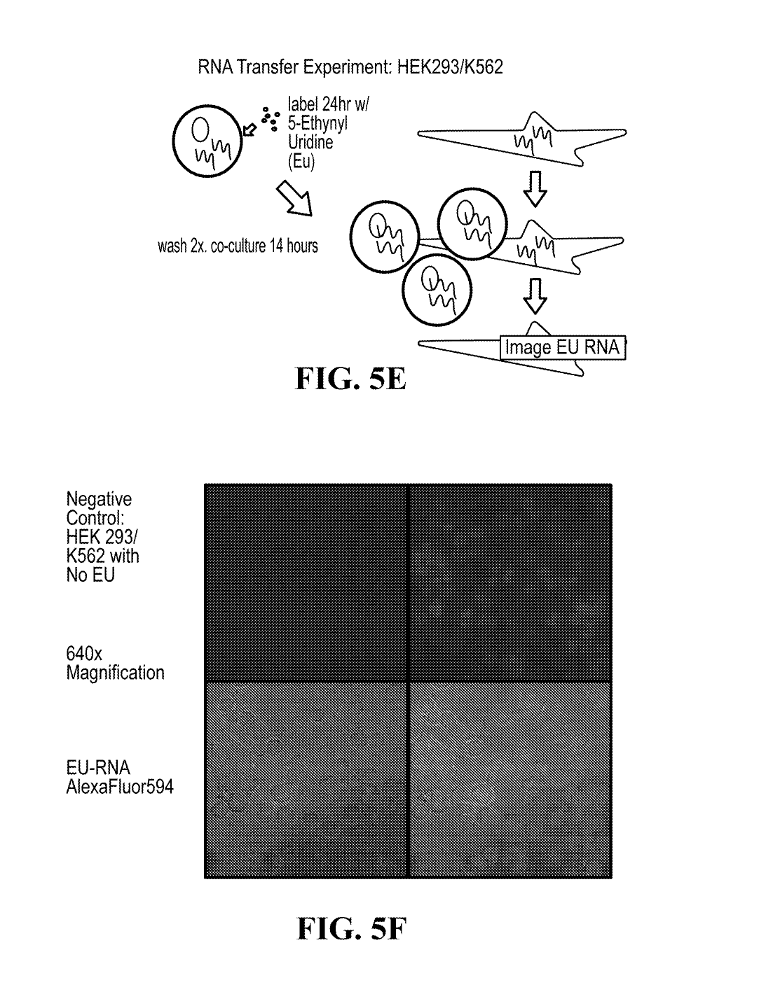

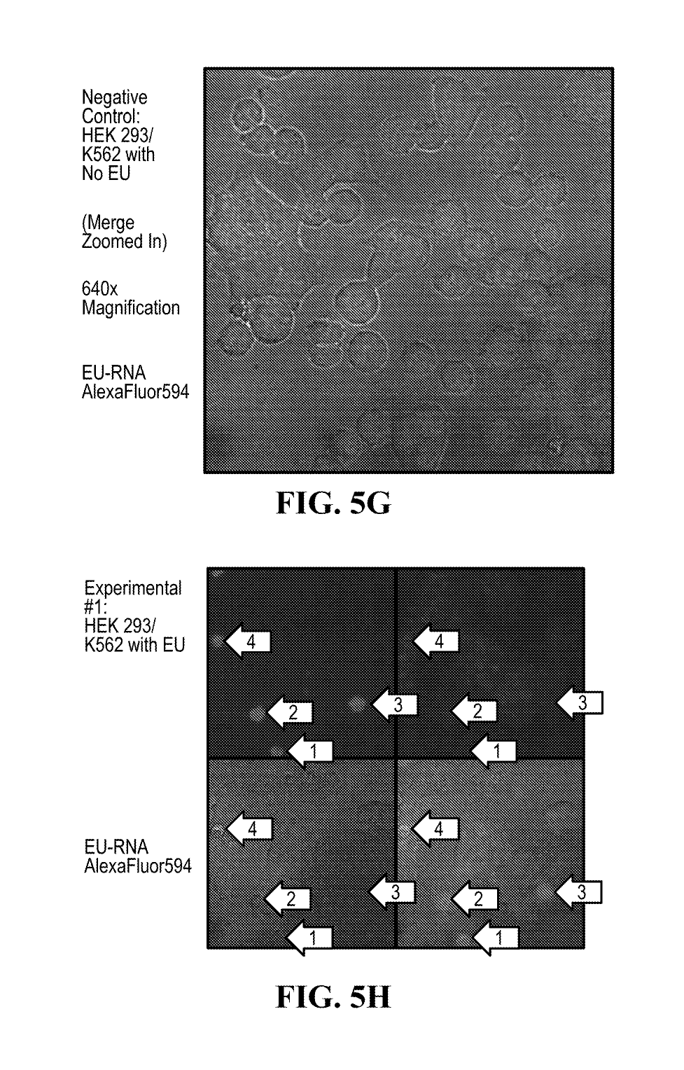

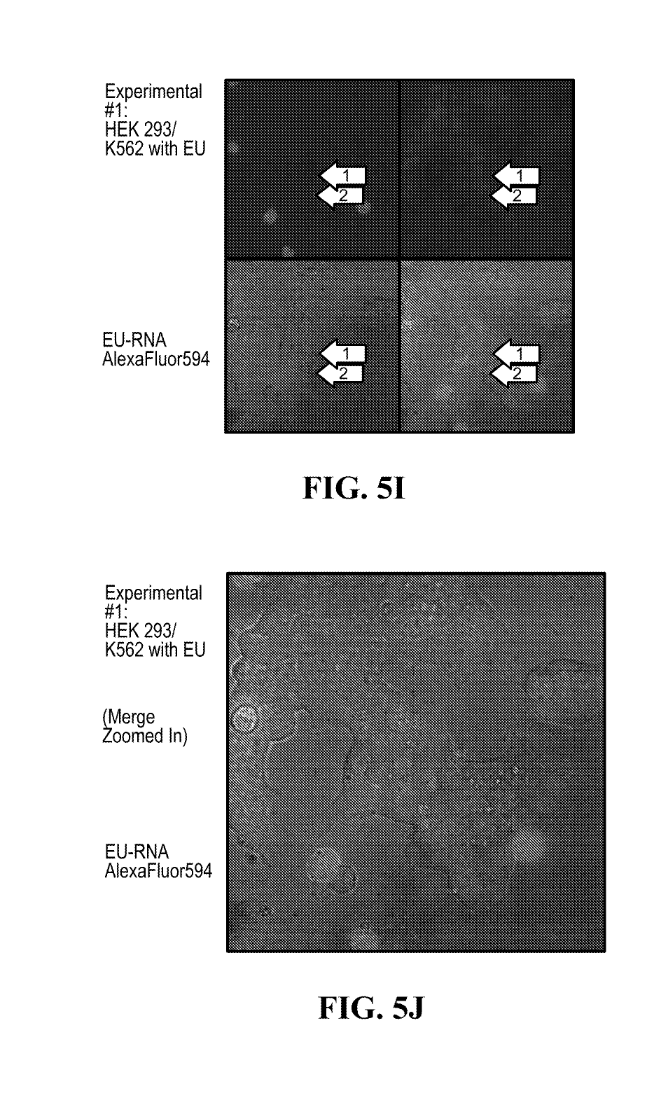

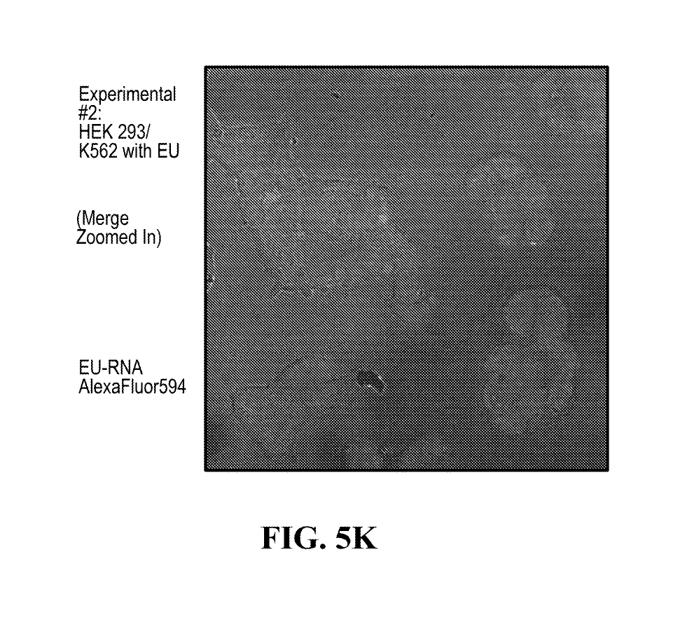

FIGS. 5A-K show fluorescence imaging of cells using EUclick chemistry.

This example shows results from a system that allows detection of potential endogenous RNA transfer between cells in a co-culture system by feeding donor cells with a modified nucleotide (5-ethynyl uridine, EU) that gets incorporated into its RNA and then co-culturing donor cells with unlabeled acceptor cells.

Click Chemistry is then used to detect RNA with the modified nucleotides by conjugate of a fluorophore to the EU. These results suggest the presence of RNA transfer. The white arrows point to spots of transferred RNA in the HEK293 acceptor cells. The green arrows just show the donor K562 cells.

EU-RNA Transfer Experiments
K562 and HEK293 cells were both obtained from ATCC.
K562 cells were incubated with 5-ethynyl uridine (Lifetech) diluted to 2 mM for 24 hours.
K562 and HEK 293 cells were co-cultured for 24 hours.
Cells were imaged using Click-IT RNA Alexa Fluor 594 Imaging kit (Lifetech)

### Example 10

### Exosome-Mediated RNA Transfer Experiment Between Co-Cultured Cell Lines

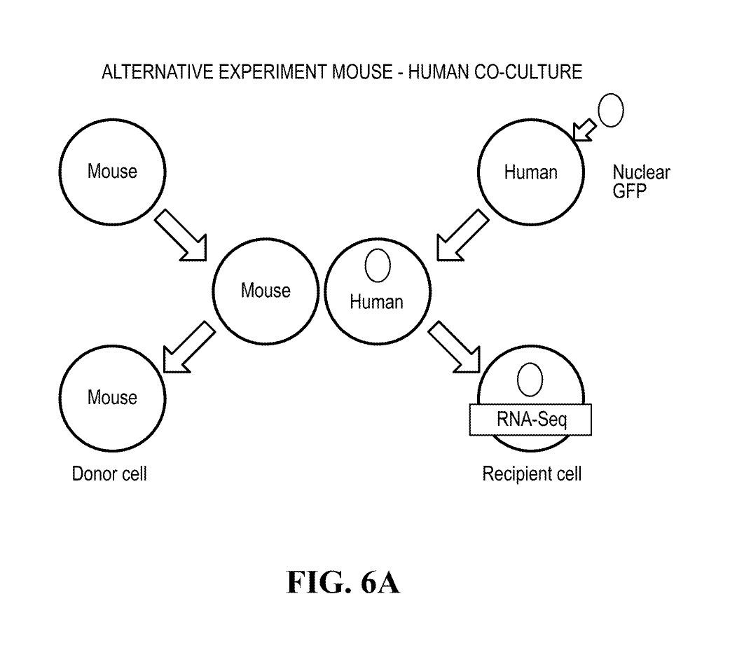

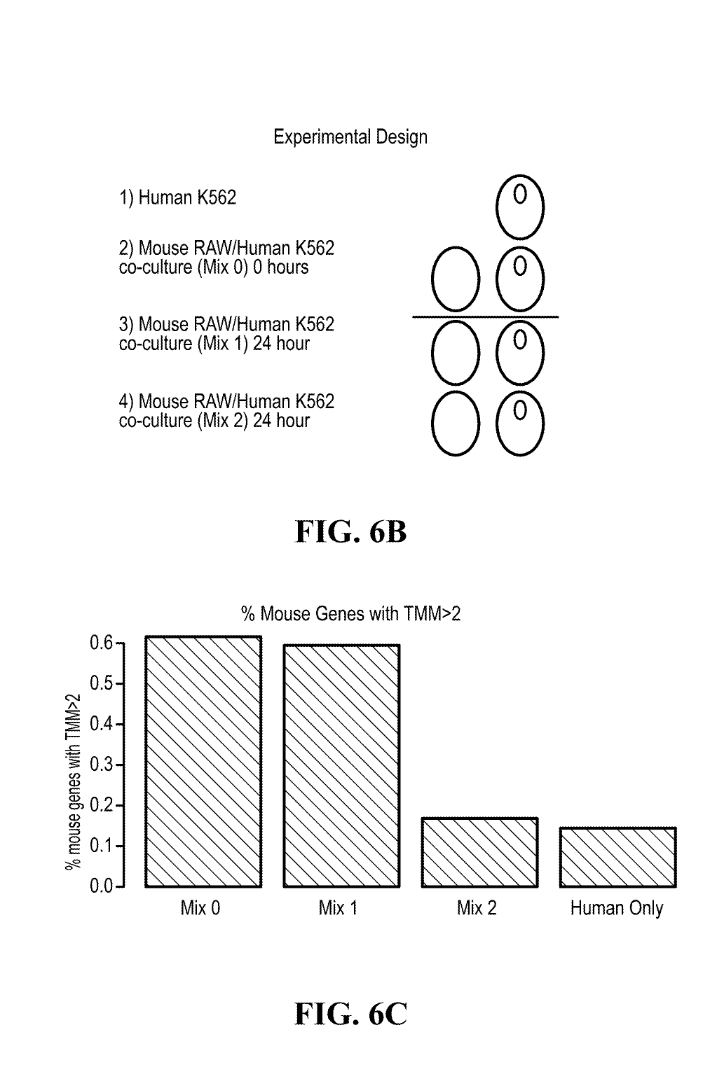

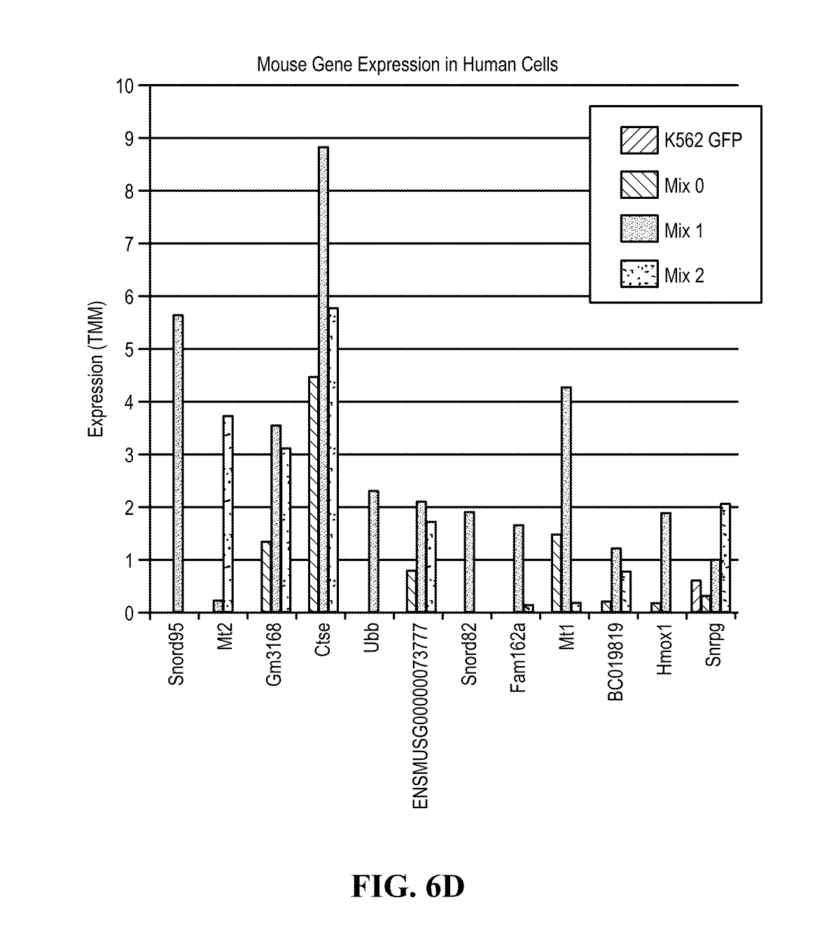

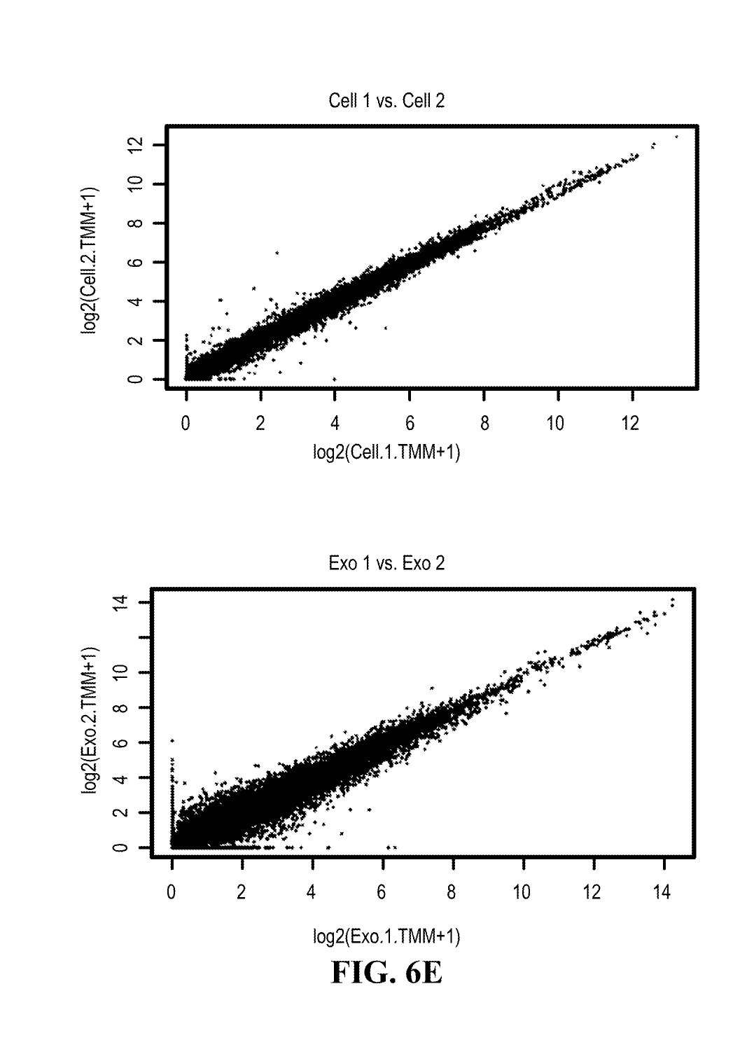

FIGS. 6A-D show principle and results of an experiment to assess possible exosome mediated RNA transfer between co-cultured cell lines.

This example illustrates a way to detect potential RNA transfer using unlabeled RNA. The principle is to co-culture mouse and human cells, separate them back out and use regular RNA-Seq to detect mouse transcripts in human cells. This technique relies on a principle similar to that of Example 7, but without using labeled nucleotides. Using this method, it was possible to detect some RNAs transferred but the strongest signal came from two mouse endogenous retrovirus RNAs (labeled as Gm3168 and Ctse).

Mouse Human RNA Transfer Experiments
Human K562 and Mouse RAW Macrophage cells were both obtained from ATCC.
K562 cells were infected with virus expressing GFP.
K562 cells were FACS sorted to all be GFP positive.
K562 GFP cells were co-cultured with Mouse RAW cells for 24 hours or 0 hours (as a control).
K562 GFP cells were FACS sorted for GFP positive cells to separate from Mouse cells after 24 hour co-culture (2 biological replicates: Mix 1 and Mix 2). The 0 hour co-culture was also sorted, as well as a control of just K562 cells that never interacted with mouse cells.
RNA was extracted using MirVana kit (Lifetech),
200 ng cell RNA to 100 μL with H2O. Add 2 μL Turbo DNase (Lifetech), 2 μL Superasin (Life technologies), and 10 μL DNase buffer to each sample.
Incubation at 37° C. for 30 minutes.
Clean and concentrate using Zymo RNA Clean and Concentrate kit according to instructions and elute in 10 μL H₂O.
PolyA Selection using Dynabeads mRNA Purification kit (Lifetech).
Proceed with RNA-Seq library prep protocol as described in: Perturbation of m6A writers reveals two distinct classes of mRNA methylation at internal and 5′ sites. Schwartz S, Mumbach M R, Jovanovic M, Wang T, Maciag K, Bushkin G G, Mertins P, Ter-Ovanesyan D, Habib N, Cacchiarelli D, Sanjana N E, Freinkman E, Pacold M E, Satija R, Mikkelsen T S, Hacohen N, Zhang F, Carr S A, Lander E S, Regev A. Cell Rep. 2014 Jul. 10; 8(1):284-96. doi: 10.1016/j.celrep.2014.05.048. Epub 2014 Jun. 26.

### Example 11

### Exosome Key Results

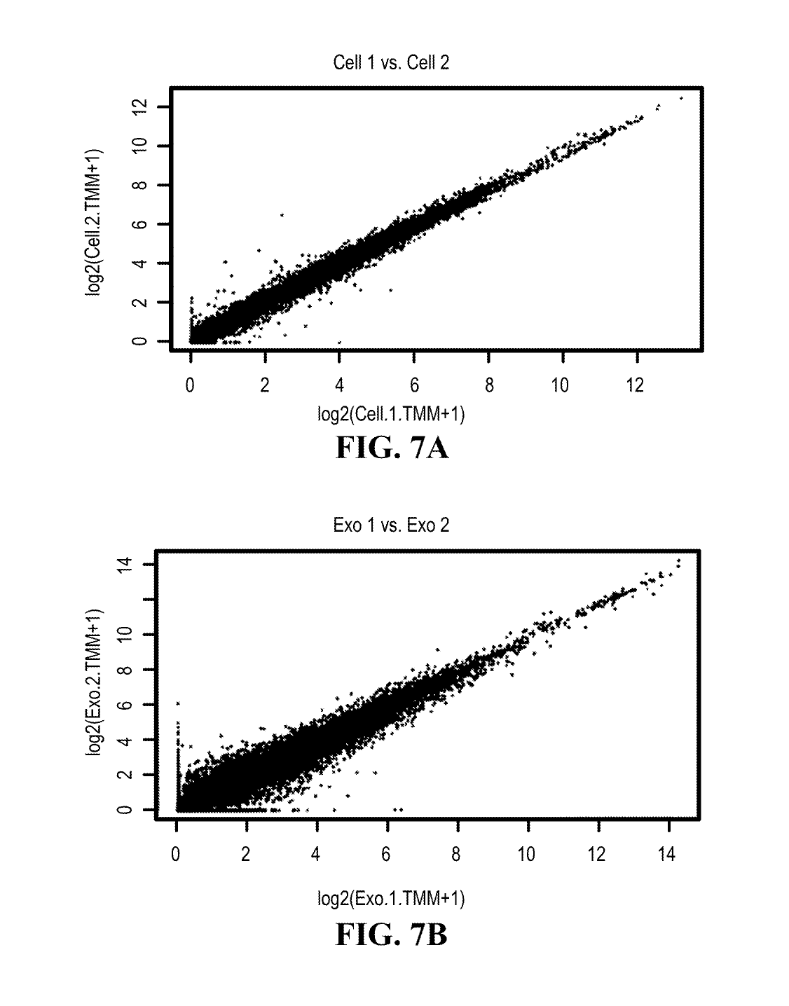

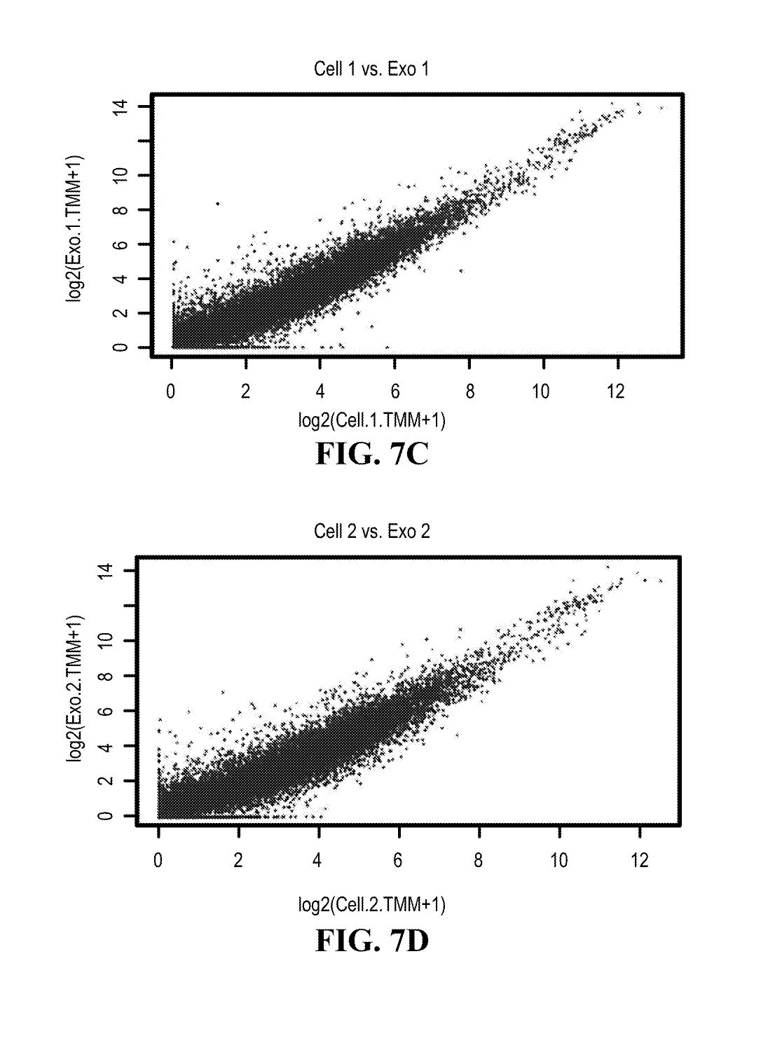

FIGS. 7A-D show poly A selected mRNA from two replicates of K562 cells and their exosomes was compared using RNA-Seq. The bottom two panels show that cell and exosome mRNA is correlated in expression for protein-coding genes.

Applicants have sequenced the mRNA of exosomes from K562 cells and compared the RNA profile of the donor cells to that of the exosomes. Applicants have found that the mRNA profiles of exosomes reflects the trasnscriptome of the donor cells. Thus, using exosomes as a non-invasive read-out of the transcriptome of inaccessible cell types is possible.

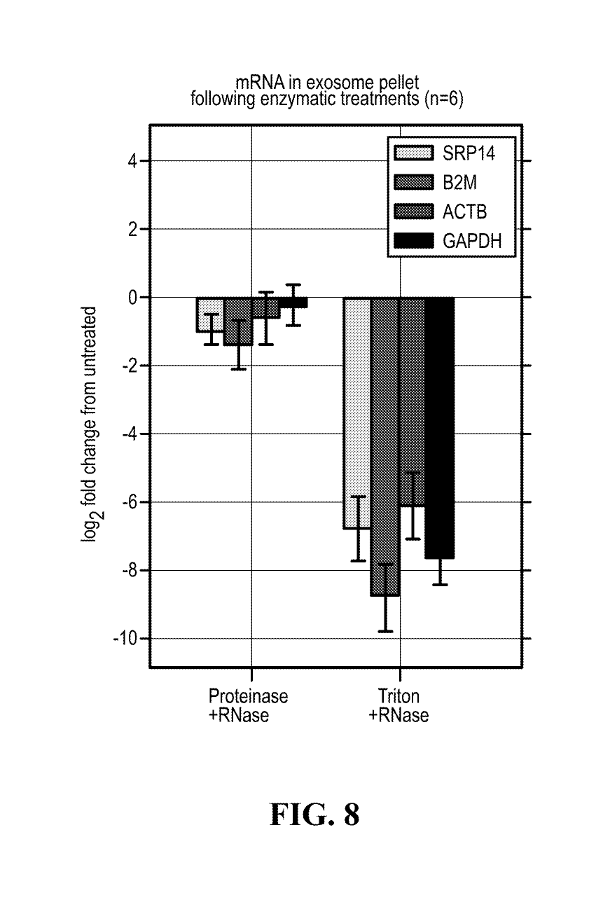

FIG. 8 illustrates mRNA in exosome pellet following enzymatic treatments. RNA from untreated exosomes and proteinase/RNase treated exosomes was compared using qRT-PCR for four mRNAs. There was very little or no change, indicating that the RNA is inside. As a control, vesicles with the detergent Triton were lysed and then treated with RNase.

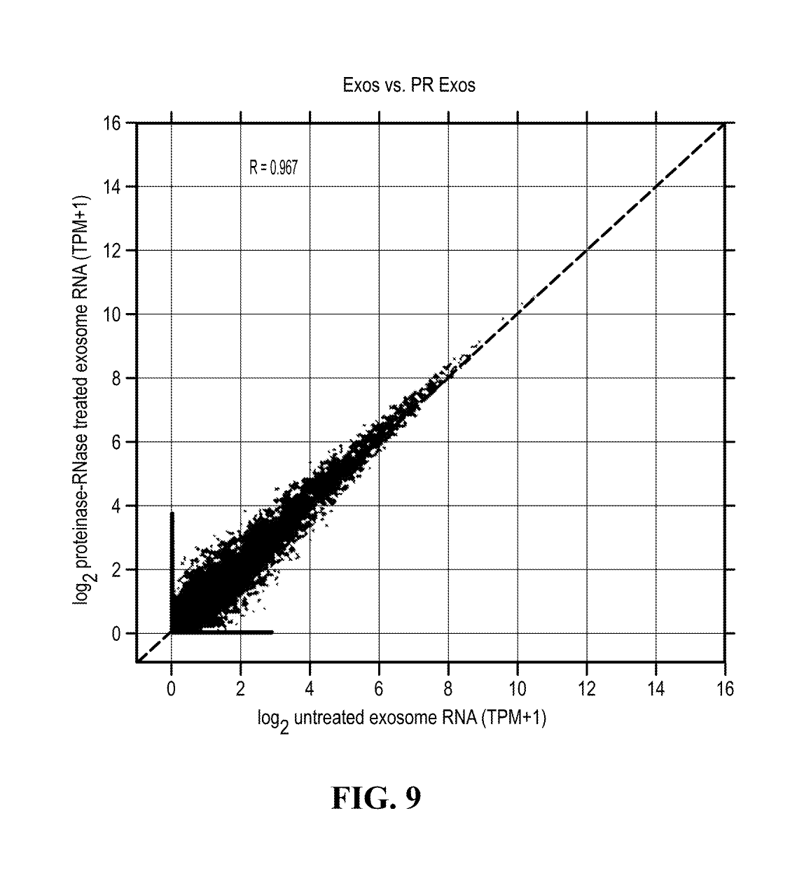

FIG. 9 illustrates Poly A enriched mRNA from untreated exosomes and proteinase/RNAse treated exosomes was compared using RNA-Seq. The mRNA is strongly correlated, indicating that the mRNA isolated via ultracentrifugation in the exosome pellet is inside the vesicles.

Applicants have confirmed that the mRNA in the exosome isolated product is really inside exosomes after developing a protocol to degrade all RNAs not in vesicles by enzymatic treatment with proteinase and then RNAse. Applicants find a very high correlation between the mRNA profiles in the untreated exosome pellet and the proteinase/RNAse treated pellet, indicating the sequenced mRNA is really inside the vesicles. Applicants have confirmed these results through qRT-PCR as well.

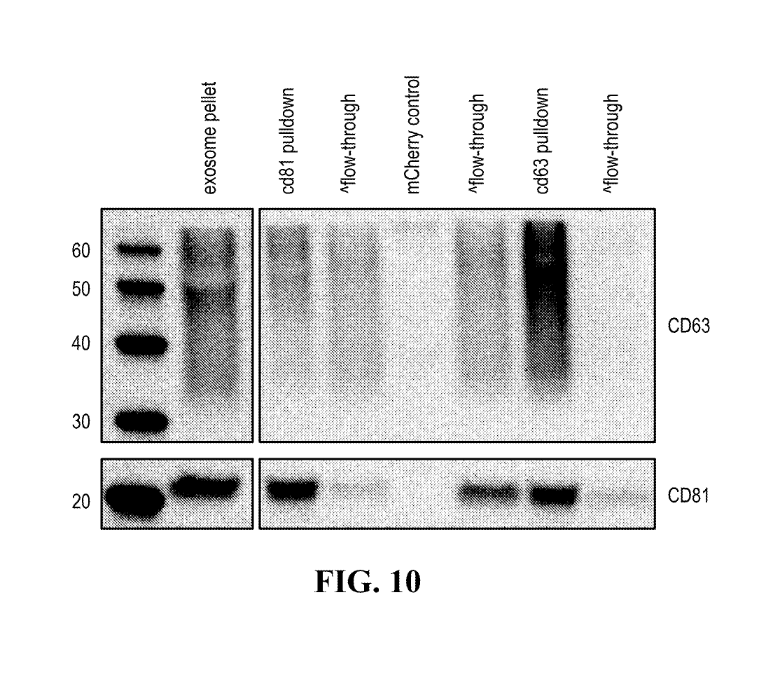

FIG. 10 illustrates targeted pull down exosome subpopulations based on their protein marker using antibody conjugated magnetic beads. CD63 is a glycosylated protein between 30 and 60 kDa. CD81 shows up as a distinct band between 20 and 30 kDa. mCherry is used as a non-specific control. This protocol/technique was developed to isolate specific exosome subpopulations by specific membrane proteins using antibody-conjugated magnetic beads. Further, the technique has been validated in K562 exosomes using the canonical exosome markers CD63 and CD81.

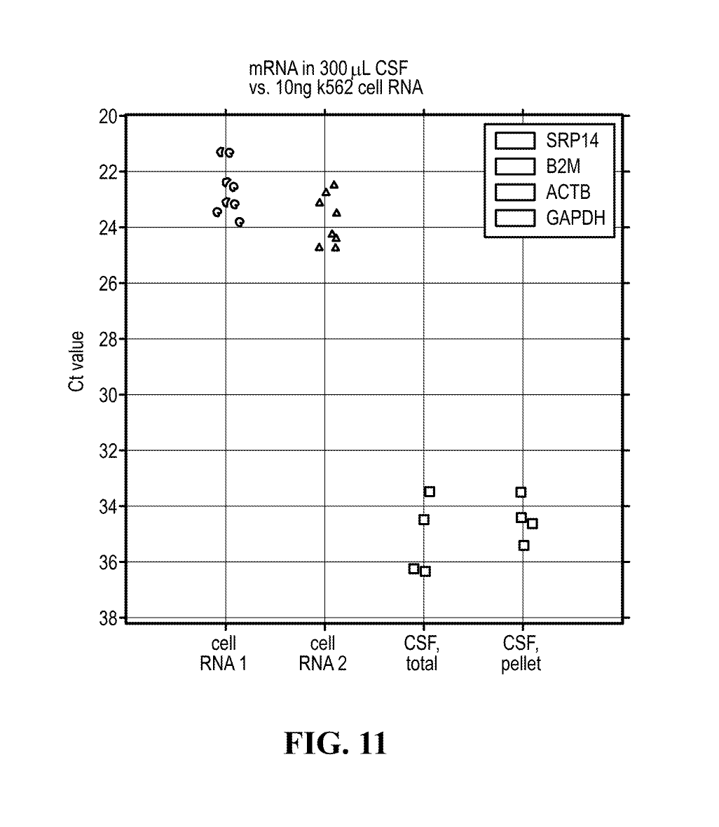

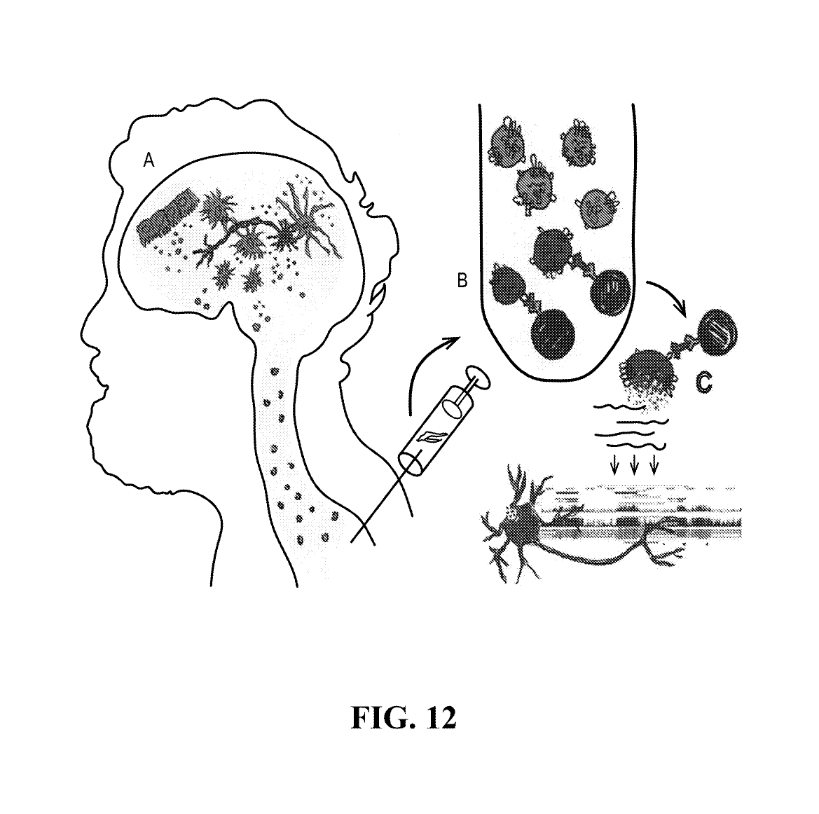

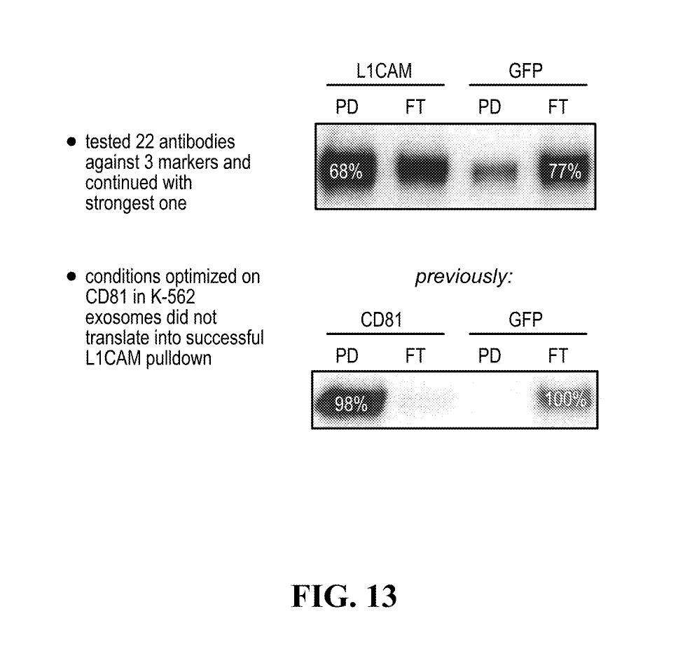

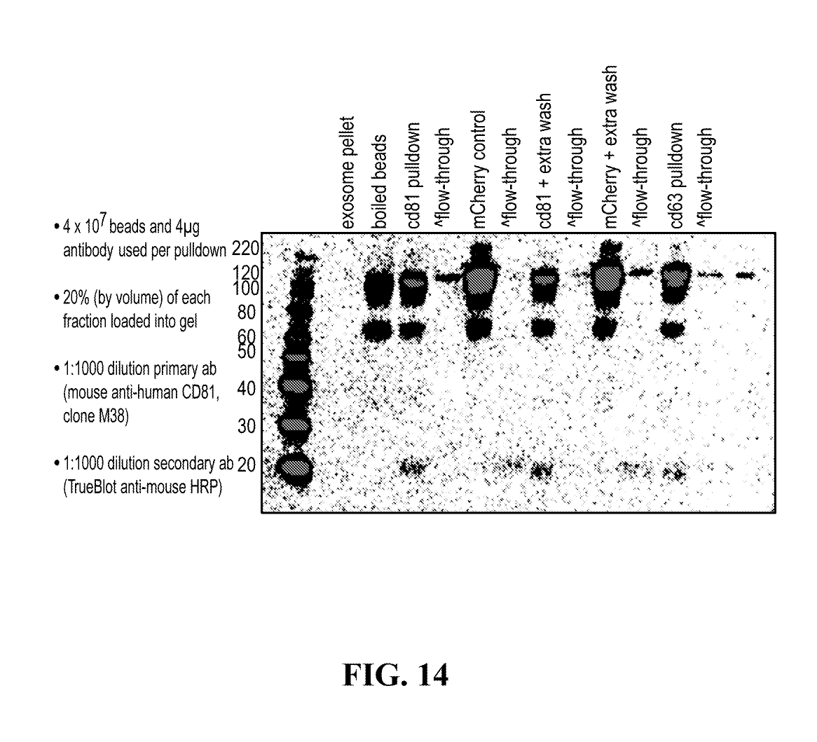

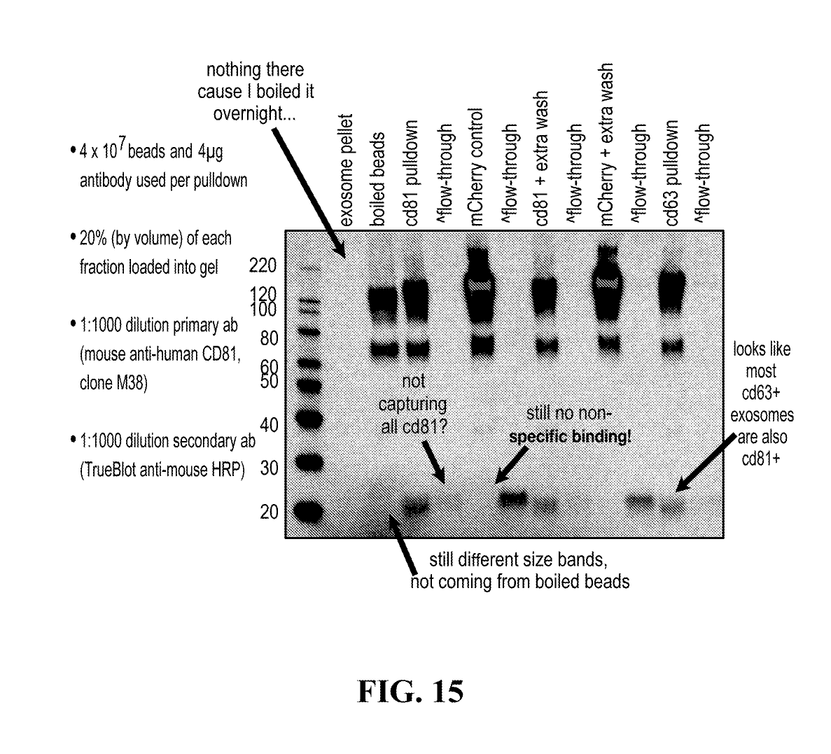

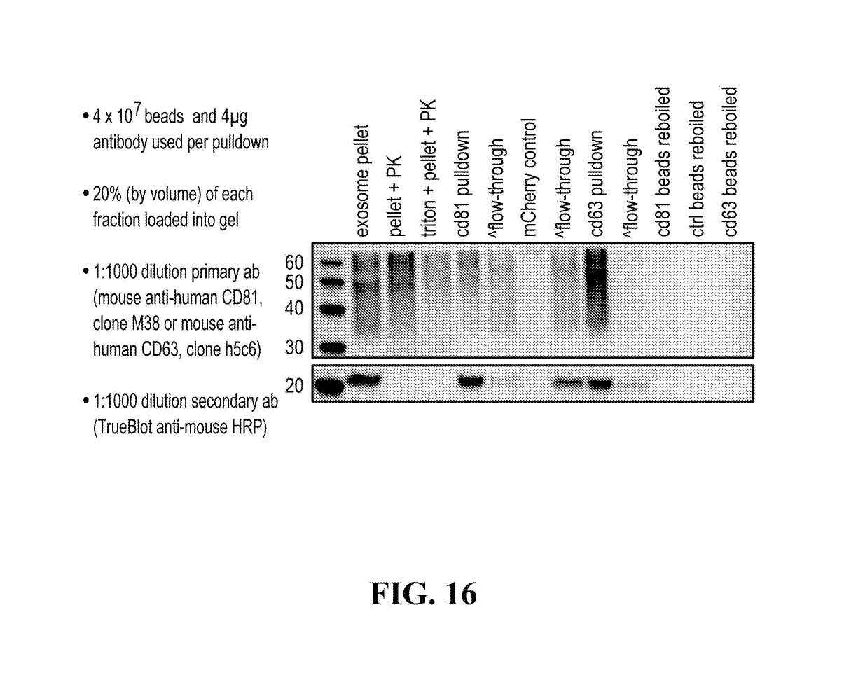

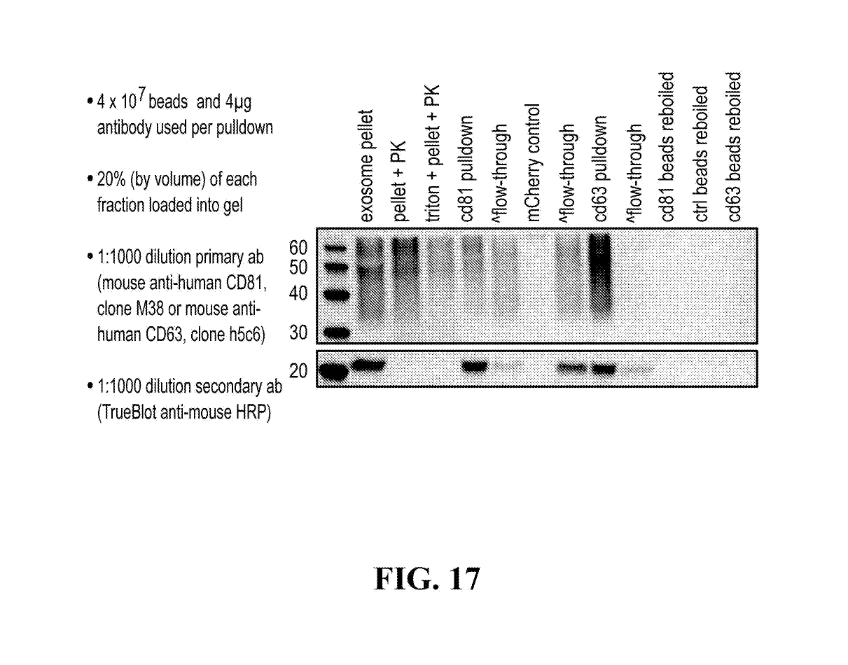

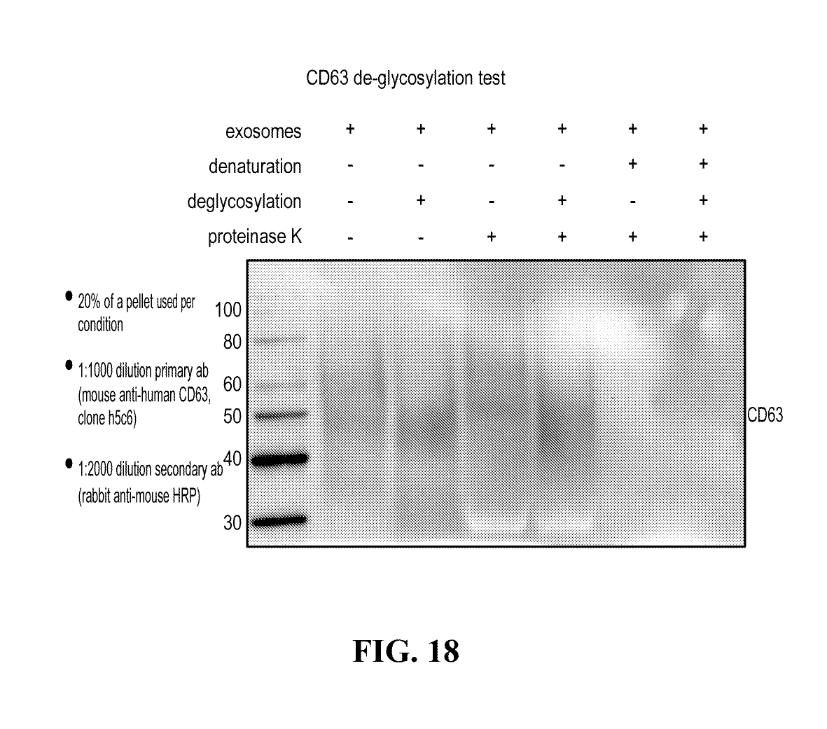

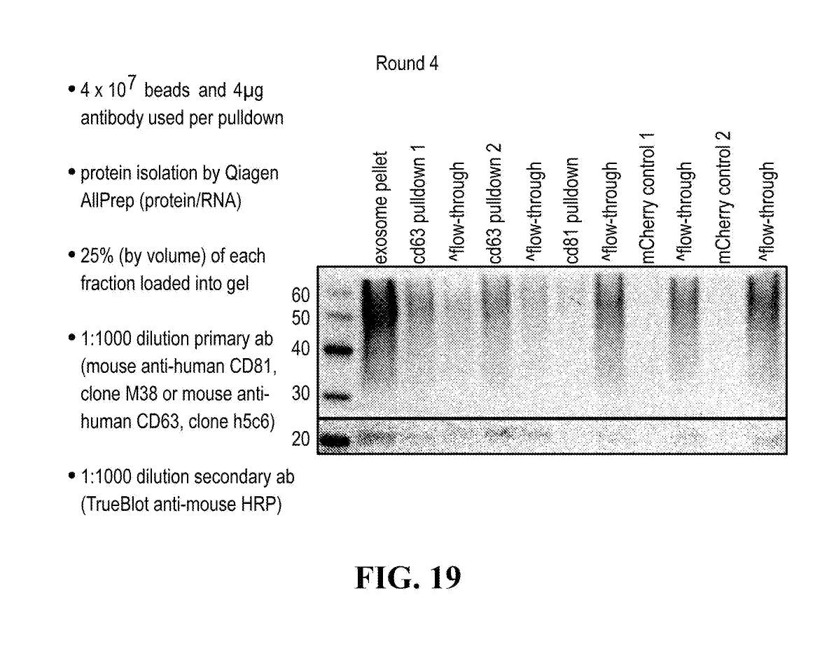

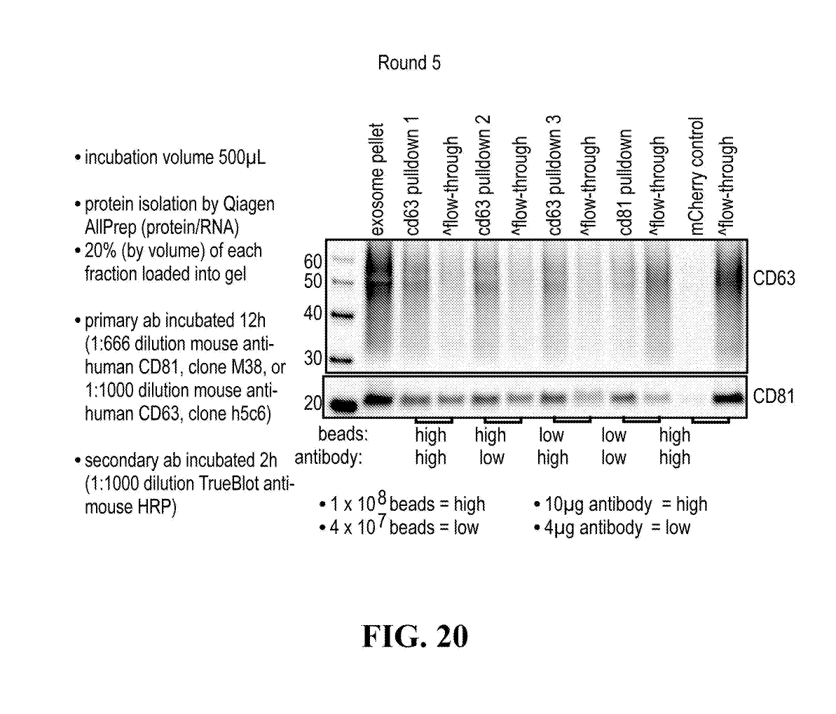

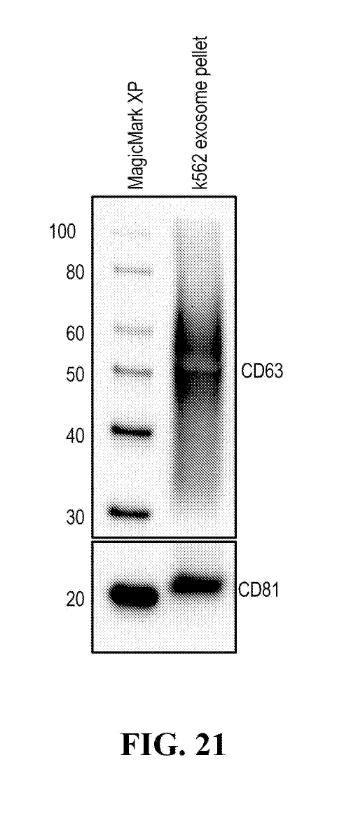

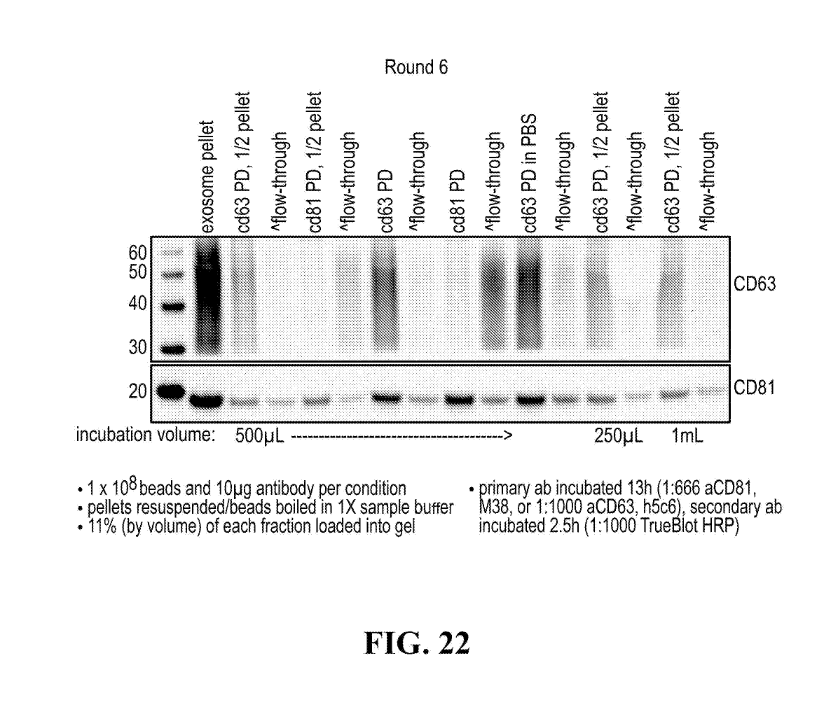

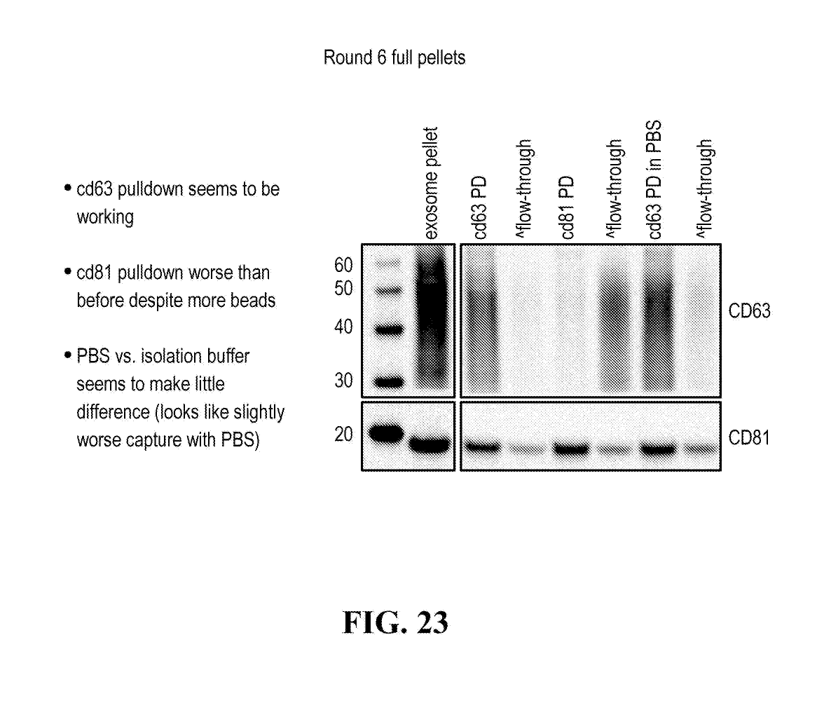

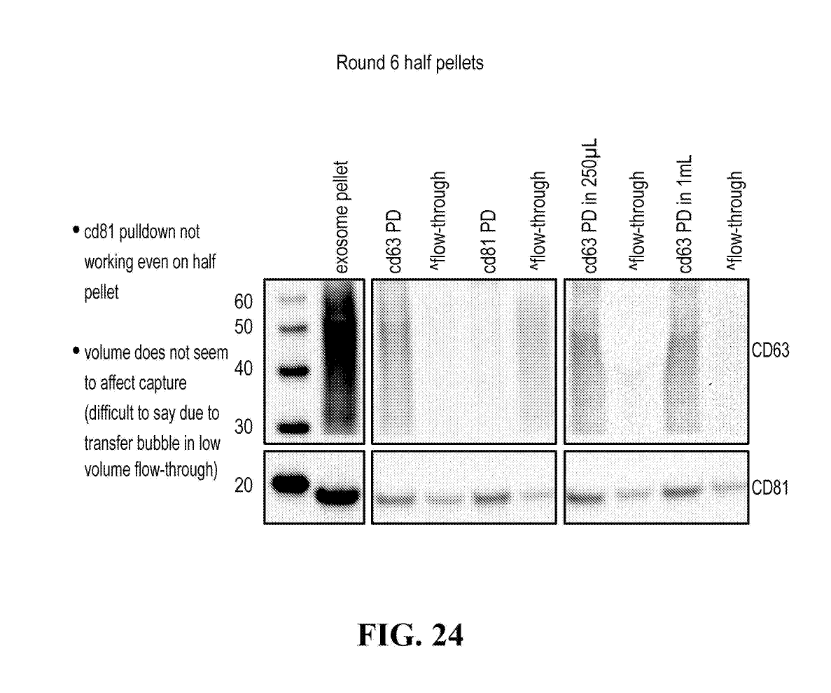

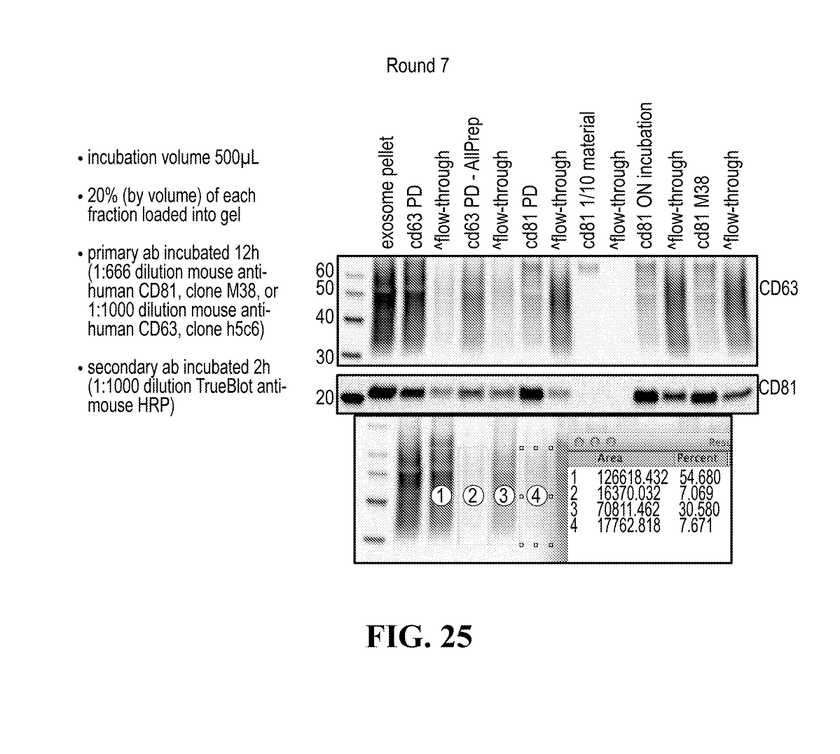

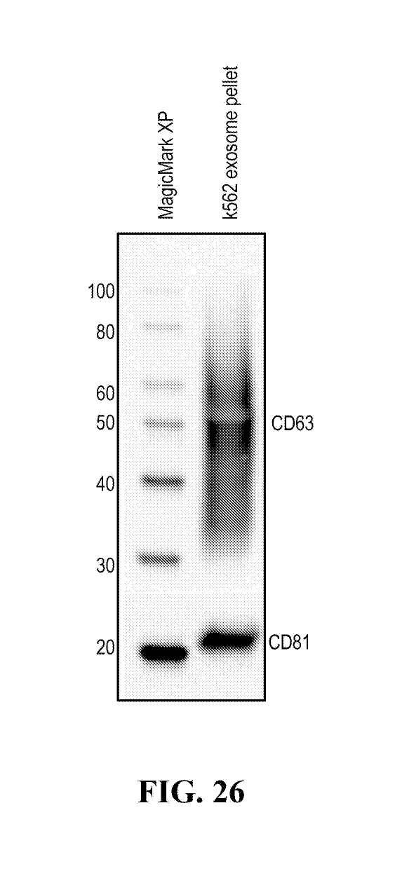

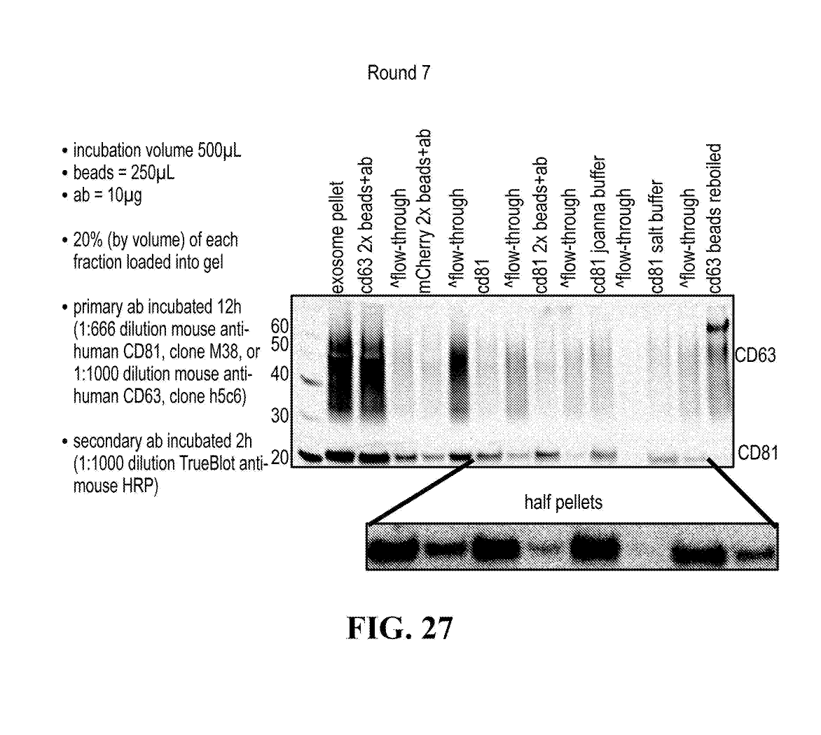

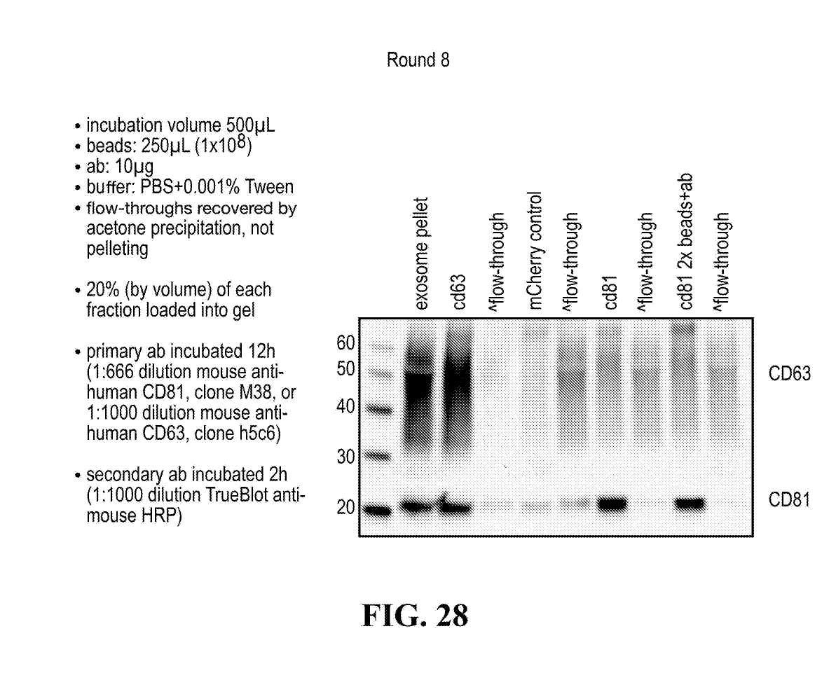

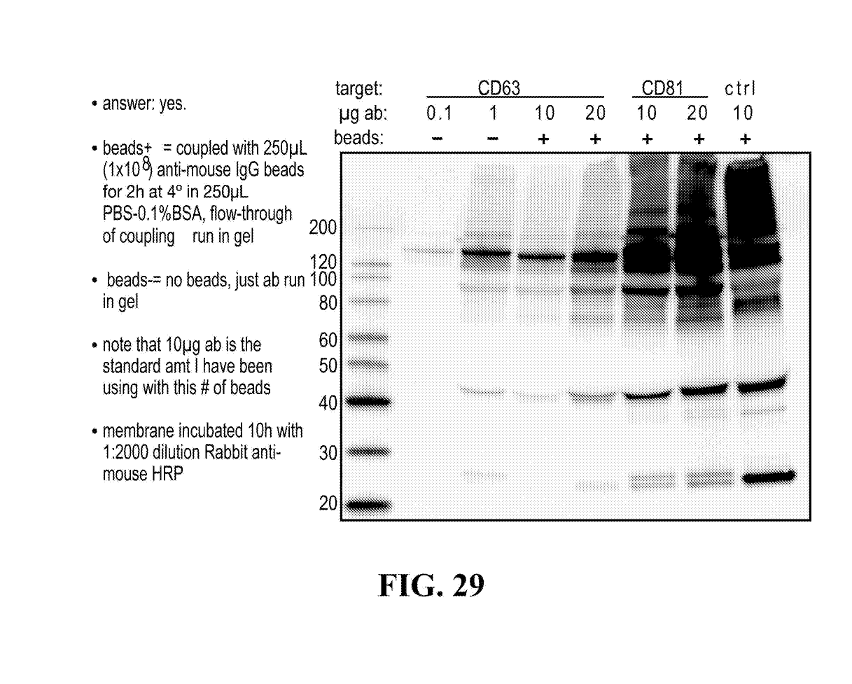

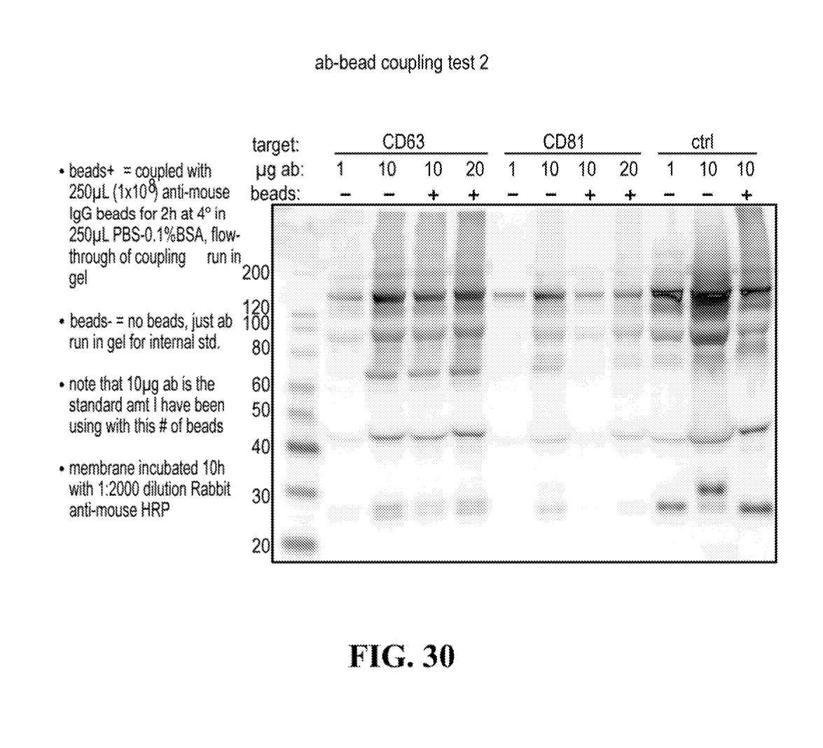

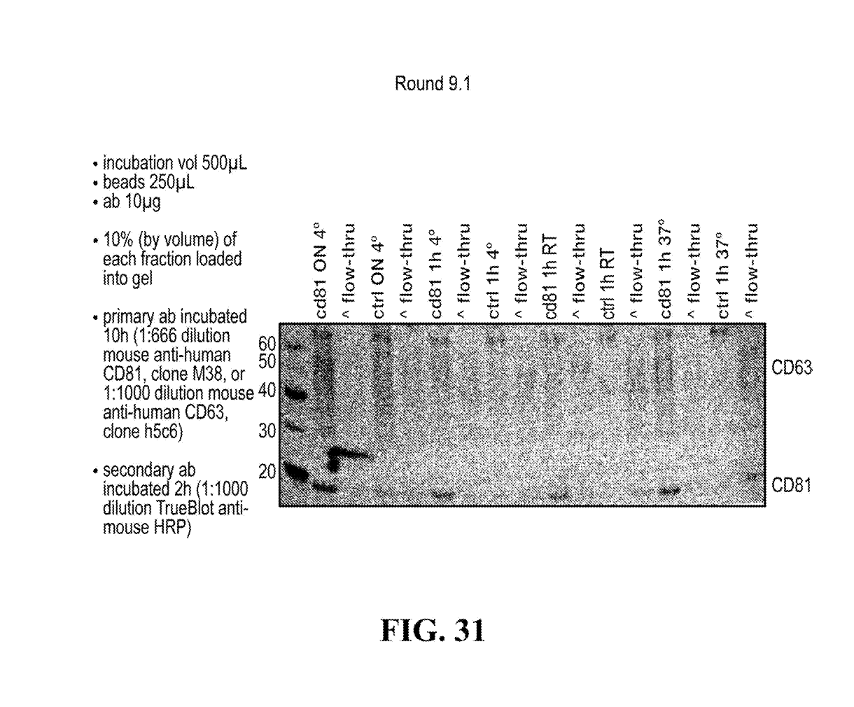

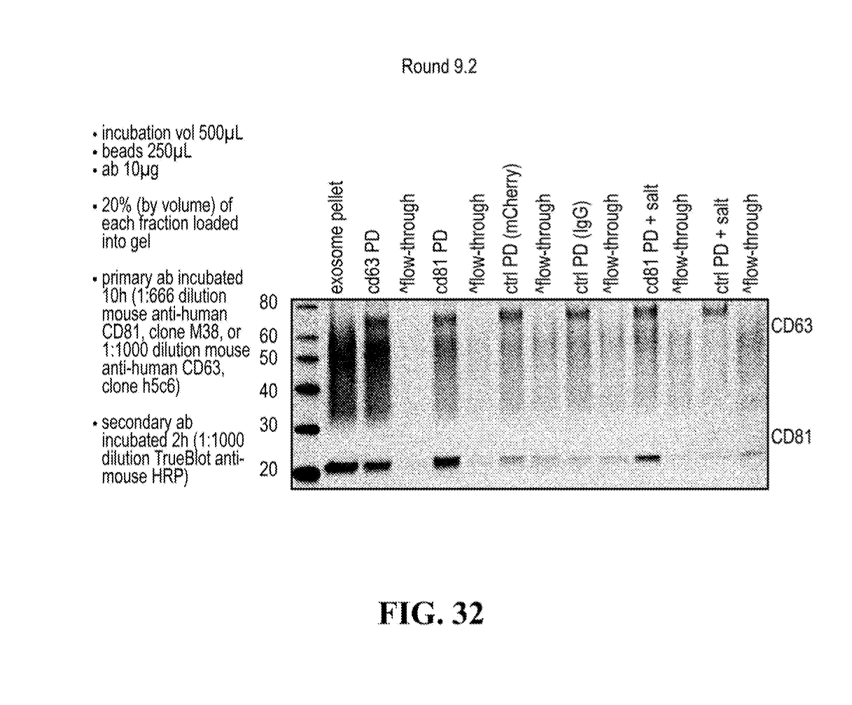

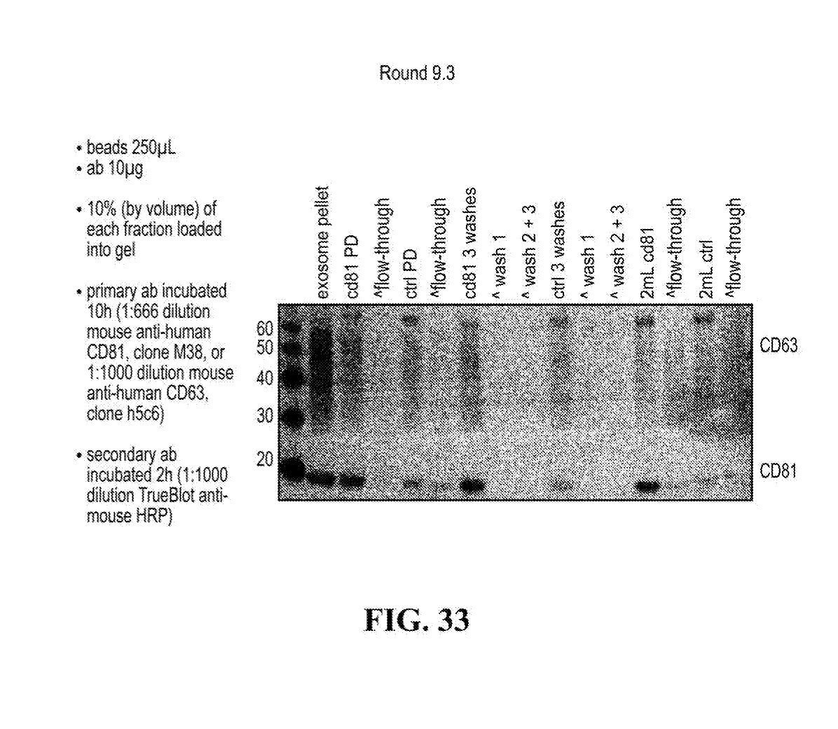

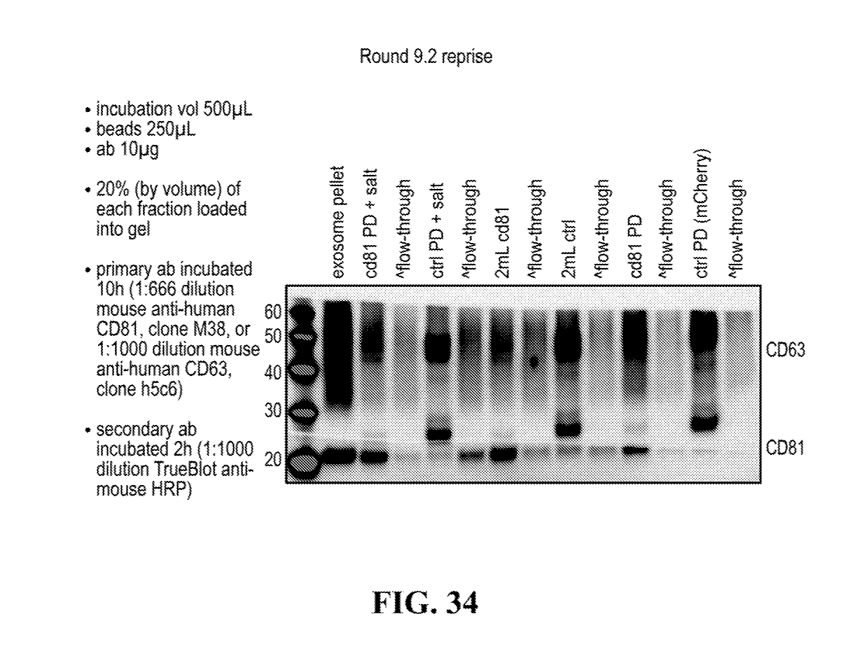

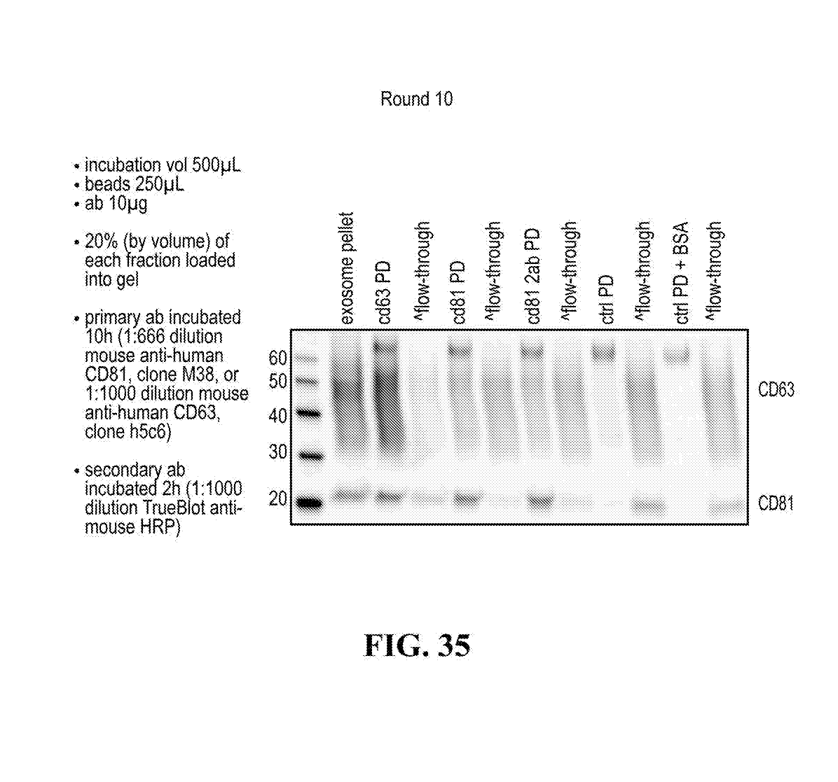

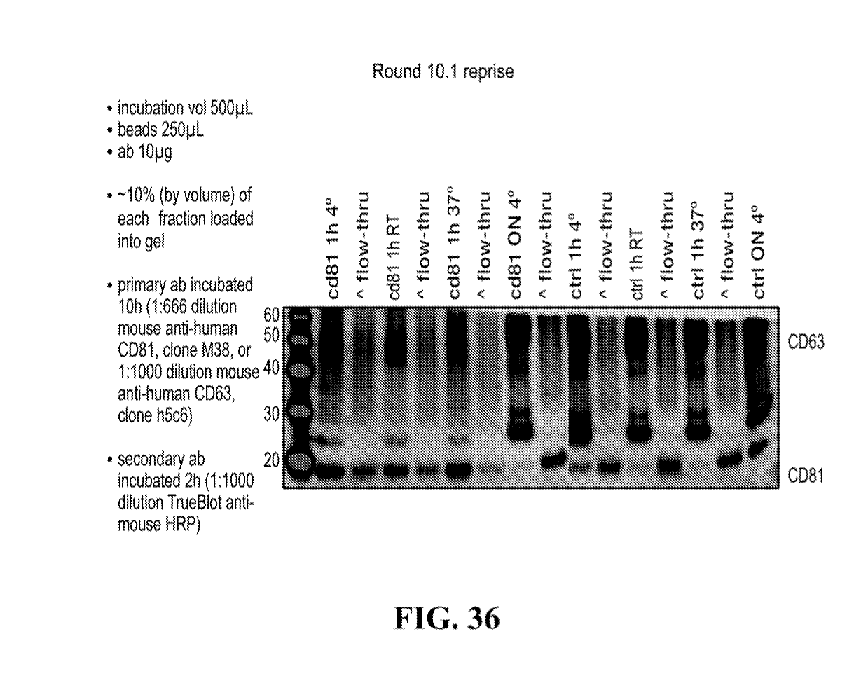

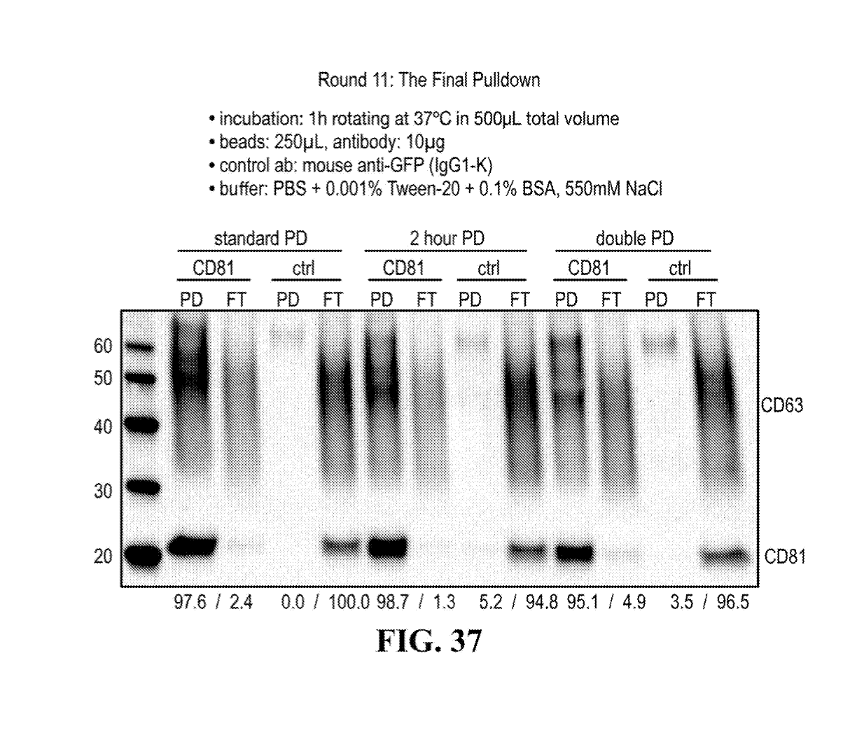

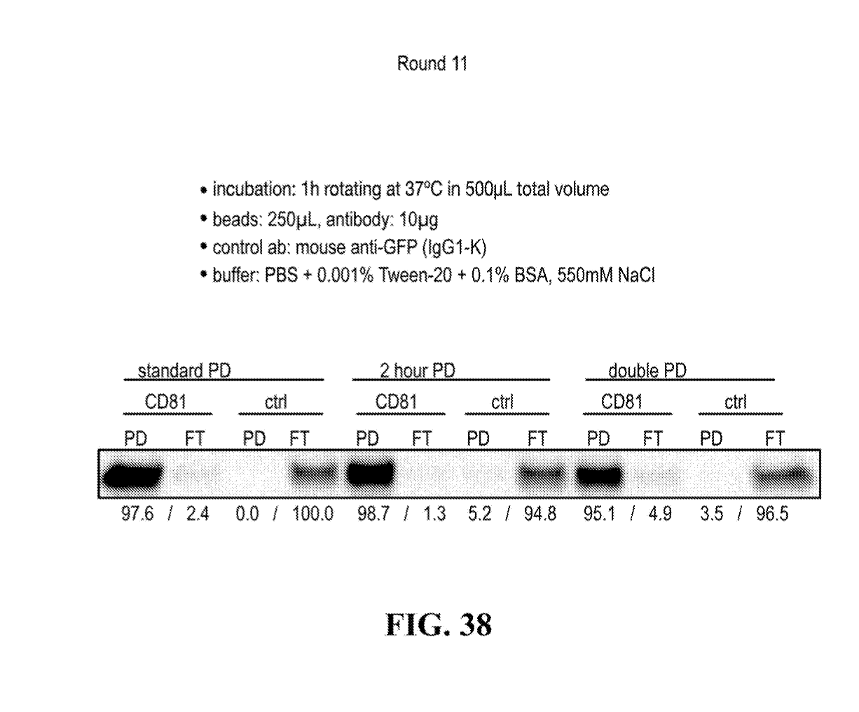

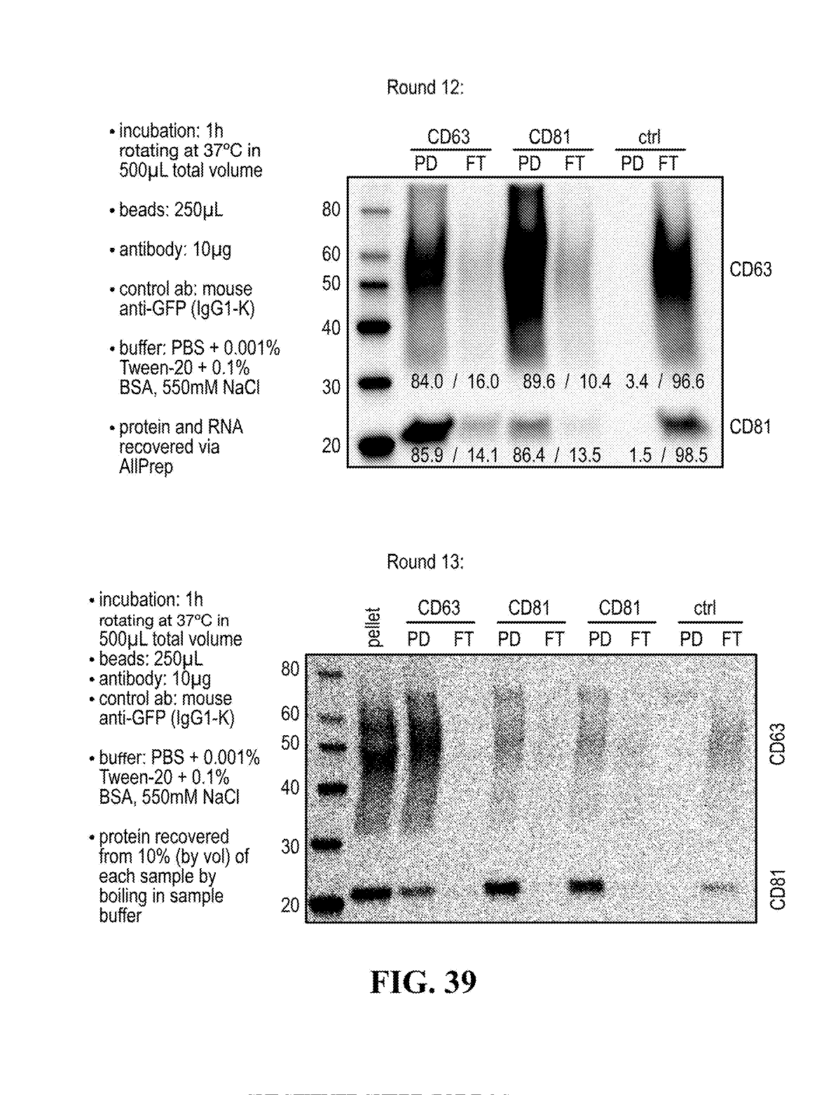

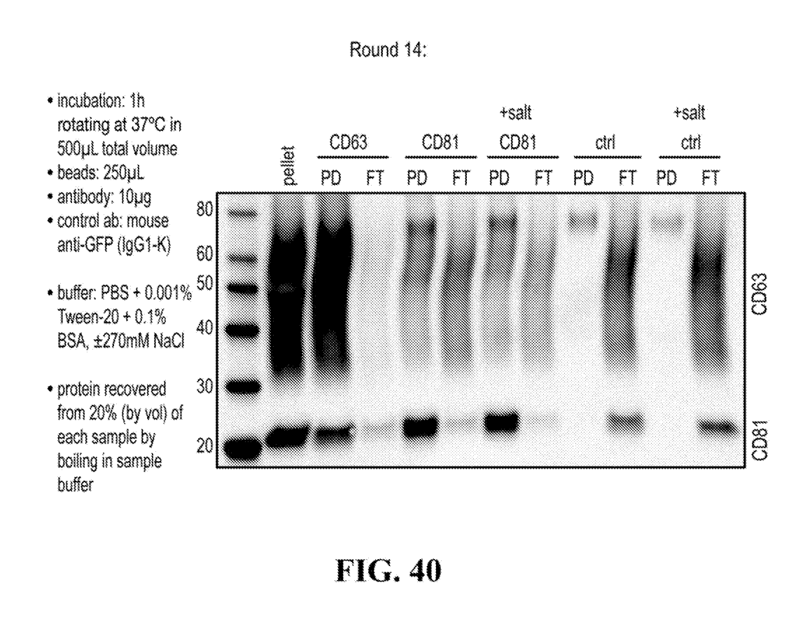

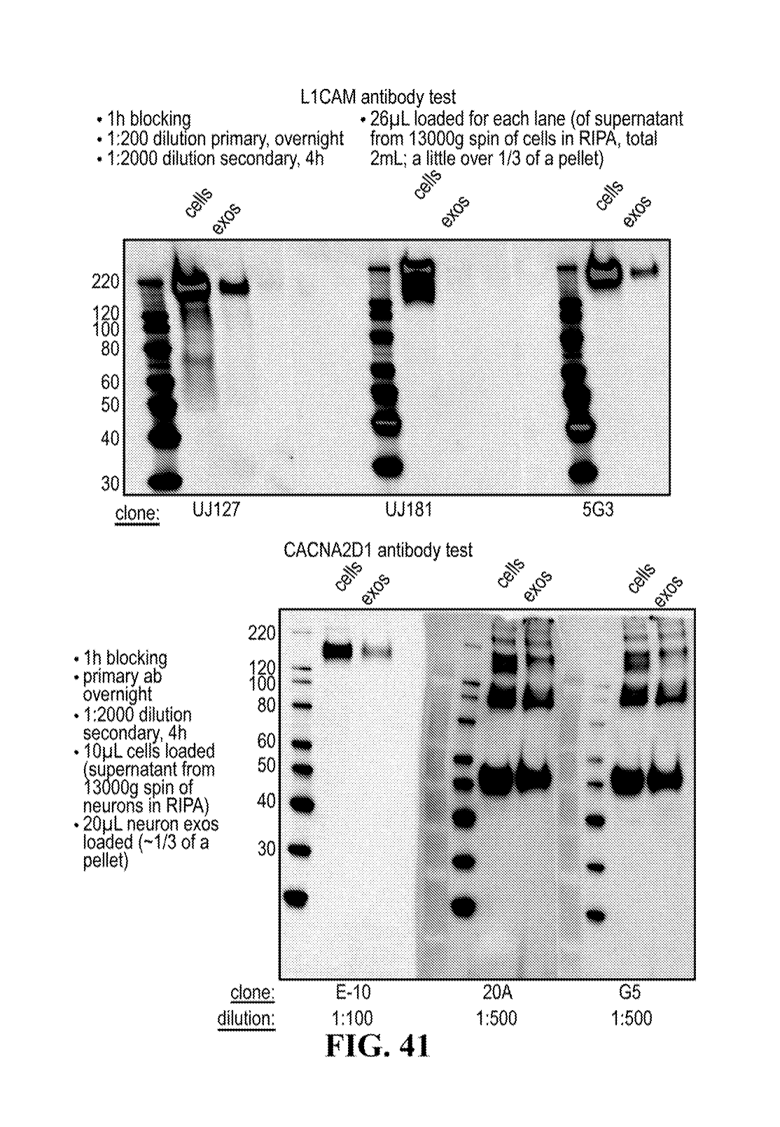

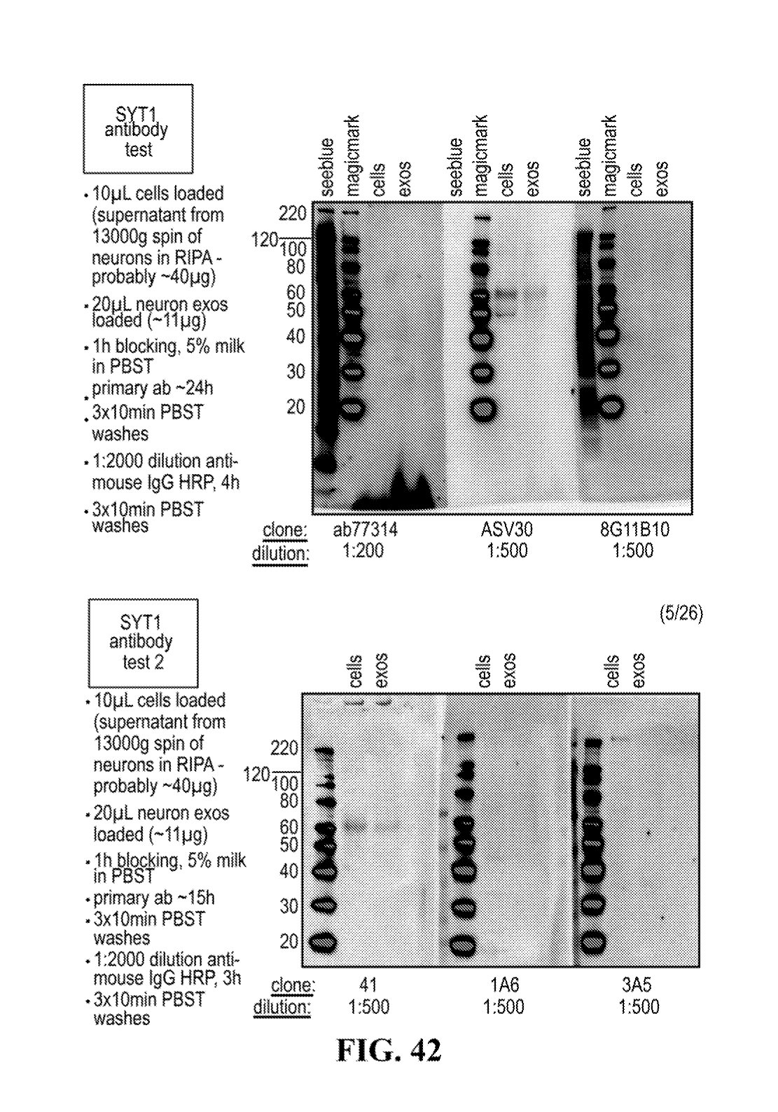

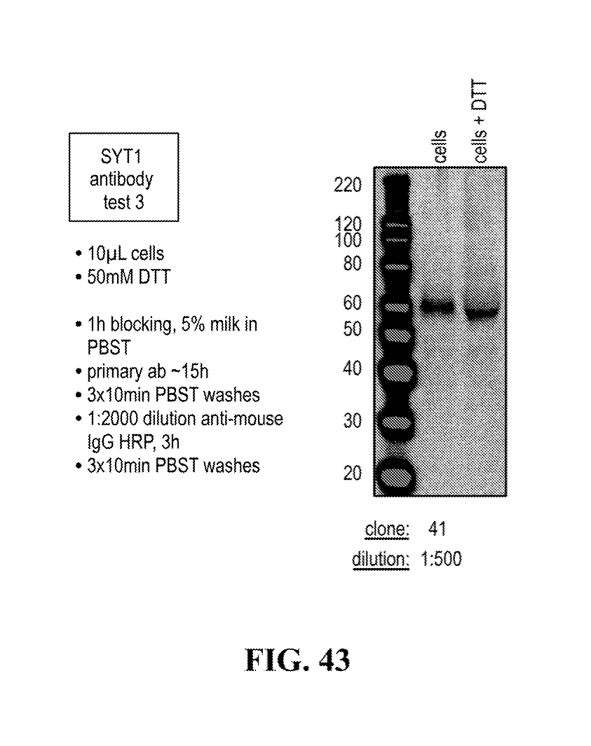

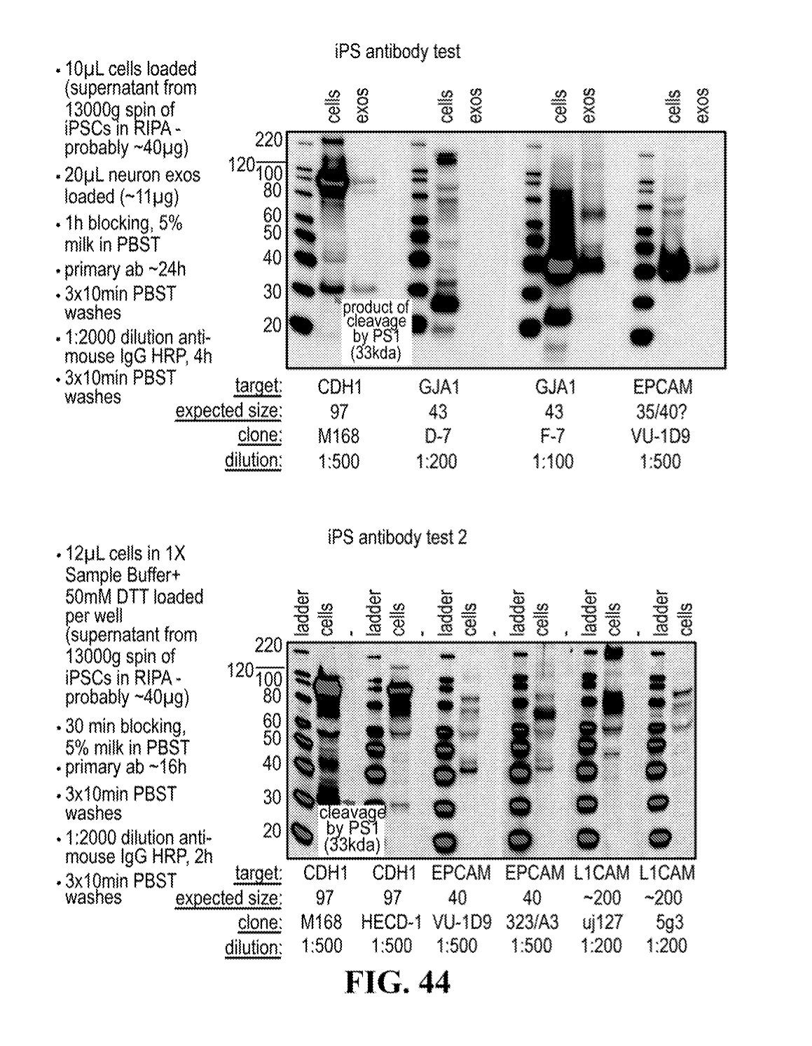

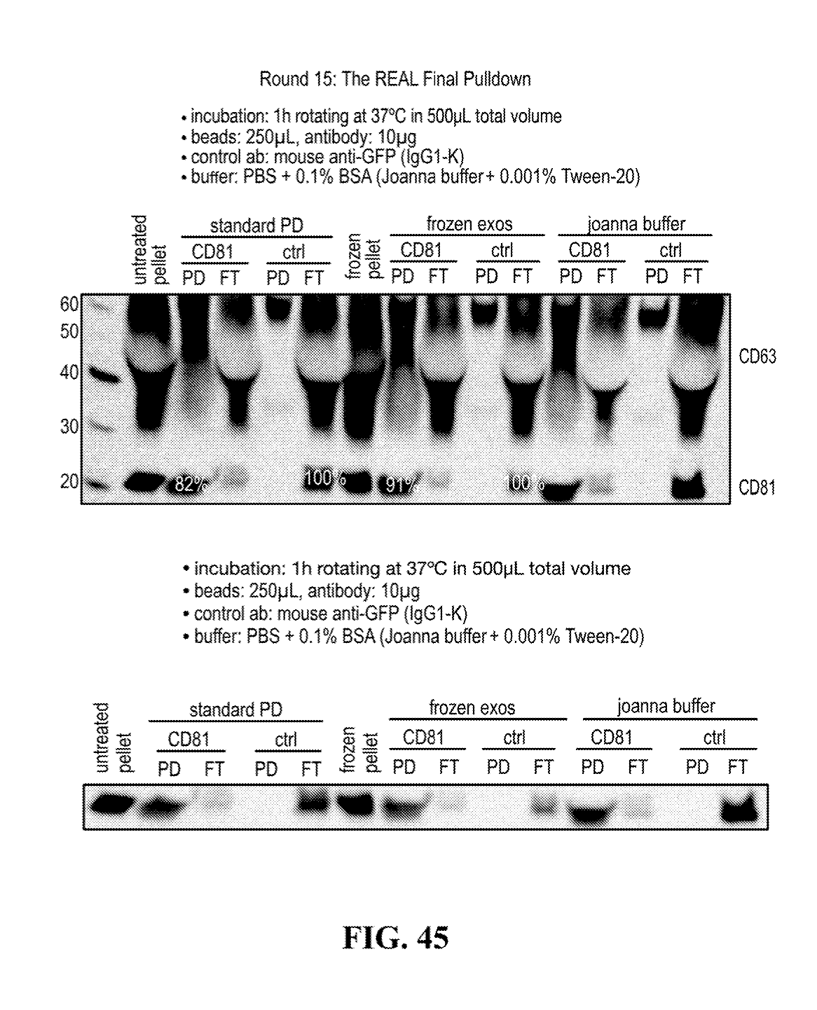

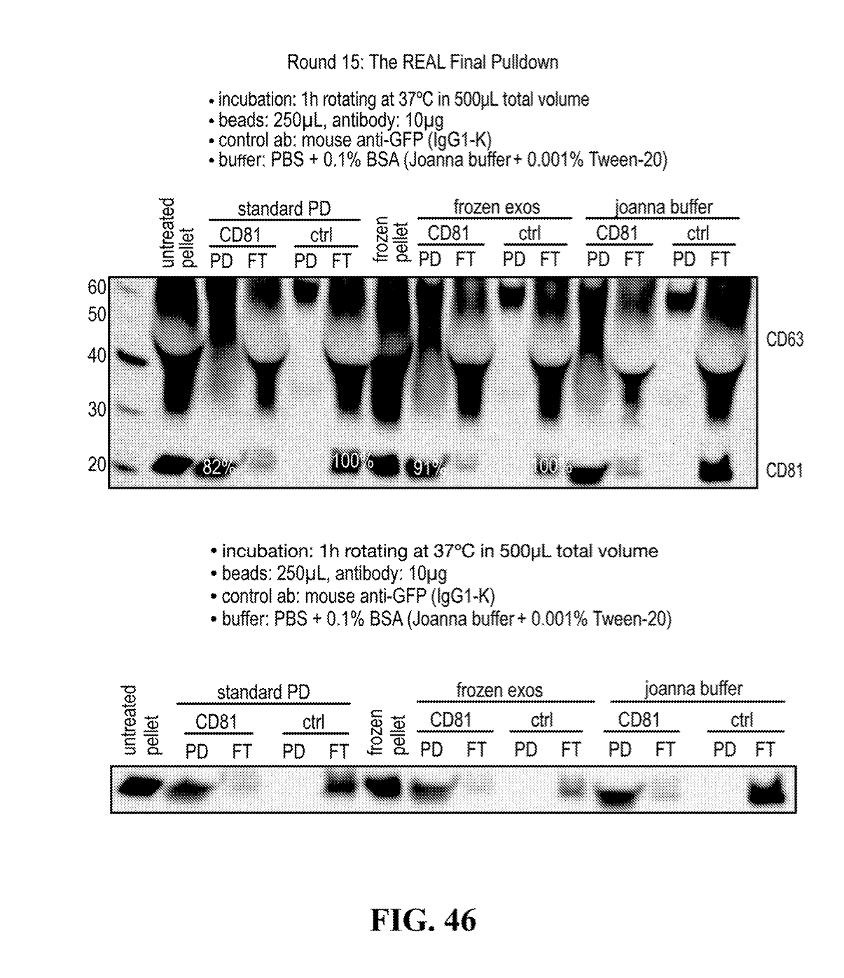

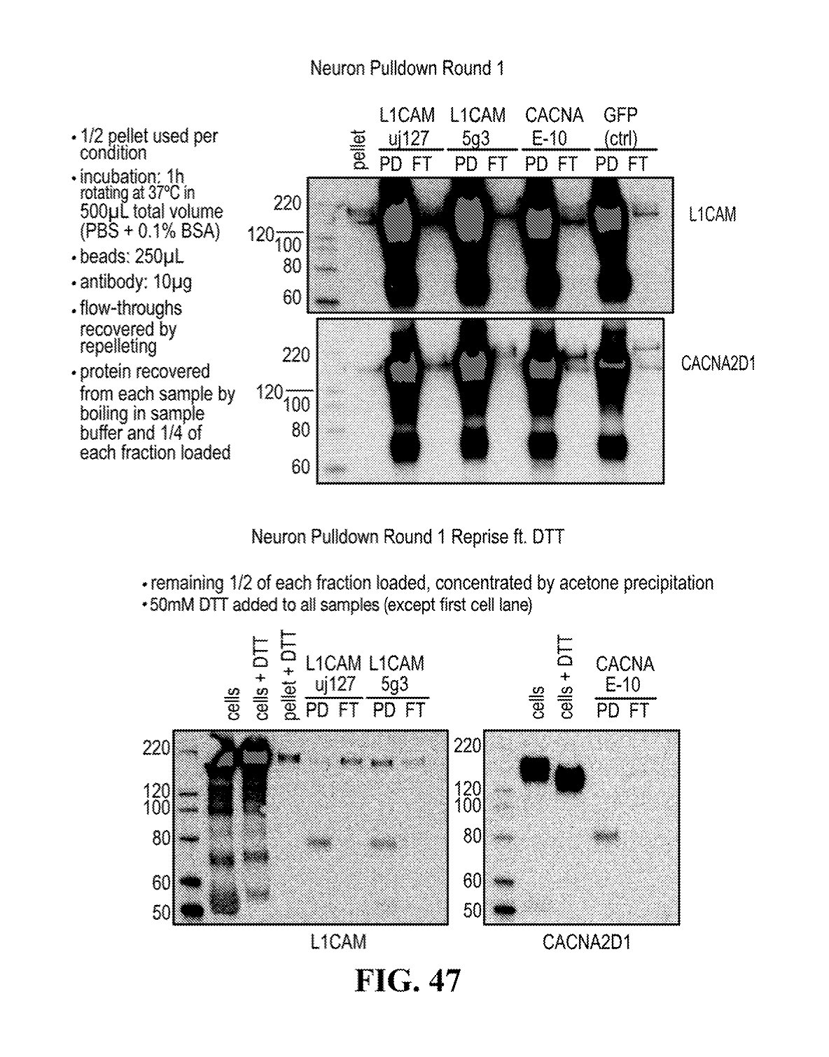

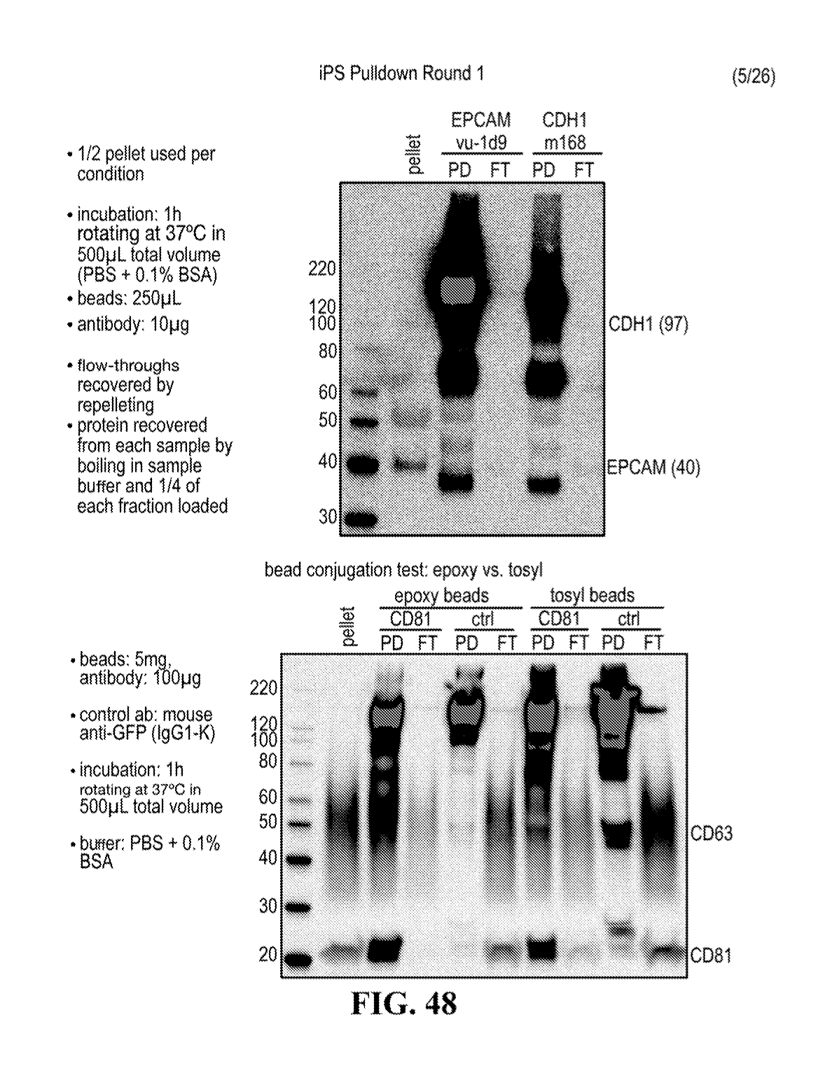

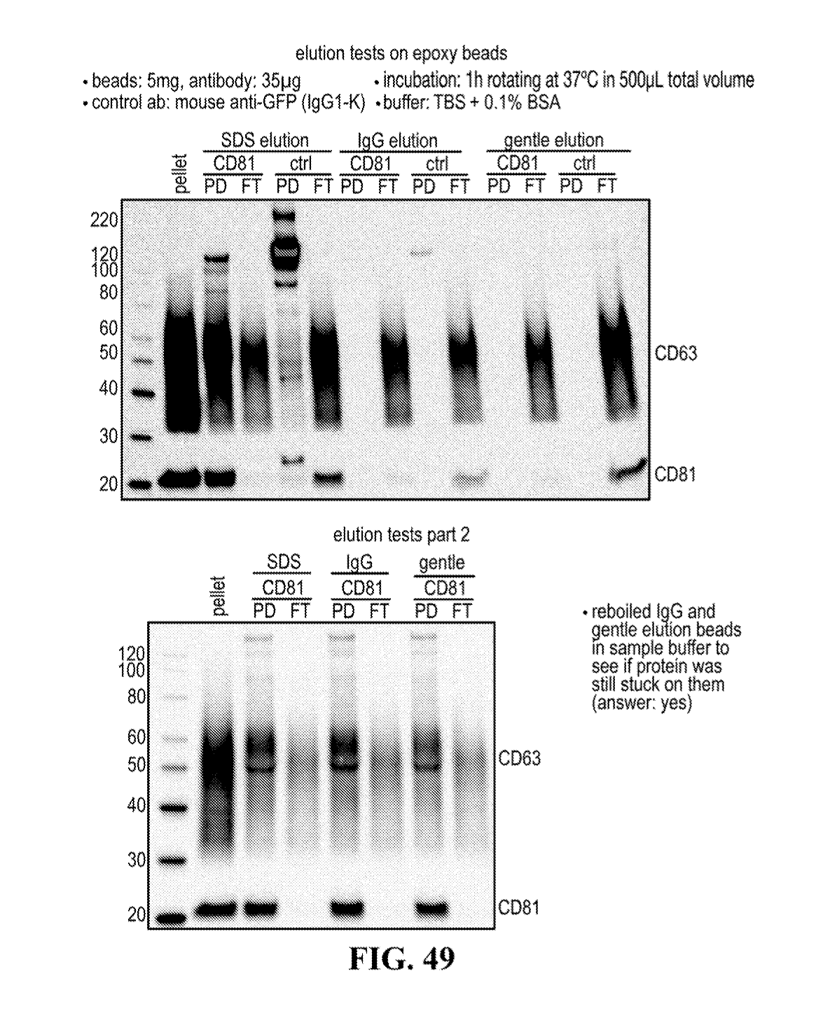

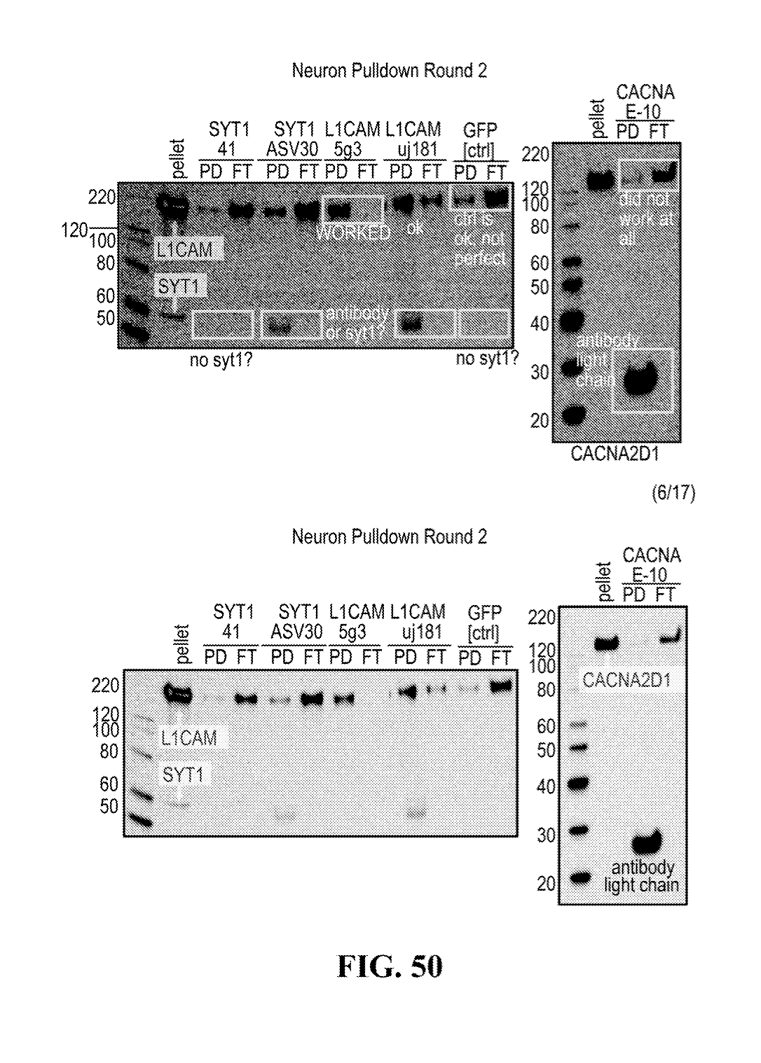

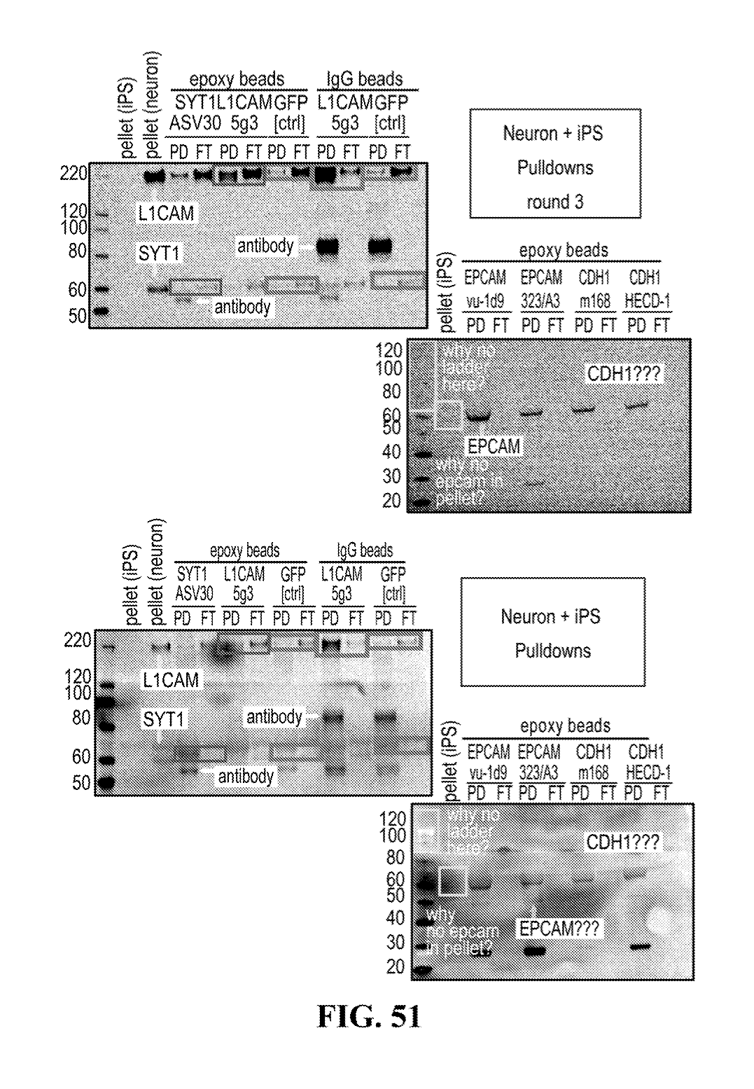

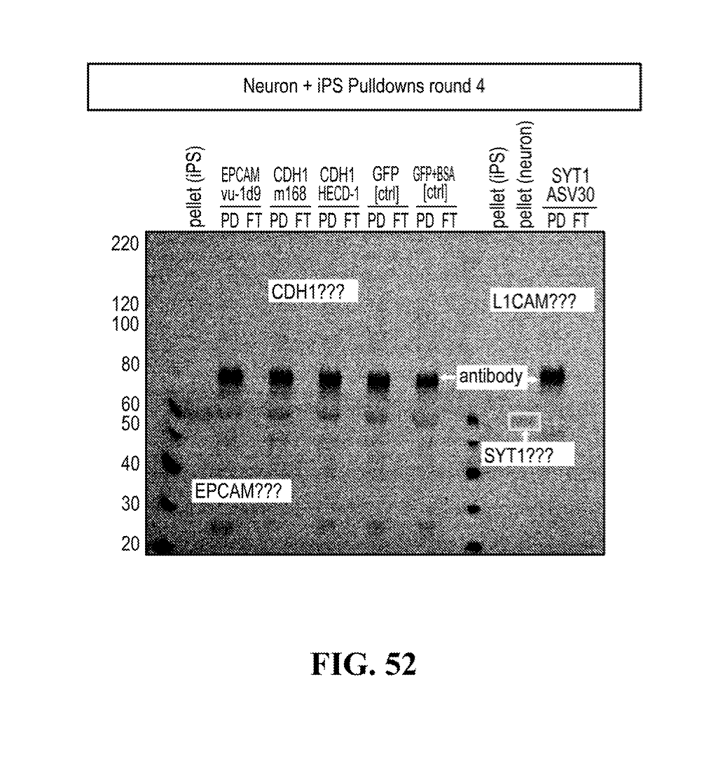

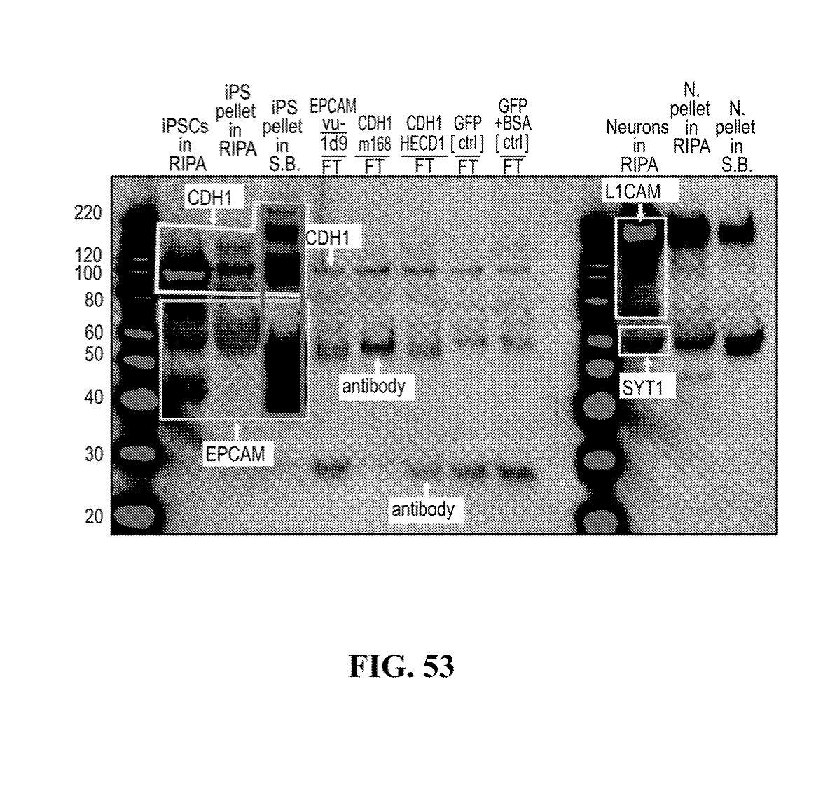

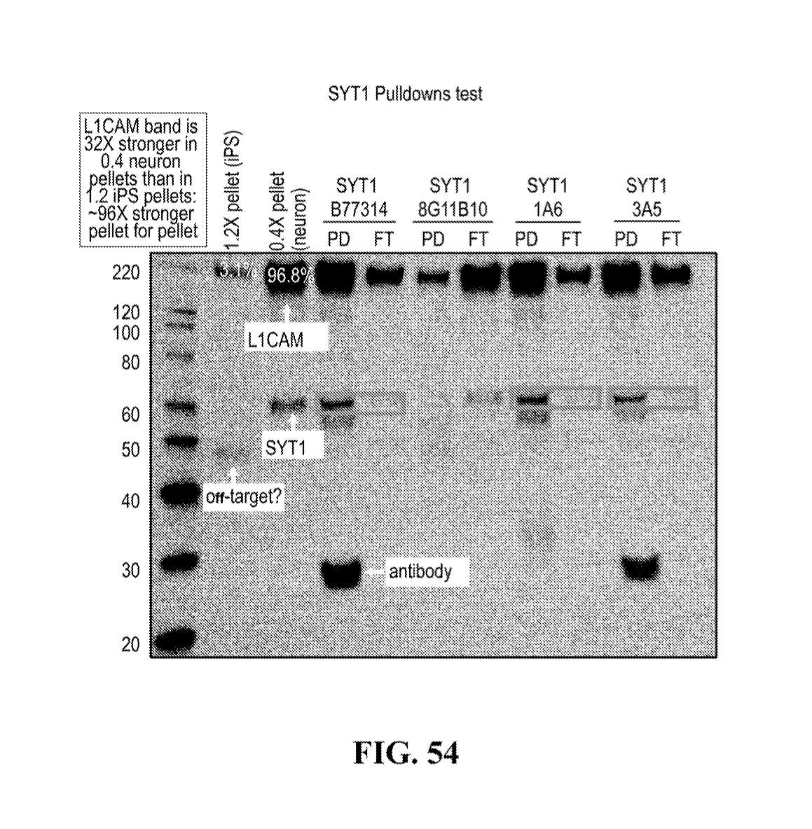

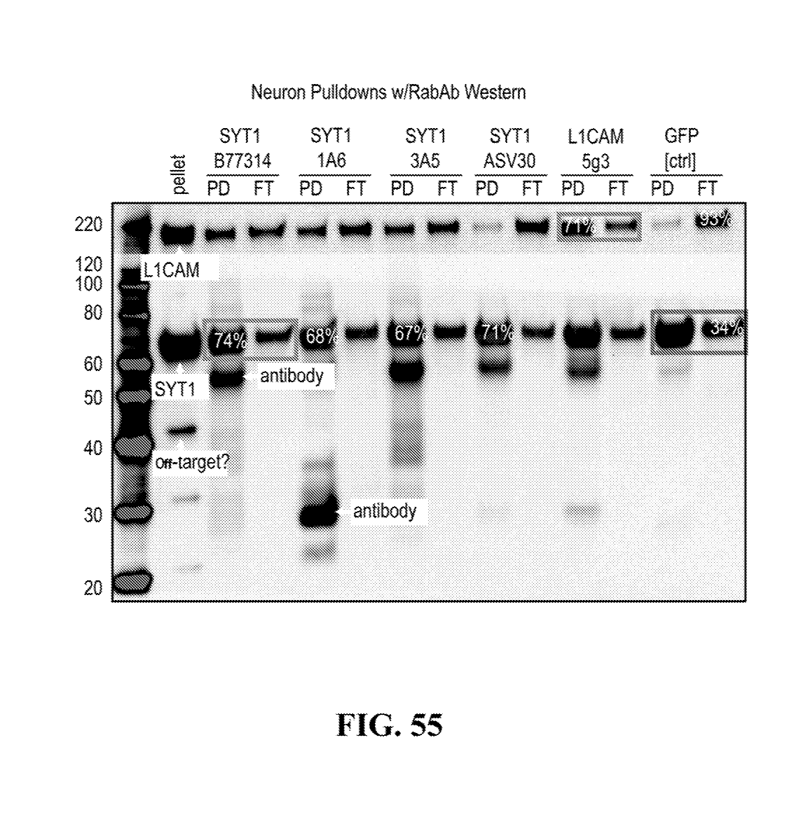

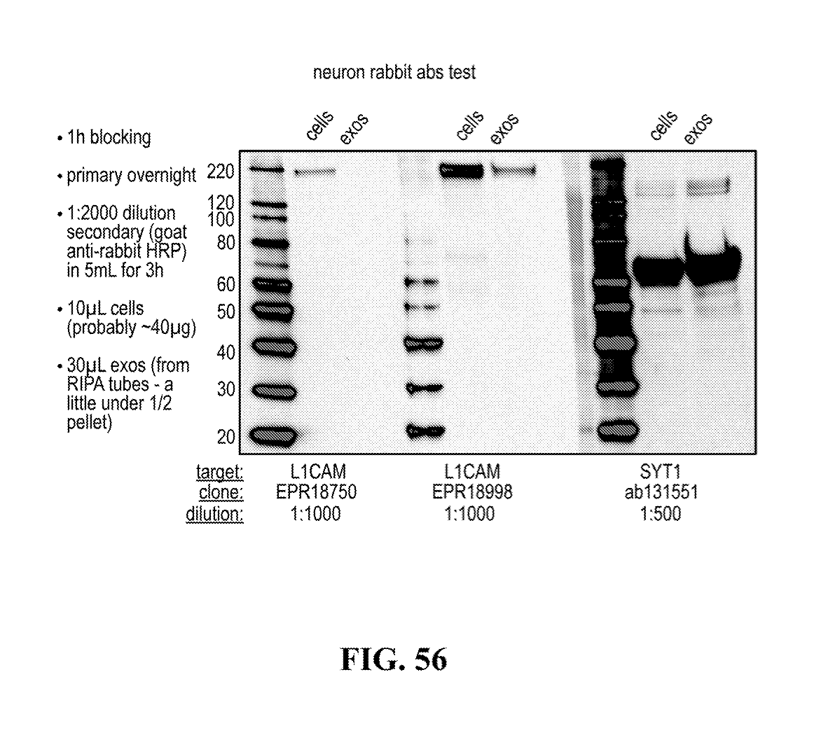

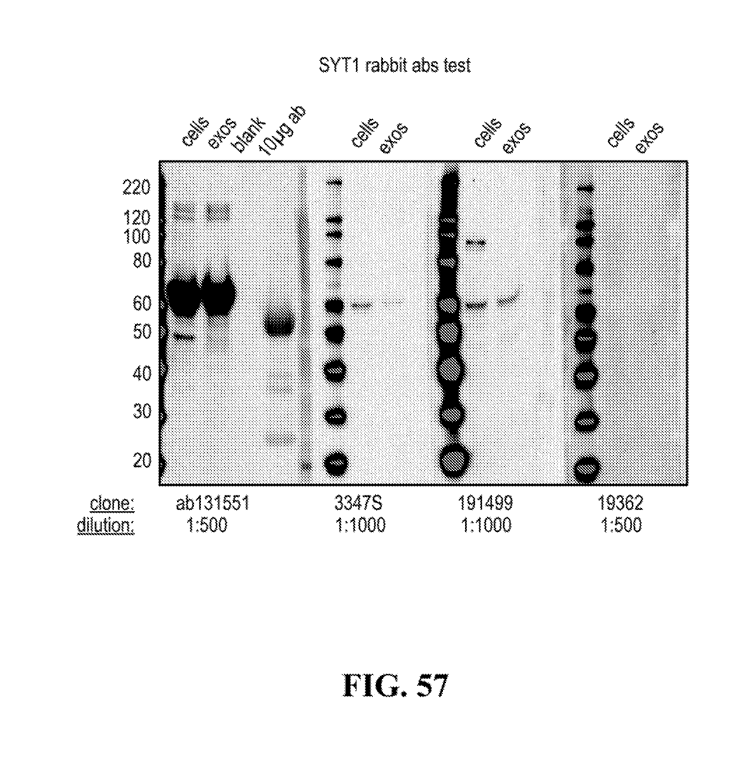

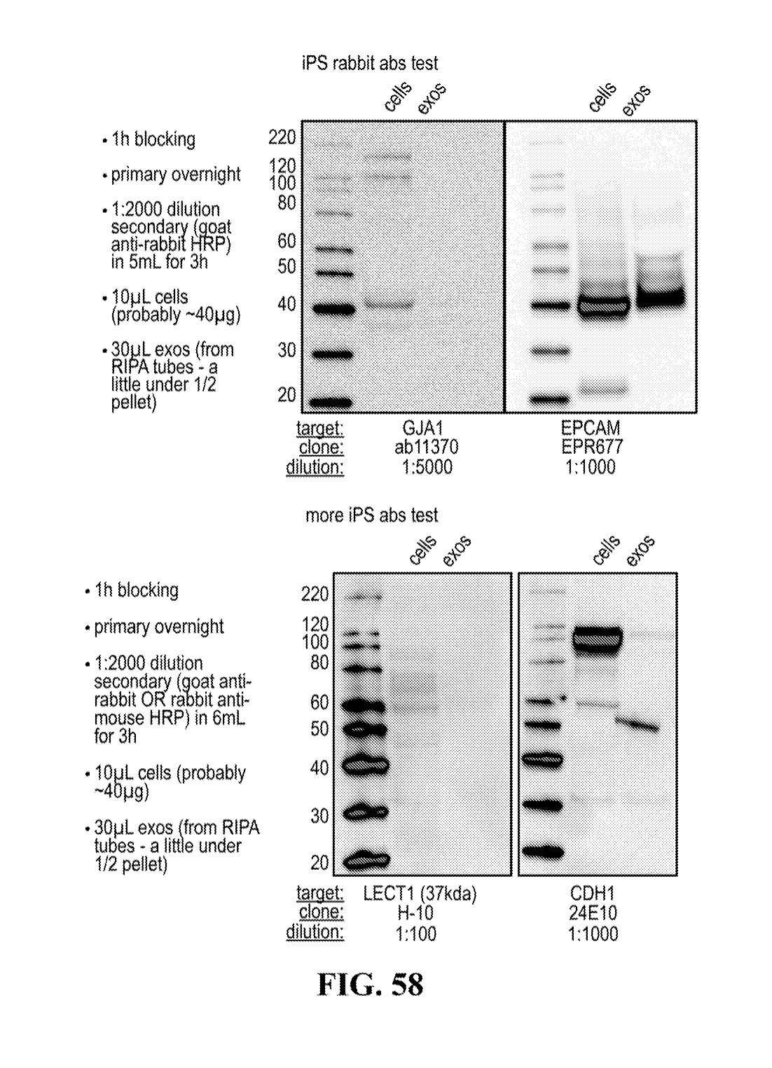

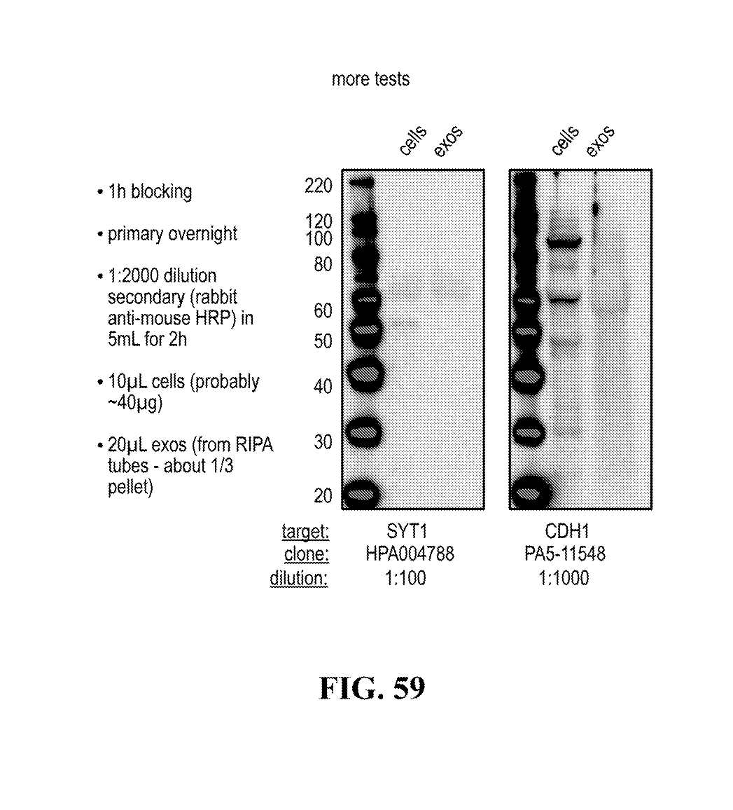

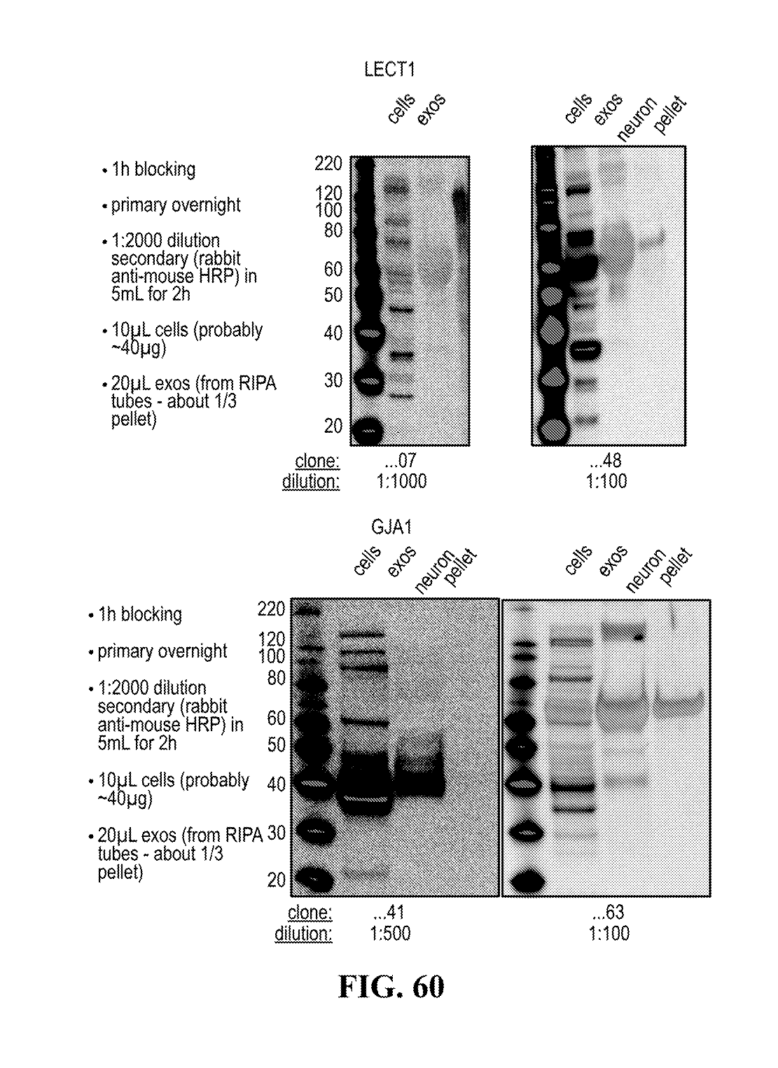

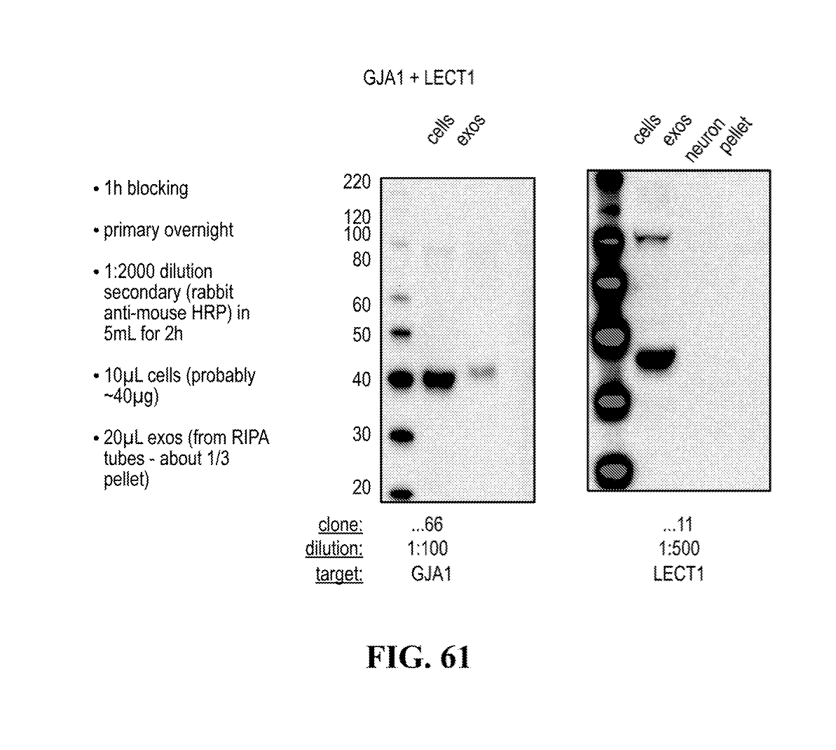

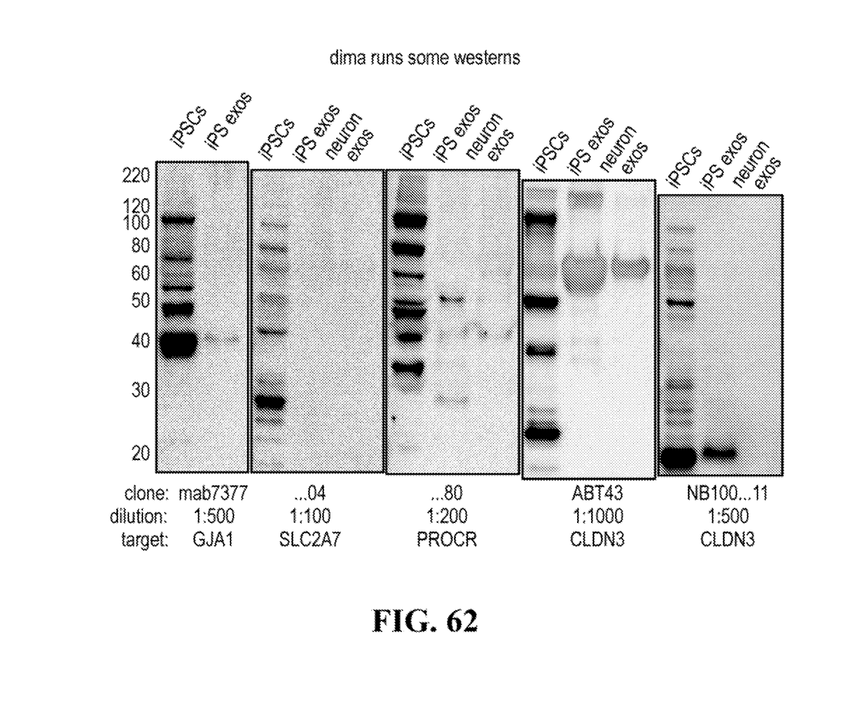

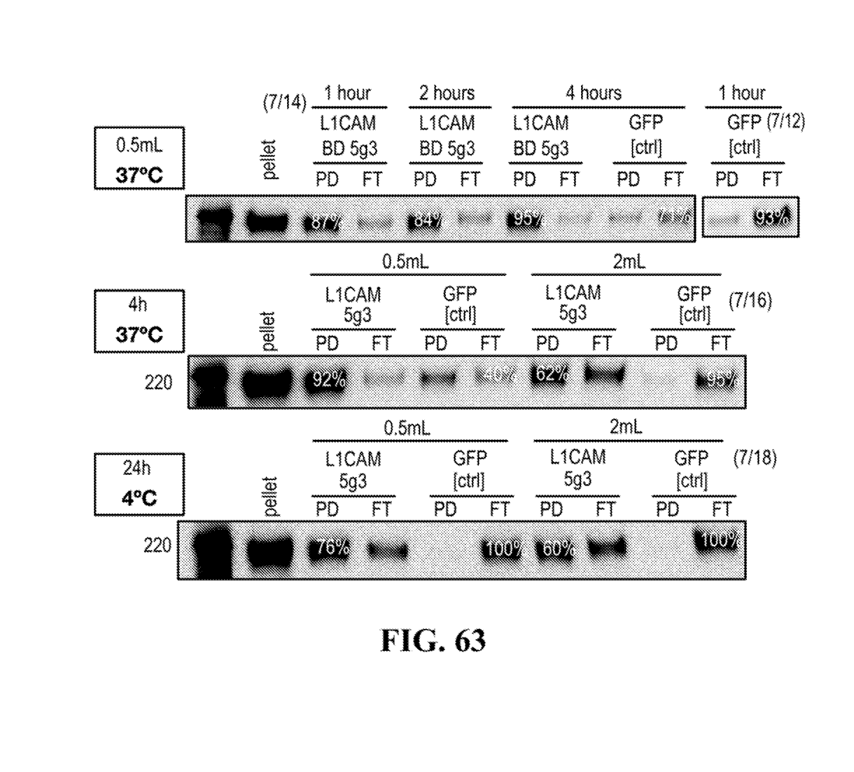

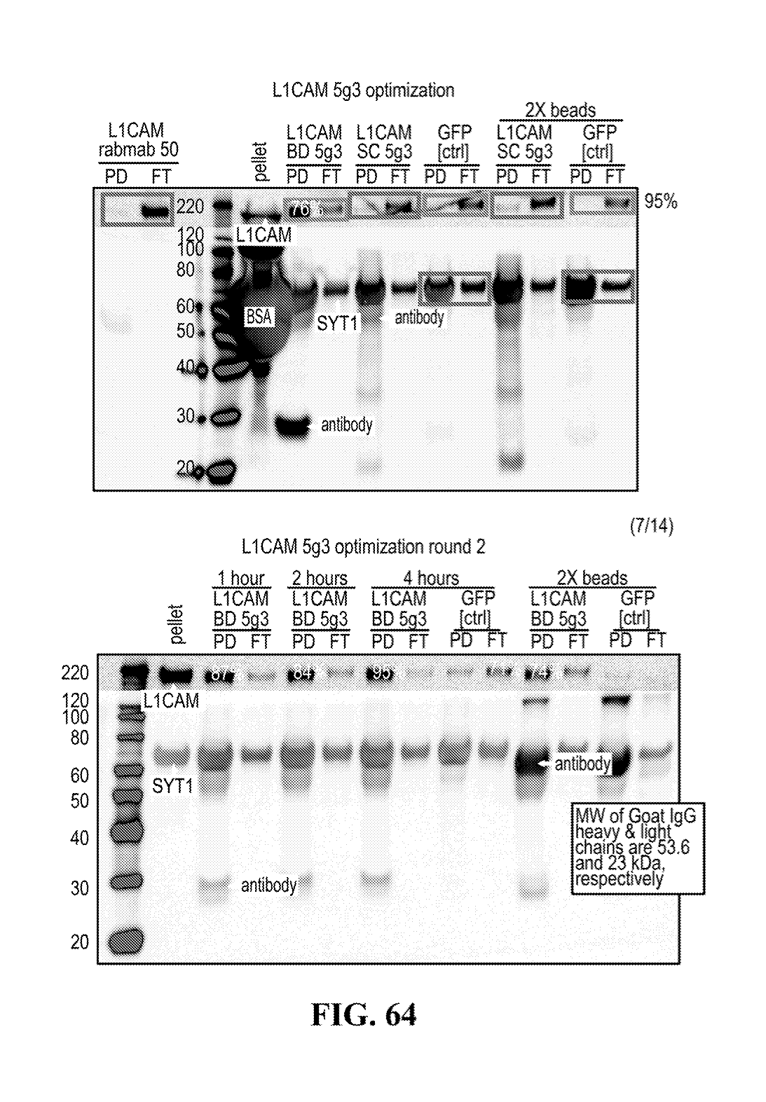

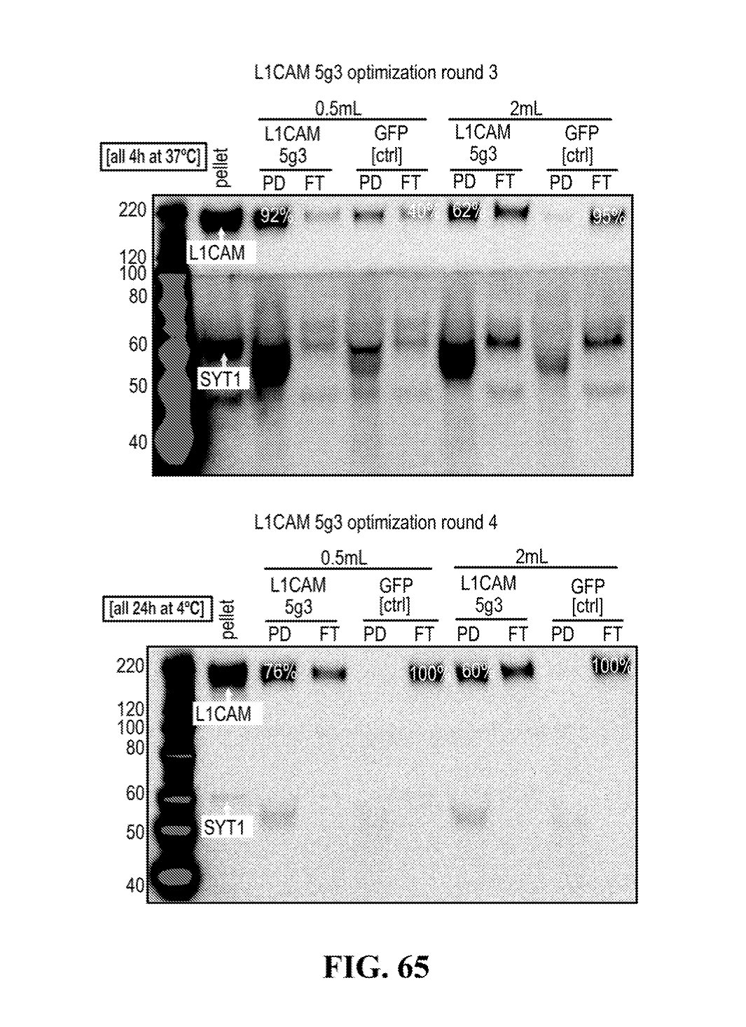

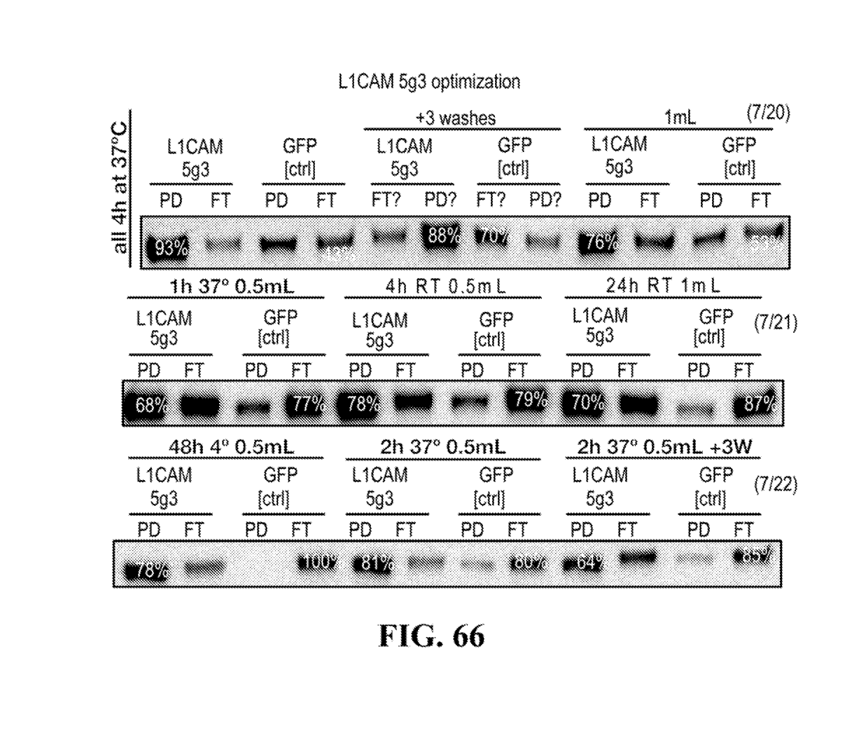

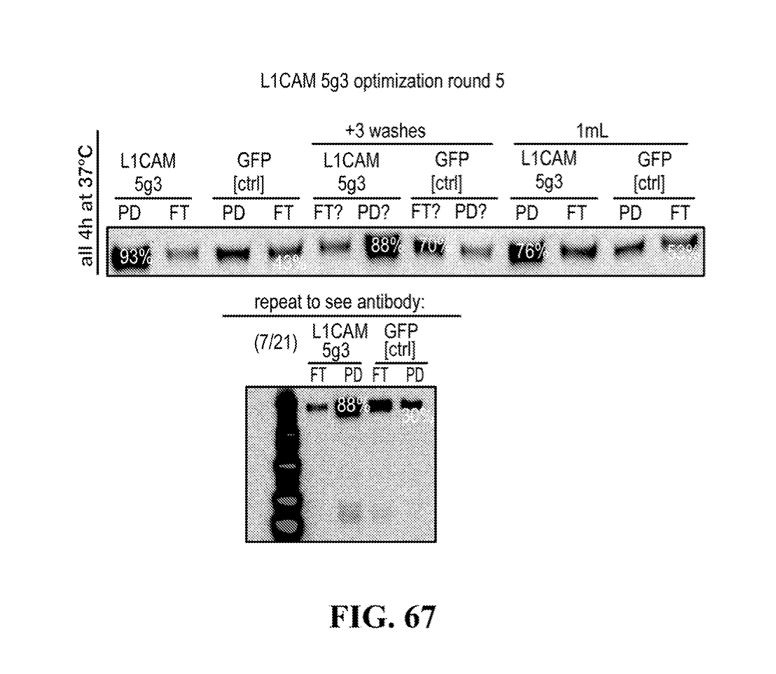

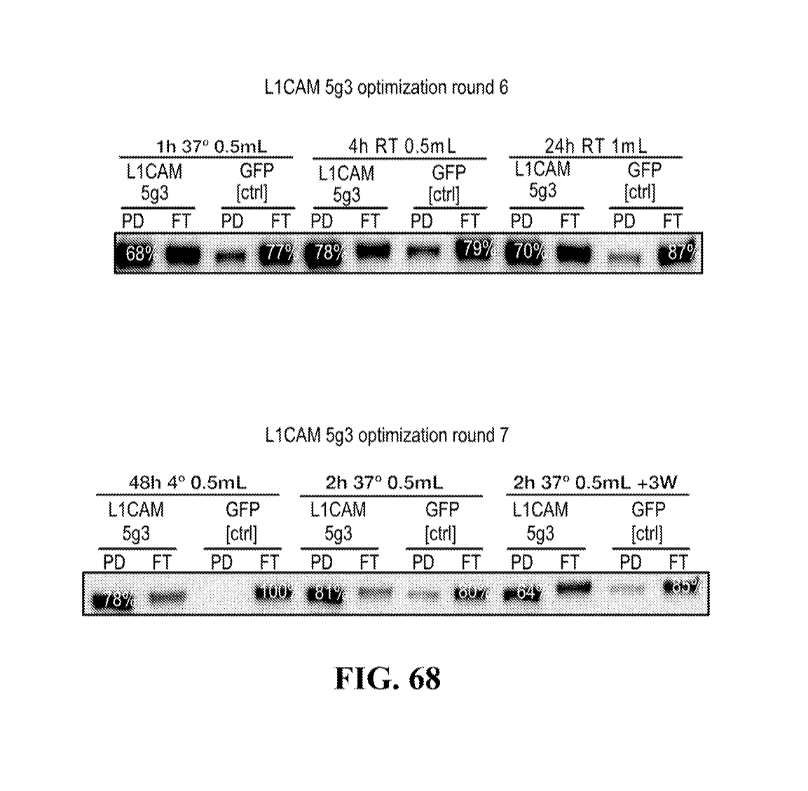

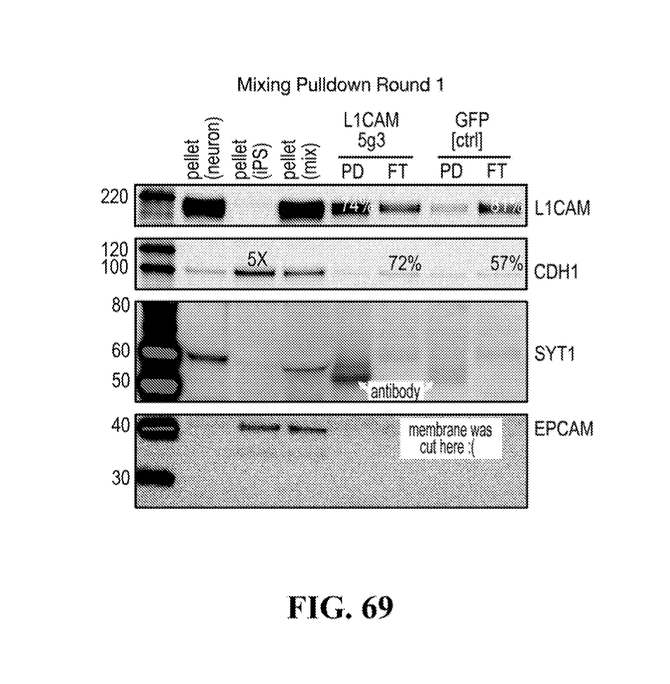

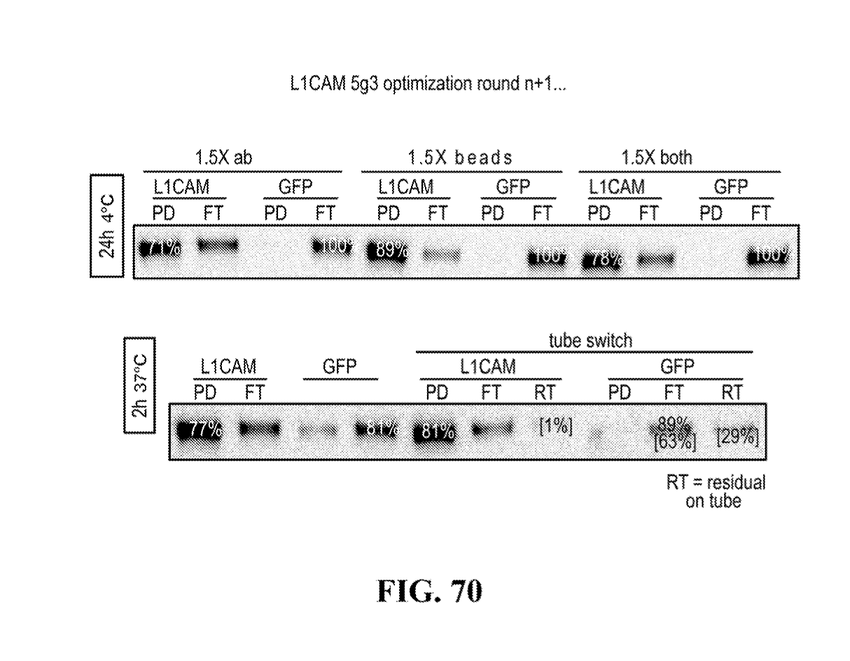

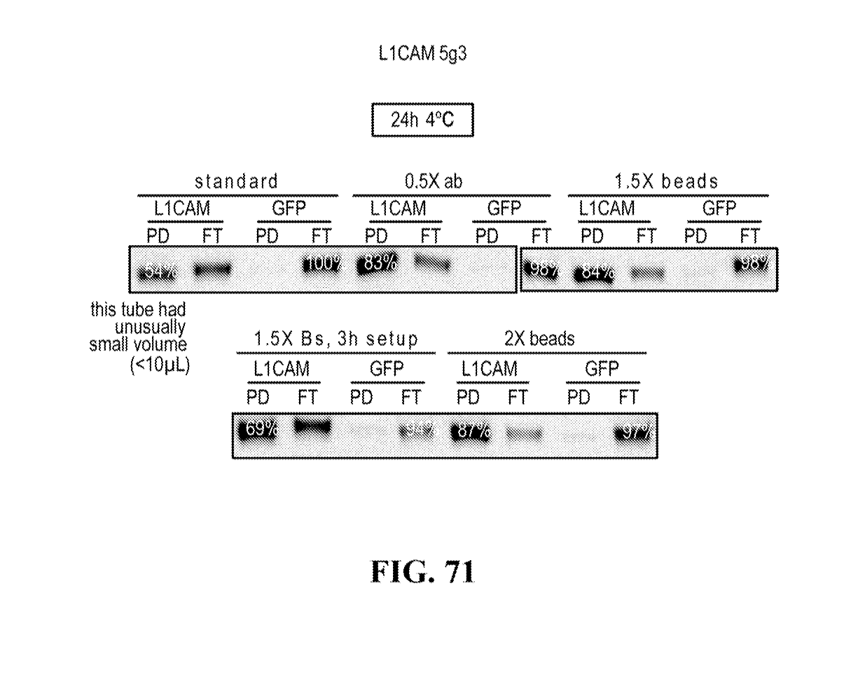

FIG. 11 illustrates exosomes which were isolated from human CSF and mRNA for four genes (detected by qRT-PCR.) Cell RNA is used as a comparison. Two methods of isolating exosomes from CSF were demonstrated: one by running through 0.22 micron filter pelleting at 120,000 g for 2 hours (CSF pellet) and one by extracting RNA directly from CSF after running through 0.22 micron filter without pellet. Similar results were observed by both methods.

### Example 12

### Additional Examples

Mass spectrometry of exosomes from iPS cells and iPS-derived neurons is conducted to find neurons specific membrane proteins found on exosomes. These markers are verified by western blots in iPS and neurons exosomes.

RNA-Seq of exosomes from K562 cells are isolated using CD81 or CD63 antibody-conjugated magnetic beads. The RNA-Seq profiles of exosome subpopulation are compared to the RNA profiles of total exosomes.

RNA-Seq of mRNA from both cells and exosomes from iPS cells and iPS-derived neurons.

RNA-Seq of exosomes from iPS and neuron exosomes isolated using antibody-conjugated magnetic beads to enrich for exosomes expressing the cell type specific proteins. The RNA-Seq profiles of these exosome subpopulations are compared to the RNA profiles of total exosomes from each cell type.

In vitro proof of principle by mixing experiments where Applicants mix cell culture media from iPS cells and neurons and isolate exosomes from the mixed media. Applicants isolate exosomes from the original cell type using antibody-conjugated magnetic beads using the cell type specific markers. Applicants isolate RNA from these exosome subpopulations and perform RNA-Seq to confirm reconstruction of the transcriptome of the original cell type (iPS cells or neurons).

Applicants isolate exosomes from human cerebrospinal fluid (CSF) and perform RNA-Seq.

Applicants enrich for neuron specific exosomes in CSF using antibody-conjugated magnetic beads or a microfluidic device with immobilized antibodies. Applicants then sequence the RNA from these neuron-derived exosomes and to observe enriched expression of neuron-specific genes relative to total CSF exosomes.

### Example 13

### Exosome Pulldown—Detailed Protocol for L1CAM pulldown (PD)

The below example relates to a L1CAM pulldown, but this protocol can also be applied to other cell type-specific or cell-subtype-specific membrane marker(s) present on the surface of the exosomes to be isolated.

After this protocol, one can perform RNA extraction and RNA-Seq, or other single cell type RNA-Seq methods such as SMART-Seq2.

This protocol is used to isolate neuron-specific exosomes from cerebrospinal fluid from a patient.

### Materials:

exosomes, fresh or frozen
Goat anti-mouse IgG beads (500 μL/pulldown) stored in 4° C. fridge (Dynabeads Goat Anti-Mouse IgG, ThermoFisher, Cat. No. 11033)
Isolation Buffer (PBS pH 7.4 supplemented with 1 mg/mL BSA and filtered through 0.22 μm filter) stored in 4° C. fridge
2 mL protein low-bind tubes
primary mouse anti-human antibody for pulldown (10 μL/pulldown) stored at 4° C. or at −20° C. (for L1CAM, Clone 5G3 from BD, Cat. No 554273)
primary rabbit anti-human antibody for Western Blot (for L1CAM, Abcam Cat. EPR18998)

### Bead Setup (Day 1)

1. resuspend vial of goat anti-mouse IgG beads by vortexing gently for 30 s and inverting. visually inspect bottom of vial to make sure there are no clumped beads remaining.
   1. note 1: do not shake vigorously as this will create bubbles.
   2. note 2: if finishing a vial, wash beads down sides using the supernatant of another vial and add them to the second vial.
2. transfer 500 μL (˜2×10⁸) beads into one 2 mL protein low-bind tube per condition.
3. wash the magnetic beads with 1 mL of isolation buffer and mix well by vortexing gently
   1. note: gently=no higher than setting 4.
4. place tubes on the magnet for 1 minute, then remove and discard supernatant.
5. remove tubes from magnet and add 240 μL isolation buffer to beads so they don't dry out.
   1. note: in general, leave beads dry as little as possible.
6. to each tube, add 10 μg of the appropriate antibody clone (for 5g3 from BD, 10 μg=20 μL)
7. incubate on rotating rack in cold room until pulldown, ideally overnight. end over end rotation is fine.

### Pulldown (Day 2 or Later):

1. thaw desired number and type of exosome pellets. for each, record date and volume.
   1. date isolated: ______
   2. μL/pellet: ______
2. retrieve bead tubes from cold room and spin down briefly.
3. place on magnet for 1 minute then remove and discard supernatant.
4. add 1 mL isolation buffer to beads and pipet or vortex to mix.
5. place on magnet for 1 minute and remove supernatant.
6. repeat wash with a second 1 mL isolation buffer.
   1. note: when resuspending beads be sure to visually inspect tube bottom (inverting if necessary) to make sure the bead pellet is gone.
7. after second wash take beads off magnet and add 300 μL isolation buffer to each.
8. add exosomes to beads in desired ratios.
   1. note: if doing mixing experiment, pre-mix the iPS and neuron exosomes in a separate tube as a “mastermix”, using n+1 portions of each for n pulldown conditions. this way the exosomes will be pre-mixed when they hit the beads and the extra pellets remaining in the mix tube will serve as the untreated control to see the exact mixing proportion.
9. add isolation buffer to each bead tube up to 500 μL total volume.
10. place all tubes, bead tubes and control pellets, on rotating rack in cold room (end over end rotation is fine) and note the time.
   After 24 h have Passed:
1. if recovering flow-throughs (FT), prepare an ultracentrifuge tube for each pulldown condition.
2. if extracting protein for western blot, prepare mastermix of 1× sample buffer with 50 mM DTT (e.g. 100 μL 4× Bolt LDS sample buffer, 280 μL water, 20 μL 1M DTT). this must be prepared fresh each day and kept on ice when not in use.
3. collect all tubes from rack in cold room.
4. centrifuge bead tubes briefly to collect samples, then place on magnet for one minute.
5. retrieve isolation buffer from 4° fridge.
6. collect supernatants (500 μL each) and add to respective ultracentrifuge tubes if saving flow-throughs, otherwise discard.
   1. note: do tubes one at a time. immediately after collecting the first supernatant, add 500 μL isolation buffer to wash beads and pipet or vortex gently to mix, placing back on magnet before retrieving the next supernatant.
7. if collecting flow-throughs, add washes to respective UC tubes, pooling with flow-throughs, otherwise discard.
   1. note: if not collecting flow-throughs or extracting protein, the pulldown is now finished: proceed with RNA extraction from beads.
8. if extracting protein, after wash is removed from beads, add 60 μL of the 1× sample buffer+DTT mastermix directly to beads in each tube.
9. to control pellets, add 4× sample buffer to 1× (i.e. if pellet volume is 100 μL add 33 μL) and 1M DTT to 50 mM (i.e. 7 μL into 133 μL)
10. Vortex these at full speed 10 seconds, then place on 70° heating block for 10 minutes.

after 10 minutes place on ice.
11. during 10 minutes, add 34 mL PBS to each ultracentrifuge tube, then balance and spin them for 3 h at 120 000 g.

### During Ultracentrifuge Spin:

1. cool microcentrifuge to 4° C.
2. prepare 1.5 mL protein low-bind tubes and label them the same as pulldown samples.
3. place original bead tubes (currently on ice with eluted protein in sample buffer) on magnet for 1 minute.
4. remove sample buffer with protein (should be ˜60 μL) and move to new 1.5 mL tubes
5. retrieve acetone from −20° freezer and add 4 volumes (˜300 μL) acetone to each tube.
   1. note: be careful not to drip any acetone on tube labels as it will erase them completely.
6. place tubes in ice for 15 minutes, then spin 10 minutes at 12 000 g in 4° C. centrifuge.
   1. note: orient the tubes in the same way around the rotor (i.e. with the cap hinge facing outward) so you know where the pellet will be later (though it should be easily visible).
7. after spin, carefully remove supernatant (again being careful not to drip acetone) with 1 P1000 tip per tube, set to 500 μL volume. leave caps open afterward.
   1. note: while removing supernatant either wait for residual acetone to drip down sides before picking it up or go to all tubes a second time with the P20 to get the last few μL.
8. let pellets air dry for ˜5 minutes, then resuspend all in 20 μL (use P20) 1× sample buffer+DTT. vortex at full speed 10 seconds both before AND AFTER boiling 10 minutes at 70°. visually inspect to ensure pellet is dissolved. store on ice afterward once more.
9. prepare two more 1.5 mL protein low-bind tubes and label them for sample flow-throughs.
10. prepare mastermix of 2× sample buffer with 50 mM DTT (e.g. 100 μL 4× sample buffer, 80 μL water, 20 μL 1M DTT).

### When Ultracentrifuge Spin Finishes,

1. aspirate supernatant from pellets.
2. add 30 μL 2× sample buffer to pellets. vortex each tube at full speed 10 seconds, then leave for 5 minutes
3. pipet up and down to resuspend (i like to use a P200 set to 30 μL) and transfer to 1.5 mL tubes. boil 10 min at 70° C. then put on ice.
4. retrieve acetone from −20° freezer and add 4 volumes (300 μL to be safe) acetone to each tube as before.
5. leave tubes in ice for 15 minutes, then spin 10 minutes at 12 000 g in 4° C. centrifuge.
6. remove acetone from pellets.
7. air dry 5 minutes, then resuspend in 204, 1× sample buffer+DTT.
8. vortex vigorously, boil 10 min at 70° C., vortex again then put on ice.
9. use all protein samples immediately or store at −20° C.

### Western Blotting Parameters:

use 15-well 4-12% bis-tris gel and 1× MES running buffer
   load so wells are in the inner compartment of gel tank
   don't forget to take off tape and blow out wells
load 6 μL seeblue and 0.66 μL magicmark as ladders. use P20 set to 20 μL to load samples.
run ˜30 min at 200V
   make milk buffer while running!
transfer at least 8 min and cut at 62 or 98 kda seeblue bands (L1CAM on top, GJA1 on bottom)
block 30-60 min in 5% milk buffer in PBS+0.1% tween
primaries overnight (1:500 L1CAM rabmab98, 1:500 GJA1 ab47441)
3×10 min washes in PBST, 1-4 h 1:2000 dilution anti-rabbit HRP for secondary, 3×10 min washes in PBST, image with HRP substrate spray bottle

### Example 14

### Additional Example for a Pull Down (PD)

In general, the pull down can be performed with the following conditions: 0.1 mL-2 mL of volume at temperatures between 4 C and 37 C, for time periods between 0.5 to 48 hours.

Another example is a pulldown in 24 hours at 4 C in 0.5 mL volume.

### Example 15

### Additional Example for Extracellular Vesicle (EV) Isolation and Analysis by Western Blotting

This example provides a detailed protocol for isolating EVs by differential ultracentrifugation and analyzing EV proteins (such as the tetraspanins CD9, CD63, and CD81) by western blotting.

Materials

Store all materials at room temperature unless otherwise stated.

### 1. Cell Culture

1. Cells and cultureware
2. Fetal Bovine Serum (FBS)-depleted media or defined media without FBS (see Note 1)

### 2. EV Isolation

1. PBS without Ca++/Mg++
2. HEPES buffer (optional) (see Note 2)
3. 50 mL Falcon tubes (Fisher Scientific)
4. 0.22 μm Steriflip filter tubes (Fisher Scientific)
5. Ultracentrifuge and rotor
6. Polyallomer ultracentrifuge tubes (Beckman Coulter)

### 3. Western Blot

1. Sample Buffer: Bolt 4× LDS Sample Buffer (Thermo Fisher Scientific). Store at 4° C.
2. Bolt 10× Reducing Buffer (optional) (Thermo Fisher Scientific) (see Note 3)
3. RIPA buffer (optional) (see Note 4)
4. A660 or BCA protein quantification assay (optional) (see Note 5)
5. Running buffer: 100 mL 20× MES SDS running buffer (Thermo Fisher Scientific), 1900 mL deionized water.
6. 4-12% Bis-Tris Gels. Store at 4° C.
7. Gel tank (XCell SureLock® Mini, Thermo Fisher Scientific) and electrophoresis equipment.
8. MagicMark XP Western protein standard (Thermo Fisher Scientific). Store at −20° C.
9. SeeBluePlus2 protein ladder (Thermo Fisher Scientific). Store at 4° C.
10. XCell II Blot Module and sponges (Thermo Fisher Scientific) (see Note 6).
11. Methanol
12. Transfer buffer: 100 mL Bolt 20× transfer buffer (Thermo Fisher Scientific), 400 mL methanol, 1500 mL deionized water
13. PVDF or nitrocellulose membranes.
14. Milk powder.
15. Tween-20.
16. PBST: PBS with 0.1% vol/vol Tween-20. Store at 4° C.
17. Cold room with a tilting rocker (not orbital)
18. Plastic containers to hold membranes, such as PerfectWestern™ containers.
19. Flat tweezers for handling membranes.
20. Antibodies to proteins of interest
21. HRP-conjugated secondary antibody for visualization
22. HRP substrate, such as SpectraQuant™-HRP CL Chemiluminescent detection reagent (BridgePath Scientific)

Methods

### 1. EV Isolation

1. Culture cells under standard conditions to 50-70% confluency.

### Day 1

### For Suspension Cells:

1. Spin down desired total number of cells (see Note 7) in 6 50 mL Falcon tubes at 300×g for 5 minutes.
2. Aspirate media and resuspend each cell pellet in 40 mL FBS-depleted or defined media without FBS (see Note 1). Transfer contents of each Falcon tube to T75 flask and return to incubator.

### For Adherent Cells:

1. Aspirate media from 12 15 cm plates.
2. Add 20 mL FBS-depleted or defined media without FBS per plate (see Note 1). Return cells to incubator.

### Day 2

1. After 24 hours, take off all media and divide among 50 mL falcon tubes.
2. Spin at 300×g for 10 minutes at RT (to pellet the cells).
3. Transfer supernatant to new 50 mL tubes leaving cell pellet behind. If cell protein is to be analyzed alongside EVs, one cell pellet can at this step be resuspended in the desired lysis buffer (see Note 4, Note 5).
4. Spin at 2000×g for 10 minutes at RT (to pellet any dead cells).
5. Transfer supernatant to new 50 mL tubes leaving cell pellet behind.
6. Spin supernatant at 16,500×g for 20 minutes at 4° C. (to pellet large EVs).
7. Transfer supernatant to new 50 mL tubes, leaving pellet behind.
8. Pass supernatant through Steriflip 0.22 μm filter.
9. Transfer supernatant to polyallomer ultracentrifuge tubes. Centrifuge at 120,000×g (26,500 RPM with SW32Ti rotor) for 70 minutes at 4° C.
10. Remove most of supernatant, leaving ˜2 cm of media above pellet. Add 5 mL PBS to each tube. Vortex on medium speed for a few seconds. Fill to top of each tube with PBS.
11. Again, centrifuge at 120,000×g for 70 minutes at 4 degrees.
12. Aspirate all of supernatant with Pasteur pipet without touching bottom of tube where pellet is located (see Note 8).
13. Resuspend pellet either in PBS or directly in the desired lysis buffer for Western blot (see Note 4, Note 5).

### 2. Western Blot

14. Add 100 uL 1× Sample Buffer to each pellet, or add 4× sample buffer to a final concentration of 1× (i.e. add 25 μL 4× sample buffer to 75 μL sample) in a pre-isolated EV sample. Vortex on high speed to mix. If in ultracentrifuge tubes pipet up and down to further disrupt pellet, then transfer to Eppendorf tubes.
15. Incubate at 70° C. for 10 minutes.
16. Make 2 L of 1× MES SDS running buffer (100 mL buffer into 1900 mL deionized water)
17. Make 5% milk buffer: 2.5 g dried milk into 50 mLs PBST (PBS+0.1% v/v Tween-20). Tumble at 4° for an hour. (see Note 9)
18. Prepare 4-12% bis-tris gel in gel tank. Add 1× MES running buffer to top of gel. Don't forget to rinse wells.
19. Load wells. For ladder use 34, MagicMark+6 μL SeeBluePlus2, in separate lanes if possible (see Note 10).
20. Run 40 minutes at 150 V, 22 minutes at 200 V, or until blue dye reaches gel foot.
21. While gel is running, prepare for transfer (see XCell surelock manufacturer instructions [6] for more detail)
22. Make 2 L transfer buffer (100 mL Bolt 20× transfer buffer, 400 mL methanol, 1500 mL water)
23. If using PVDF membranes, place one membrane in empty tip box lid with a few mL methanol to activate it. Rinse several times with transfer buffer, dumping excess into large sandwich making tray, then rock gently in hand for several minutes. For nitrocellulose membranes, simply soak in transfer buffer for a minute.
24. Soak sponges in transfer buffer, squeezing out bubbles as much as possible (see Note 11). Briefly immerse filter papers in transfer buffer as well.
25. Build sandwich up from the bottom in the following order: anode, sponges, filter paper, gel, transfer membrane, filter paper, sponges (see FIG. 1, Note 12).
26. When sandwich is ready for gel, take gel out of tank and rinse it off. Crack open plastic casing. Cut off wells and foot so that remainder is completely flat and lay carefully on filter paper.
27. Squeeze sandwich together in holder and insert into gel tank. If reusing the same tank make sure to pour out gel running buffer and rinse with deionized water.
28. Use fresh transfer buffer to fill in sandwich from the top. Open and close clamp several times to let the buffer soak down through.
29. Fill the rest of the gel box with deionized water, which will serve as a heat sink.
30. Put on lid and run 1.5-2 hours at 30 V, tapping firmly on occasion to remove bubbles (see Note 11).
31. When done, turn off current, pull out sandwich in holder, put it back in large tray (minus transfer buffer) then unpack it carefully. Peel away filter paper very slowly to check for protein transfer (see Note 13).
32. As soon as you peel off membrane, take a blade and cut off upper right hand corner to mark “top” face (face which was touching gel).
33. Place membrane in PerfectWestern box containing 5-10 mL milk buffer (as much as necessary to cover membrane completely). If using PVDF membranes, ensure that the membrane does not dry out at any step.
34. To block, place membrane in milk buffer on rocker in the cold room and let rock at least half an hour. Conduct all further steps in the cold room if possible.
35. After at least half an hour, pour off block and add 10 mL primary antibody diluted 1:1000 in milk buffer (see Note 14). Leave overnight rocking in the cold room

### Day 3

36. Pour off primary, take PBST, pour in, swish, pour off, 2×, then do 3 washes in PBST of ˜10 minutes each, rocking in the cold room
37. Add 10 mL secondary antibody diluted 1:2000 in milk buffer (see Note 14). Leave rocking in the cold room for at least one hour.
38. Pour off secondary, take PBST, pour in, swish, pour off, 2×, then do 3 washes in PBST of ˜10 minutes each, rocking in the cold room
39. Bring the membrane (in fresh PBST), equal volumes of each component of HRP substrate (reagent A and B; see 2.2.21) and an empty falcon tube to the imaging stage.
40. Mix reagent A and B together immediately before use. Pour PBST off membrane and pour A/B mix on. Let sit for a minute then image, using tweezers to handle membrane. (see Note 15).

### Notes

1. Since fetal bovine serum contains bovine EVs, it is important for downstream analysis that media from which EVs will be isolated is either FBS-free or has been depleted of vesicles by overnight ultracentrifugation at 120,000×g. A convenient formulation is to make media with 2× FBS and ultracentrifuge it overnight, then remove and keep the supernatant, diluting it 1:1 in the base media to bring it to 1×. Some cells will still not like this media and so we advise collecting EVs for 24 hours.
2. For storage of EVs at −80° C. we recommend the addition of HEPES buffer to a final concentration of 20 mM to stabilize pH over freeze-thaw cycles (to PBS or other buffers).
3. Protein gel electrophoresis can be either denaturing or non-denaturing (“native”, i.e. retaining the original folded structure) and either reducing (where Cys-Cys disulfide bonds are specifically broken) or non-reducing. Though reducing can help to solubilize a concentrated or complex sample, tetraspanins such as CD63, CD81 and CD9 require non-reducing electrophoresis for western blotting, as the epitope recognized by antibodies to these proteins usually relies on several disulfide bonds to fold properly and be recognized.
4. Transmembrane proteins, particularly those with four or more membrane-spanning regions, can be difficult to extract from lysates. We have had success extracting tetraspanins with LDS sample buffer alone (2.3.1) but other proteins may require some optimization of lysis buffer for efficient extraction. RIPA buffer is one of the harsher common buffers and is well suited for this purpose. When extracting membrane proteins from cells, it is often helpful to centrifuge the lysate at high speed (>12,000×g) for 10 minutes and take the supernatant, leaving behind the membrane and insoluble material which can interfere with electrophoresis.
5. Many common protein quantification assays (such as A660 and BCA) rely on a colorimetric readout, and are thus incompatible with the bromophenol blue-containing LDS sample buffer. This protocol does not explicitly describe how to quantify protein in a lysate, but note that if you do wish to quantify the protein in your samples, you should lyse the cells or vesicles in RIPA (2.3.2) or another clear buffer, quantify, and then add 4× LDS sample buffer to 1× concentration prior to immunoblotting.
6. Materials 2.3.10 through 2.3.13 are required for a traditional wet transfer of proteins to a membrane. These can be substituted with other materials of your choice for dry or semi-dry transfer. For example, we have found the iBlot dry blotting system from Thermo Fisher is convenient and effective, though not all labs may have the required equipment.
7. The total number of cells per isolation should be determined by the total volume of media from which you are able to isolate EVs. The limiting factor will likely be the volume capacity of your ultracentrifuge tubes (e.g. the SW32Ti rotor can hold 6 tubes with a volume of ˜38 mL each, so the max volume per isolation is 228 mL). Start with a few extra mL of media per flask to account for some loss throughout the centrifugation steps and culture the number of cells necessary to achieve 50%-70% confluence in this volume.
8. The pellet at this stage will most likely not be visible. It is possible to remove all but 20-30 μL of the supernatant by tilting the tube to pool the liquid on one side and carefully avoiding touching the center of the tube bottom. We have also found that it is helpful to remove all but ˜2 cm of supernatant and wait 30 seconds before aspirating the final few mLs, as otherwise some liquid clings to the sides of the tube and makes the final residual volume >50 μL.
9. The proteins in the milk buffer associate with proteins in the membrane and block non-specific antibody interactions. There are many formulations of blocking solution available but we have found milk to be cheap and effective. It is important to make this buffer fresh (it should be a few days old at most and stored at 4° with rotation).
10. MagicMark XP is a protein standard ladder containing IgG binding sites (you will see it on the final western blot, not in the gel) while SeeBlue is a pre-stained protein standard ladder which you should see in the gel and membrane but not in the final blot. These can be mixed if necessary but will run better in separate wells. SeeBlue is useful for evaluating how far the gel has run and if the transfer was successful (see Note 13) as well as for horizontally cutting the membrane in order to blot for proteins of different molecular weights, e.g. CD63 and CD81.
11. Air bubbles anywhere in the sandwich can prevent successful transfer of proteins to the membrane in that spot, so it's important to squeeze the sandwich tightly and firmly tap the XCell mini tank periodically (as many times as is convenient) while transfer occurs.
12. Use as many sponges as necessary to form a tight sandwich. Generally at least three sponges on either side of the gel and membrane (six total) will suffice, but the tighter the better.
13. Carefully peel away the top corner of the membrane closest to where the SeeBlue ladder was run and check for the location of the colored bands. If the transfer worked, some or all of them should now be on the membrane instead of the gel. Specifically, check that the SeeBlue bands in the molecular weight range of your protein of interest (for example, the 28 kda band is close to the size of CD81) are on the membrane. If they are still on the gel, you can carefully reconstruct the sandwich (ensure that the gel and membrane do not shift relative to one another) and run it slightly longer. Keep in mind that running the transfer for too long will cause the lower molecular weight bands to pass through the membrane onto the filter paper, at which point they cannot be recovered.
14. As different antibodies have different affinities for their targets, it is often necessary to experimentally determine the optimal antibody dilutions for immunoblotting. Generally these fall within 1:100 and 1:5000 and are lower (i.e. more dilute) for the secondary antibody. We recommend starting with a higher dilution (more concentrated) to ensure a strong signal and diluting further as necessary to eliminate background or conserve reagents.
   If using Image Lab software to visualize blot, can set to “signal accumulation mode” to determine optimal exposure.

### Example 15

### Further Example for an Exosome Pulldown Protocol

Materials
exosomes, fresh or frozen
goat anti-mouse IgG beads (500 μL/pulldown) stored in 4° C. fridge
Isolation Buffer (PBS pH 7.4 supplemented with 1 mg/mL BSA and filtered through 0.22 μm filter) stored in 4° C. fridge
2 mL protein low-bind tubes
primary antibody (10 μg/pulldown) stored at 4° C. or at −20°
Buffer RLT (Qiagen)
Dynabeads MyOne Silane Beads (Thermo Fisher Scientific)
4× Bolt LDS Sample Buffer (Thermo Fisher Scientific)
Ethanol
Acetone

Bead Setup (Day 1)
1. The day before exosome isolation, set up beads. Resuspend vial of goat anti-mouse IgG beads by vortexing gently for 30 s and inverting.
2. In the case of CD81, transfer 250 μL (˜1×10⁸) beads into one 2 mL protein low-bind tube per condition. In the case of L1CAM, use 500 uL beads.
3. wash the magnetic beads with 1 mL of isolation buffer and mix well by vortexing gently (vortex setting<4)
4. place tubes on the magnet for 1 minute, then remove and discard supernatant.
5. remove tubes from magnet and add 230 μL isolation buffer to beads so they don't dry out.
6. to each tube, add 10 μg of the appropriate antibody clone (for CD81 antibody, 10 μg=20 μL)
7. incubate on rotating rack in cold room until pulldown, overnight.

Perform Exosome Isolation (day 2). After Second Ultracentrifuge Spin:
1. After aspirating supernatant from exosome ultracentrifuge tube, add 200 uL of PBS to exosome pellet.
2. retrieve bead tubes from cold room and spin down briefly.
3. place on magnet for 1 minute then remove and discard supernatant.
4. add 1 mL isolation buffer to beads and pipet or vortex to mix.
5. place on magnet for 1 minute and remove supernatant.
6. repeat wash with a second 1 mL isolation buffer.
7. after second wash, take beads off magnet and add 300 μL isolation buffer to each.
8. add exosomes to tube of beads (final volume is now 500 uL).
9. Bind exosomes to antibody-coated beads. In the case of CD81, place on rotating rack at 37 C for 1 hour. For L1CAM, bind at 4 C for 24 hours.

After Bead Exosome Incubation:
1. collect all tubes from rotating rack in 37 C.
2. centrifuge bead tubes briefly to collect samples, then place on magnet for one minute.
3. retrieve isolation buffer from 4° fridge.
4. collect supernatants (500 μL each) from beads and discard.
5. Wash each tube of beads with 500 uL isolation buffer.
6. Remove and discard isolation buffer.
7. Add 70 uL RLT to exsome bound beads and proceed with RNA extraction.
8. Vortex beads with RLT buffer and leave on ice for 1 minute.
9. Prepare silane beads by moving 5 uL to a well of a 96 well plate and then washing beads in 100 uL RLT.
10. Resuspend silane beads in 10 uL RLT.
11. Put tubes with exosomes in RLT back on magnet and wait 1 minute. Then take exosome lysate in RT and transfer to wells of 96 plate.
12. Add silane beads to exosome lysate.
13. Add 120 uL 100% ethanol to each well and pipet up and down 10 times. Incubate for 5 minutes to let RNA bind to Silane beads.
14. Put plate on magnet and wait 2 minutes.
15. Take off supernatant (consisting of RLT buffer and ethanol) and transfer to Lo-bind eppendorf tubes for protein extraction. Keep on ice.
16. Wash silane beads with 150 uL 70% ethanol.
17. Take off supernatant and repeat wash.
18. Take off supernatant and allow beads to dry, about 10 minutes.
19. Resuspend beads in water and prepare SMART-Seq2 Reverse Transcription mix. Transfer RNA form silane beads to RT mix.
20. While RNA is undergoing reverse transcription, do acetone precipitation of protein. Add 4 volumes of ice cold acetone to each tube (800 uL). Vortex and incubate at −20 C for 30 minutes.
21. Centrifuge for 10 minutes at 4 C at maximum 16000 RCF.
22. Discard supernatant and wash with 200 uL ice cold 100% ethanol. Centrifuge for 10 minutes at 4 C at maximum 16000 RCF.
23. Discard supernatant and air dry until pellet is dry, about 10 minutes. Resuspend in 75 uL water. Add 25 uL 4× Bolt LDS Sample Buffer and pipet up and down. Freeze protein in −20 C.

### Example 16

### RNA Extraction for Low Input Exosome Samples: Extraction with Silane Magnetic Beads and Subsequent RNA Analysis

RNA extraction for low input exosome samples was performed using magnetic nucleic acid binding beads (silica Dynabeads functionalized with silane) as described at Example 15.

qRT-PCR wasperformed using purified cell RNA and exosome RNA from K562 cells to evaluate extraction rate/sample loss during RNA purification.

The samples in qRT-PCR experiment were as follows:

K562 Cell 1 RNA

K562 Cell 2 RNA

K562 Total Exosomes 1

K562 Total Exosomes 2

CD83 Pulldown 1

CD83 Pulldown 2

10 pg RNA Silane Dnase treated

100 pg RNA Silane Dnase treated

10 pg RNA Silane ×3 Dnase treated

100 pg RNA Silane ×3 Dnase treated

10 pg RNA no DNase Silane after

100 pg RNA no DNase Silane after

10 pg RNA no Silane

100 pg RNA no Silane

10 pg no RT control no Silane

100 pg no RT control no Silane

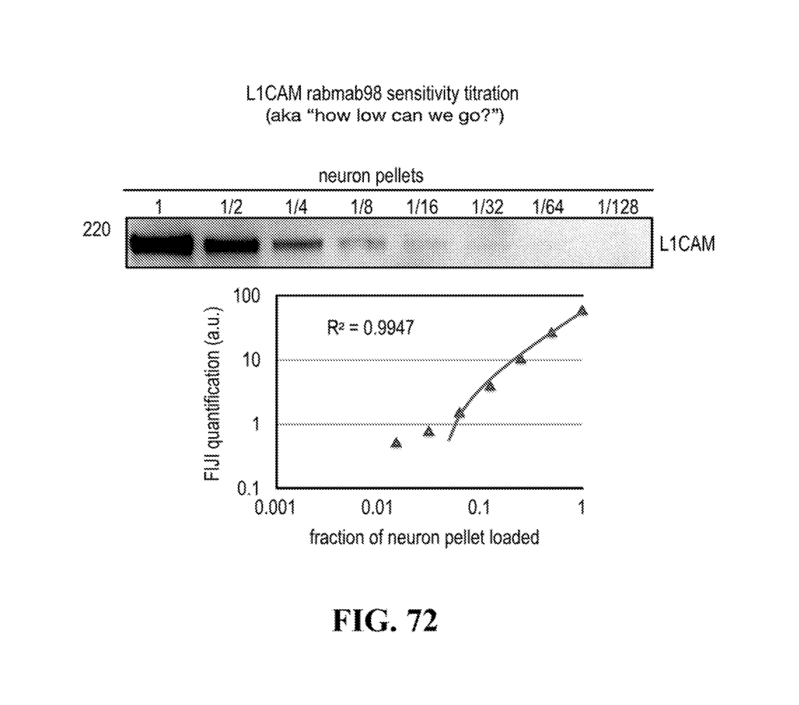

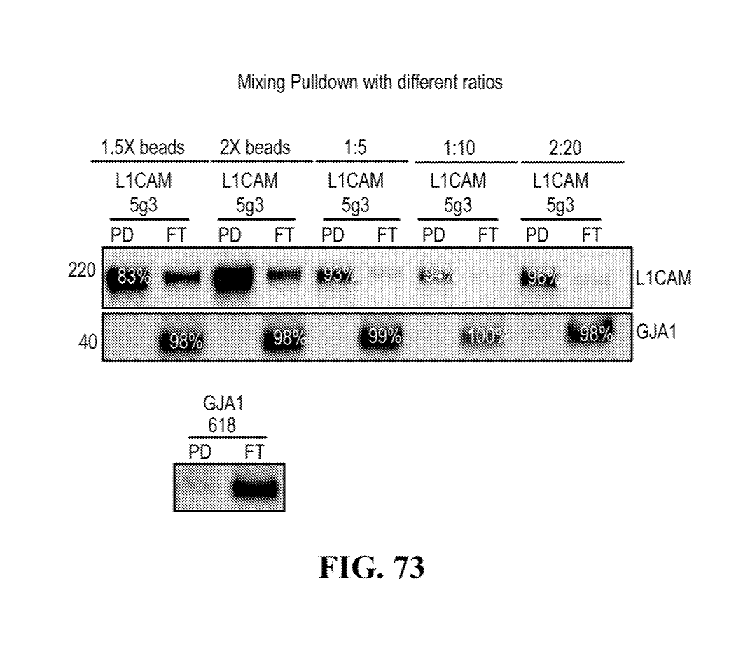

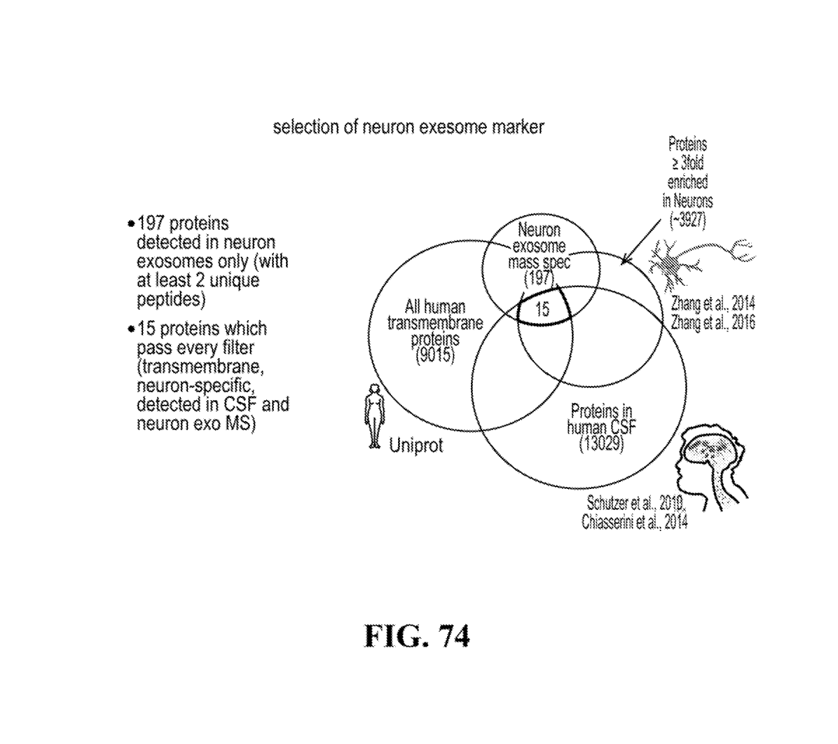

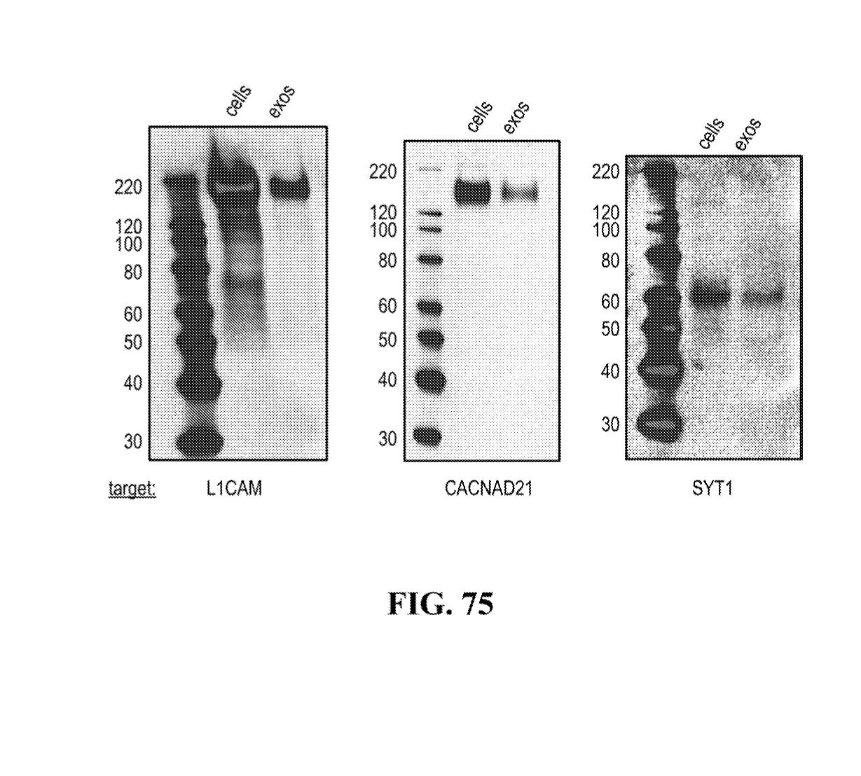

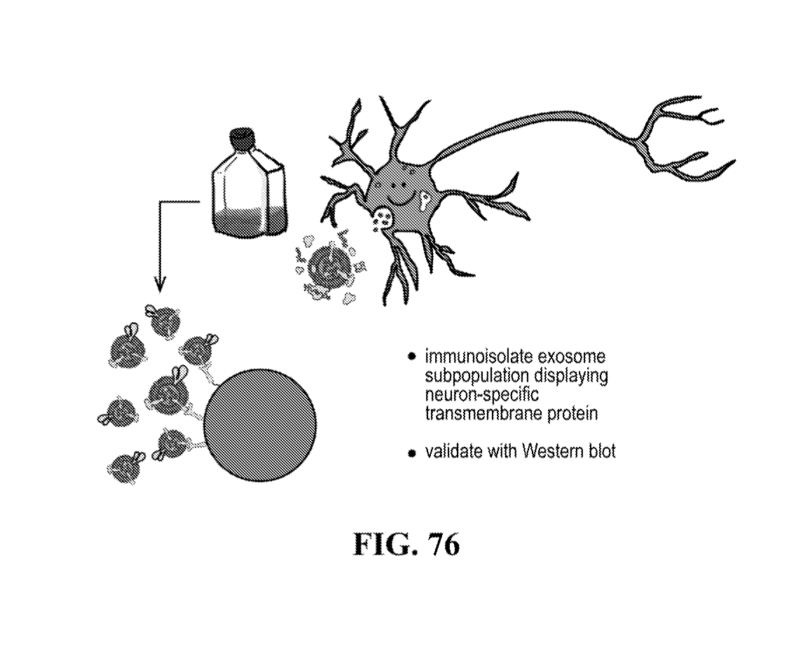

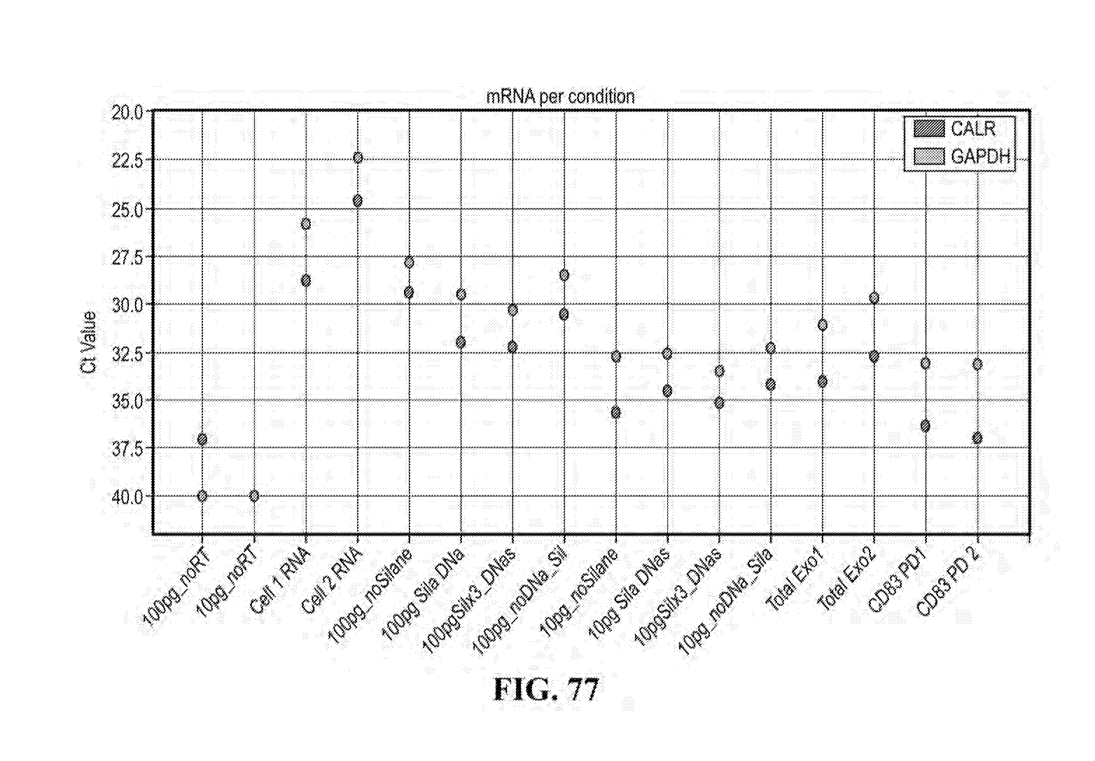

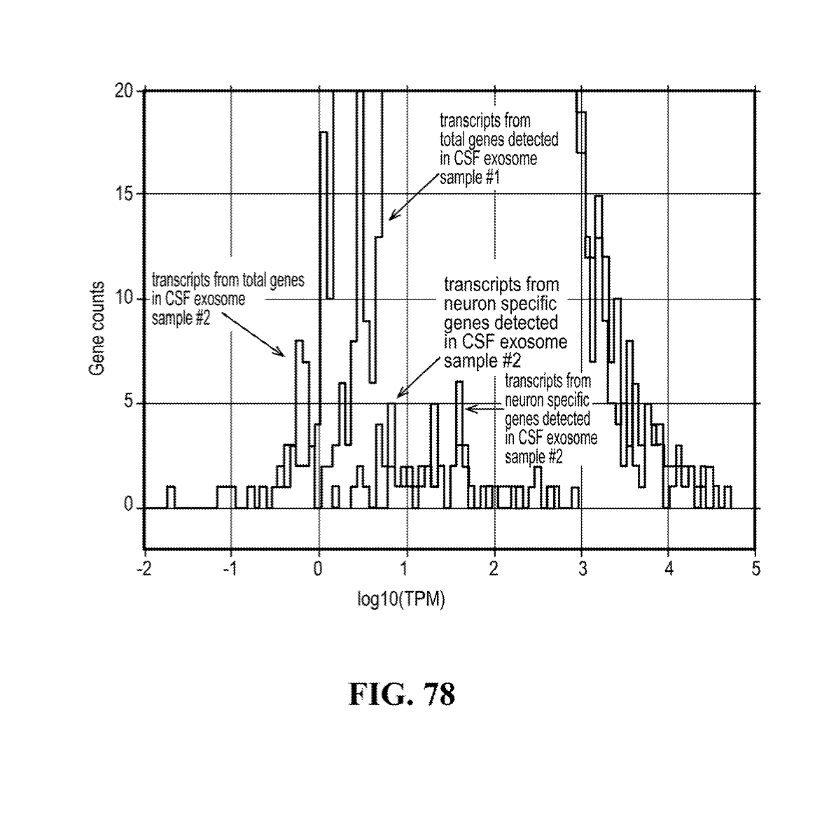

10 pg or 100 pg of purified RNA from K562 cells were used alongside three samples: RNA from K562 cells, RNA from K562 exosomes and RNA from a CD83 pulldown (followed by the optimized RNA extraction protocol). Then, qRT-PCR was performed for two mRNAs to quantify the relative amounts of RNA. The results are shown at FIG. 76.

### Example 17

### Isolation of Neuron-Specific Exosomes, Followed by RNA Extraction and Analysis

Neuron specific exosomes were isolated from CSF and their RNA was sequenced.

Two samples of CSF (300 uL each) were provided, their exosomes were isolated, and RNA-Seq was performed on them using SMART-Seq2.

The inventors were able to detect neuron specific transcripts in CSF exosomes, which indicates the presence of neuronal derived exosomes.

The results are shown at Figure **Slide neuronal genes in CSF RNA seq* showing the neuronal genes that could be deteted in CSF using RNA seq. Total exosomes from CSF were isolated and transcripts from neuron-specific genes are detected:

Green: transcripts from neuron specific genes detected in CSF exosome sample #1

Pink: transcripts from total genes in CSF exosome sample #1

Blue: transcripts from neuron specific genes detected in CSF exosome sample #2

Yellow: transcripts from total genes in CSF exosome sample #2

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method for the selection of an antibody for the isolation of cell type-specific exosomes or cell-subtype-specific exosomes from a biological sample, said method comprising:
(a) providing a biological sample comprising exosomes from a cell population,
(b) selecting one or more cell type-specific or cell-subtype-specific membrane marker(s) present on the surface of the exosomes to be isolated,
(c) selecting an antibody against each of the one or more the cell type-specific or cell-subtype-specific membrane marker(s) of step (b), wherein said antibody (resp. each of said antibodies) has (have):
a capture rate of 30% or more for the cell type-specific or cell-subtype-specific membrane marker, and
a specificity of 70% or more for the cell type-specific or cell-subtype-specific membrane marker.

2. The method of claim 1, further comprising
(d) performing immuno-isolation of exosomes from the biological sample of step (a) using the antibody or antibodies of step (c), thereby providing isolated cell type-specific exosomes or cell-subtype-specific exosomes.

3. The method of claim 1, wherein the antibody has a capture rate of 30% or more, 35% or more, 40% or more, 45% or more, 50% or more, 55% or more, 60% or more, 65% or more, 70% or more, 75% or more, 80% or more, 85% or more, or 90% or more, for the cell type-specific or cell-subtype-specific membrane marker.

4. The method of claim 3, wherein the antibody has a specificity of 75% or more, 80% or more, 85% or more, 90% or more, 92% or more, 95% or more, 97% or more, or 99% or more for the cell type-specific or cell-subtype-specific membrane marker.

5. The method of claim 2, wherein step (b) comprises selecting two cell type-specific or cell-subtype-specific membrane markers present on the surface of the exosomes to be isolated, and optionally wherein the immune-isolation of step (d) comprises simultaneous or sequential immune-isolation using the antibodies against respective two cell type-specific or cell-subtype-specific membrane markers present on the surface of the exosomes to be isolated.

6. The method of claim 2, wherein step (b) comprises:
generating or retrieving a list of membrane proteins of said mammal species, and/or
generating or retrieving a list of proteins present or enriched in the cell type or cell subtype of said mammal species, and/or
where the biological sample comprises a body fluid or is derived from a body fluid from a mammal, generating or retrieving a list of proteins present or enriched in the body fluid of said mammal species, and/or
generating or retrieving a list of cell type-specific or cell-subtype-specific membrane exosome proteins of said mammal species,
and wherein step (b) comprises selecting a protein present on two, three or four of these lists.

7. The method of claim 1, wherein step (b) comprises:
generating or retrieving a list of membrane proteins of said mammal species,
generating or retrieving a list of proteins present or enriched in the cell type or cell subtype of said mammal species,
where the biological sample comprises a body fluid or is derived from a body fluid from a mammal, generating or retrieving a list of proteins present or enriched in the body fluid of said mammal species, and
generating or retrieving a list of cell type-specific or cell-subtype-specific membrane exosome proteins of said mammal species,
and wherein step (b) further comprises selecting a protein present on all four of these lists.

8. The method of claim 1, wherein the one or more cell type comprises cells derived from the endoderm, cells derived from the mesoderm, or cells derived from the ectoderm.

9. The method of claim 8, wherein cells derived from the endoderm comprise cells of the respiratory system, the intestine, the liver, the gallbladder, the pancreas, the islets of Langerhans, the thyroid or the hindgut.

10. The method of claim 8, wherein cells derived from the mesoderm comprise osteochondroprogenitor cells, muscle cells, cells from the digestive systems, renal stem cells, cells from the reproductive system, bloods cells or cells from the circulatory system (such as endothelial cells).

11. The method of claim 8, wherein cells derived from the ectoderm, comprise epithelial cells, cells of the anterior pituitary, cells of the peripheral nervous system, cells of the neuroendocrine system, cell of the teethes, cell of the eyes, cells of the central nervous system, cells of the ependymal or cells of the pineal gland.

12. The method of claim 11, wherein cells from the central nervous system and the peripheral nervous system comprise neurons, Schwann cells, satellite glial cells, oligodendrocytes or astrocytes.

13. The method of claim 12, wherein neurons comprise interneurons, pyramidal neurons, gabaergic neurons, dopaminergic neurons, serotoninergic neurons, glutamatergic neurons, motor neurons from the spinal cord, or inhibitory spinal neurons.

14. The method of claim 1, wherein the one or more cell-type is a cancer cell or a circulating tumor cell (CTC), such as cancer cell or CTC derived from any cell-types or cell subtypes derived from the endoderm, cells derived from the mesoderm, or cells derived from the ectoderm.

15. The method of claim 1, wherein the antibody is immobilized on a solid substrate.

16. The method of claim 15, wherein the solid substrate is selected from a purification column, a microfluidic channel or beads, such as magnetic beads.

17. The method of claim 2, wherein the immuno-isolation comprises a microfluidic affinity based isolation, a magnetic based isolation, a pull-down isolation or a fluorescence activated sorting-based isolation.

18. The method of claim 1, wherein the biological sample comprises a body fluid or is derived from a body fluid, wherein the body fluid was obtained from a mammal.

19. The method of claim 18, wherein the body fluid is selected from amniotic fluid, aqueous humor, vitreous humor, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof.

20. The method of claim 1, wherein step (b) comprises:
generating or retrieving a list of membrane proteins of said mammal species,
generating or retrieving a list of proteins present or enriched in a neural tissue cell type or cell subtype of said mammal species,
where the biological sample comprises cerebrospinal fluid or is derived from cerebrospinal fluid from a mammal, generating or retrieving a list of proteins present or enriched in cerebrospinal of said mammal species, and
generating or retrieving a list of cell type-specific or cell-subtype-specific membrane exosome proteins of said mammal species,
and wherein step (b) further comprises selecting a protein present on all of these lists.

21. The method of claim 20, wherein said cell type is selected from neurons, Schwann cells, satellite glial cells, oligodendrocytes or astrocytes.

22. The method of claim 21, wherein said cell type is selected from neurons and wherein said cell subtype is selected from interneurons, pyramidal neurons, gabaergic neurons, dopaminergic neurons, serotoninergic neurons, glutamatergic neurons, motor neurons from the spinal cord, or inhibitory spinal neurons.

23. The method of claim 22, wherein the one or more cell type-specific or cell-subtype-specific membrane marker(s) present on the surface of the exosomes to be isolated comprises L1CAM, CACNA2D1 or SYT1.

24. A method for the preparation of exosomal RNA from a biological sample, said method comprising:
(i) providing a biological sample comprising exosomes from a cell population,
(ii) performing immuno-isolation of exosomes from the biological sample of step (i) using the antibody or antibodies selected according to the method of claim 1,
(iii) extracting RNA from the isolated exosomes of step (ii),

25. The method of claim 24, wherein the exosomal RNA is total exosomal RNA.

26. The method of claim 24, wherein the exosomal RNA comprises exosomal messenger RNA.

27. The method of claim 24, wherein the exosomal RNA is total exosomal messenger RNA.

28. A method for the determination of cellular RNA content in a cell population, said method comprising:
(A) providing a biological sample comprising exosomes from said cell population,
(B) performing immuno-isolation of exosomes from the biological sample of step (A) using the antibody or antibodies selected according to the method of claim 1,
(C) extracting RNA from the isolated exosomes of step (B), so as to provide exosomal RNA,
(D) analyzing the exosomal RNA extracted at step (C),
(E) estimating, as a function of the result from step (D), the cellular RNA content in the cell population.

29. The method of claim 28, wherein step (E) is performed based on a predicted correlation between exosomal RNA content and cellular RNA content.

30. The method of claim 28, wherein said determination comprises a qualitative determination.

31. The method claim 28, wherein said determination comprises a quantitative determination.

32. The method of claim 28, wherein said quantitative determination comprises determination of relative abundance of two RNAs.

33. The method of claim 28, wherein said determination comprises determination of mRNA profiles.

34. The method of claim 28, wherein said RNA comprises messenger RNA (mRNA).

35. The method of claim 28, wherein said RNA comprises micro RNA (miRNA) or long non-coding RNA (lncRNA).

36. The method of claim 28, wherein step (D) comprises a qualitative determination, RNA sequencing (RNA seq), array analysis, reverse transcription polymerase chain reaction (RT-PCR), quantitative reverse transcription polymerase chain reaction (qRT-PCR).

37. The method of claim 28, wherein step (D) comprises analyzing one or more sequence/s of interest.

38. The method of claim 37, comprising testing for the presence or absence of said sequence/s of interest, analyzing for one or more allelic variants of a sequence of interest, testing for presence or absence of said allelic variants.

39. The method of claim 28, wherein step (D) comprises genome-wide analysis.

40. The method of claim 28, wherein step (D) comprises transcriptome profiling.

41. The method of claim 28, wherein the determination is time-lapse.

42. The method of claim 28, for use in diagnosis, prognosis, or a screening process.

43. (canceled)

44. (canceled)

45. The method of claim 28, wherein the method determines the cellular RNA content of a single cell type or of a single cell subtype.

46. A method for the diagnostic or prognostic of a disorder of interest in a subject, comprising:
(I) selecting a biomarker, wherein said biomarker is associated with said disorder and wherein said biomarker may be determined in a cell type that is found in the subject to be in contact with a body fluid,
(II) providing a biological sample from said body fluid from said subject,
(III) estimating the cellular RNA content of said biomarker in the biological sample of step (II) by performing the method of claim 28.

47. The method of claim 46, wherein the cellular RNA content is the cellular content of a single cell type or of a single cell subtype.

48. The method of claim 46, further comprising (IV) determining, from the results of step (III), the status of the biomarker selected at step (I).

49. The method of claim 46, wherein the biomarker is selected from expression of a given open reading frame (ORF), overexpression of a given open reading frame (ORF), repression of a given open reading frame (ORF), over-repression of a given open reading frame (ORF), expression of a given allelic variant, relative level of expression of a given open reading frame (ORF), presence of a mutation in a given open reading frame (ORF).

50. The method of claim 46, wherein said disorder is a blood disorder and said biomarker is a biomarker that may be determined in one or more cell type/s that is/are found in the subject to be in contact with blood.

51. The method of claim 46, wherein said disorder is a brain or spine disorder and said biomarker is a biomarker that may be determined in one or more cell type/s that is/are found in the subject to be in contact with cerebrospinal fluid.

52. The method of claim 46, wherein said disorder is a heart disorder and said biomarker is a biomarker that may be determined in one or more cell type/s that is/are found in the subject to be in contact with blood or pericardial fluid.

53. The method of claim 46, wherein said disorder is a prostate or bladder disorder and said biomarker is a biomarker that may be determined in one or more cell type/s that is/are found in the subject to be in contact with urine.

54. The method of claim 46, wherein said disorder is an eye disorder and said biomarker is a biomarker that may be determined in one or more cell type/s that is/are found in the subject to be in contact with tears.

55. The method of claim 46, wherein said disorder is a lung disorder and said biomarker is a biomarker that may be determined in one or more cell type/s that is/are found in the subject to be in contact with pleural fluid.


SEQUENCE LISTING (8 sequences)

SEQ ID NO: 1 - DNA, 20 bp, Artificial Sequence
  Synthetic primer
gggtactgtggagggctttg

SEQ ID NO: 2 - DNA, 24 bp, Artificial Sequence
  Synthetic primer
aggaggtttgaataagccatctga

SEQ ID NO: 3 - DNA, 19 bp, Artficiail Sequence
gtatgcctgccgtgtgaac

SEQ ID NO: 4 - DNA, 22 bp, Artificial Sequence
  Synthetic primer
aaagcaagcaagcagaatttgg

SEQ ID NO: 5 - DNA, 18 bp, Artificial Sequence
  Synthetic primer
cggcatcgtcaccaactg

SEQ ID NO: 6 - DNA, 23 bp, Artificial Sequence
  Synthetic primer
aacatgatctgggtcatcttctc

SEQ ID NO: 7 - DNA, 23 bp, Artificial Sequence
  Synthetic primer
ggtggtctcctctgacttcaaca

SEQ ID NO: 8 - DNA, 23 bp, Artificial Sequence
  Synthetic primer
gttgctgtagccaaattcgttgt